(19) United States
(12) Patent Application Publication
(10) Pub. No.: US 2008/0194524 A1
(43) Pub. Date: Aug. 14, 2008

(54) Compounds For the Inhibition of Undesired Cell Proliferation and Use Thereof

(75) Inventors: Jochen Knolle, Berlin (DE); Mike Schutkowski, Ziegelroda (DE); Gerd Hummel, Berlin (DE); Thomas Tradler, Berlin (DE); Laurence Jobron, Berlin (DE); Claudia Christner, Berlin (DE); Christoph Gibson, Berlin (DE); Gunther Zischinsky, Berlin (DE)

Correspondence Address: ROTHWELL, FIGG, ERNST & MANBECK, P.C., 1425 K STREET, N.W., SUITE 800, WASHINGTON, DC, 20005, US

(73) Assignee: JERINI AG, Berlin (DE)

(21) Appl. No.: 10/583,442

(22) PCT Filed: Dec. 18, 2004
(86) PCT No.: PCT/EP2004/014460

§ 371 Date: Mar. 28, 2007

(30) Foreign Application Priority Data
Dec. 19, 2003 (EP) 03029450.8

(51) Int. Cl. A61K 31/675; A61P 35/00; A61P 29/00; A61P 37/00; A61P 31/00; A61P 9/00; A61P 25/00; C07F 9/6553; C07F 9/59; C07F 9/6558

(52) U.S. Cl. 514/85; 546/22; 544/337; 514/89

(57) ABSTRACT

The present invention is related to compounds having formula (I)

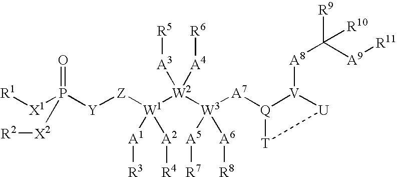

wherein the dashed line indicates a single or double bond or is absent and wherein A¹-A⁹, Q, R¹-R¹¹, T, U, V, W¹-W³, X¹, X², Y and Z are as defined in the claims and description, pharmaceutical compositions comprising the compounds and the use of the compounds for the manufacture of a medicament for the treatment of diseases involving abnormal cell proliferation, undesired cell proliferation, abnormal mitosis and/or undesired mitosis.

The present invention is related to new chemical compounds and the use of said compounds for the manufacture of medicaments.

Cell proliferation is a prerequisite for any form of life which is based on cells. Cell proliferation, i.e. increasing the cell number starting from a limited number of cells, is thus relevant for any monocellular and multicellular organism. Cell proliferation as such is a process which is highly regulated by the cell. Cell proliferation under circumstances which are not favourable to support the life of the proliferating cell has to be avoided from the biological point of view. To allow the survival of the cell complex regulation systems including sensoring mechanisms were developed in the evolution of life.

Apart from the mere increase in biomass of monocellular and multicellular organisms, multicellular organisms have to control cell proliferation in order to maintain the highly organized interaction of the cells forming the body of the multicellular organism. Any deregulation of cell proliferation represents or results in a pathological condition. Deregulated cell proliferation is the cause for a number of diseases, including the class of diseases generally referred to as cancer.

Taken the multiplicity of biological processes where cell proliferation has to be controlled, there is a need in the art to provide compounds which are suitable to control cell proliferation. The problem underlying the present invention is thus to provide new compounds which are effective in inhibiting cell proliferation, more particularly undesired cell proliferation.

In a first aspect the problem underlying the present invention is solved by a compound having formula (I)

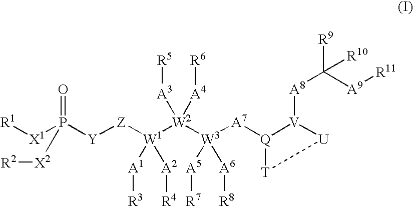

wherein the dashed line indicates a single or double bond, or is absent;
wherein R¹ and R² are each and independently selected from the group comprising —H and phospho protecting groups;
wherein X¹ and X² are each and independently selected from the group comprising —O—, —S—, —NR¹²—;
wherein Z is selected from the group comprising —O—, —S—, —NR¹³—, —(CR¹⁴R¹⁵)—;
wherein A¹, A², A³, A⁴, A⁵, A⁶, A⁷, A⁸ and A⁹ are each and independently selected from the group comprising —O—, —S—, —NR¹⁶—, —S(O)—, —S(O₂)—, —C(O)—, —C(S)—, —NR¹⁷—C(O)—, —NR¹⁸—C(S)—, —NR¹⁹—C(O)—NR²⁰—, —NR²¹C(S)—NR²²—, —NR²³—S(O)—, —NR²⁴—S(O₂)—, and —NR²⁵—C(O)—O—, or are each and independently from each other absent;
wherein Y is selected from the group comprising —O—, —CR²⁶R²⁷—;
wherein Q and V are each and independently selected from the group comprising

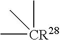

and

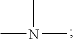

wherein W¹, W² and W³ are each and independently selected from the group comprising

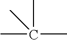

and

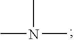

wherein R³, R⁴, R⁵, R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, R²¹, R²², R²³, R²⁴, R²⁵, R²⁶, R²⁷, R²⁸, T and U are each and independently selected from the group comprising —H, halo, alkyl, substituted alkyl, straight alkyl, substituted straight alkyl, branched alkyl, substituted branched alkyl, alkenyl, straight alkenyl, substituted straight alkenyl, branched alkenyl, substituted branched alkenyl, alkynyl, straight alkynyl, substituted straight alkynyl, branched alkynyl, substituted branched alkynyl, cycloalkyl, substituted cycloalkyl, cycloalkenyl, substituted cycloalkenyl, heterocyclyl, substituted heterocyclyl, mono-unsaturated heterocyclyl, substituted mono-unsaturated heterocyclyl, poly-unsaturated heterocyclyl, substituted poly-unsaturated heterocyclyl, aryl, substituted aryl, heteroaryl, substituted heteroaryl, arylalkyl, substituted arylalkyl, heteroarylalkyl, substituted heteroarylalkyl, heterocyclylalkyl, substituted and heterocyclylalkyl, or are each and independently from each other absent;
and the salts, hydrates, solvates and prodrugs thereof.

In an embodiment of the first aspect W¹, A¹, A², A³, A⁴, A⁵, R³, and R⁴ are absent; wherein R⁵, R⁶ and R⁷ are —H; wherein W² and W³ is

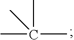

wherein preferably Z is either —S— or —O—, more preferably —S—; and wherein preferably Y is —CH₂—; wherein preferably A⁷ is either —C(O)— or —CH₂—; wherein both X¹ and X² are —O—; wherein A⁸ is —C(O)—O— or —NR²⁹—C(O)—, whereby the C-atom of the —NR²⁹—C(O)— and —C(O)—O— is covalently bound to V; and
wherein R²⁹ is —H or lower alkyl.

In an alternative embodiment of the first aspect W¹, W², A¹, A², A³, A⁴, A⁵, R³, R⁴, R⁵, and R⁶ are absent; wherein R⁷ is —H; wherein W³ is

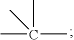

wherein preferably Z is either —S— or —O—, more preferably —S—; and wherein preferably Y is —CH₂—; wherein preferably A⁷ is either —C(O)— or —CH₂—; wherein both X¹ and X² are —O—; wherein A⁸ is —C(O)—O— or —NR²⁹—C(O)—, whereby the C-atom of the —NR²⁹—C(O)— and —C(O)—O— is covalently bound to V; and wherein R²⁹ is —H or lower alkyl.

In an embodiment of the first aspect and more preferably of the two preceding embodiments R⁸ is —H and wherein preferably A⁶ is absent.

In an embodiment of the first aspect and more preferably of the two preceding embodiments A⁶ is selected from the group comprising —NR¹⁷—C(O)—, —NR²⁴—S(O₂)—, —NR²⁵—C(O)—O—, and wherein R⁸ is selected from the group comprising optionally substituted aryl-(lower alkyl), optionally substituted heteroaryl-(lower alkyl), optionally substituted aryl and optionally substituted heteroaryl, preferably optionally substituted phenyl, optionally substituted phenyl-(lower alkyl) and more preferably 1-acetylamino-2-benzo[b]thiophen-3-yl-ethyl; dihalo-benzylsulfanylethyl, monohalo-benzylsulfanylethyl, -4-(monohalo-phenyl)-4-oxo-butyl, 4-(dihalo-phenyl)-4-oxo-butyl; benzo[1,3]dioxol-5-ylmethyl, wherein R¹⁷, R²⁴ and R²⁵ are each and independently selected from the group comprising —H and lower alkyl

In an embodiment of the first aspect and more preferably of the two preceding embodiments Q and V are

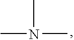

wherein T and U are alkyl, preferably lower alkyl, and wherein the dashed line is absent or a single bond.

In a preferred embodiment R²⁸ is —H or lower alkyl, wherein the dashed line is a single bond, wherein T is —CH₂— and wherein U is selected from the group comprising —(CH₂)ₙ—; wherein n is any integer from 1 to 5 and preferably 2, 3 or 4.

In a preferred embodiment R⁹ is —H and wherein R¹⁰ is selected from the group comprising substituted lower alkyl, preferably aryl-(lower-alkyl), heteroaryl-(lower-alkyl), cycloalkyl-(lower-alkyl), heterocyclyl-(lower-alkyl), and more preferably optionally substituted 2-naphthalen-2-yl-ethyl, optionally substituted naphthalen-2-ylmethyl, optionally substituted 2-phenyl-ethyl, optionally substituted 2-phenyl-methyl, optionally substituted quinolin-7-ylmethyl-, and optionally substituted 3-isoquinolin-7-ylmethyl.

In a preferred embodiment A⁹ is —NH—C(O)— or NH—C(S)—, whereby the N-atom of each of —NH—C(O)— and NH—C(S)— is covalently bound to R¹¹, and wherein R¹¹ is selected from the group comprising —H, optionally substituted alkyl, optionally substituted aryl, optionally substituted heteroaryl, optionally substituted heterocyclyl, preferably optionally substituted lower alkyl or —H, and more preferably optionally substituted tert-butyl or optionally substituted isopropyl.

In a preferred embodiment A⁹ is absent and wherein R¹¹ is selected from the group comprising optionally substituted alkyl, optionally substituted aryl, optionally substituted heteroaryl, optionally substituted heterocyclyl, preferably optionally substituted phenyl, optionally substituted thiazol-2-yl, optionally substituted pyridyl and optionally substituted [1,3,4]oxadiazol-2-yl, optionally substituted 4H-[1,2,4]triazol-3-yl.

In a preferred embodiment both R¹ and R² are —H.

In a preferred embodiment R¹ and R² are each a phospho protecting group, whereby preferably R1 and R2 are each and independently selected from the group comprising 2,2-dimethyl-propionyloxymethyl, isopropoxycarbonyloxymethyl, and 2-acetylsulfanyl-ethyl.

In a second aspect the problem underlying the present invention is solved by a compound, which is preferably a compound according to the first aspect, selected from the group comprising
{2-(2-Acetylamino-3-benzo[b]thiophen-3-yl-propionylamino)-3-[2-(1-carbamoyl-2-naphthalen-2-yl-ethylcarbamoyl)-piperidin-1-yl]-3-oxo-propoxymethyl}-phosphonic acid
{2-(2-Acetylamino-3-benzo[b]thiophen-3-yl-propionylamino)-3-[2-(1-carbamoyl-2-naphthalen-2-yl-ethylcarbamoyl)-piperidin-1-yl]-3-oxo-propylsulfanylmethyl}-phosphonic acid
{2-(2-Acetylamino-3-benzo[b]thiophen-3-yl-propionylamino)-3-[2-(1-carbamoyl-2-naphthalen-2-yl-ethylcarbamoyl)-piperidin-1-yl]-3-oxo-propylsulfanylmethyl}-phosphonic acid
[3-[2-(1-Carbamoyl-2-naphthalen-2-yl-ethylcarbamoyl)-piperidin-1-yl]-2-(9H-fluoren-9-ylmethoxycarbonylamino)-3-oxo-propoxymethyl]-phosphonic acid
{3-[2-(1-Carbamoyl-2-naphthalen-2-yl-ethylcarbamoyl)-piperidin-1-yl]-2-[3-(4-chloro-benzylsulfanyl)-propionylamino]-3-oxo-propylsulfanylmethyl}-phosphonic acid
{3-[2-(1-Carbamoyl-2-naphthalen-2-yl-ethylcarbamoyl)-piperidin-1-yl]-2-[3-(3.4-dichloro-benzylsulfanyl)-propionylamino]-3-oxo-propylsulfanylmethyl}-phosphonic acid
{3-[2-(1-Carbamoyl-2-naphthalen-2-yl-ethylcarbamoyl)-piperidin-1-yl]-2-[5-(4-chloro-phenyl)-5-oxo-pentanoylamino]-3-oxo-propylsulfanylmethyl}-phosphonic acid
[3-[2-(1-Carbamoyl-2-naphthalen-2-yl-ethylcarbamoyl)-piperidin-1-yl]-3-oxo-2-(5-phenyl-pentanoylamino)-propylsulfanylmethyl]-phosphonic acid
{2-(3-Benzo[b]thiophen-3-yl-2-{6-[5-(-2-(6-hydroxy-3-oxo-3H-xanthen-9-yl)-benzoic acid)-ureido]-hexanoylamino}-propionylamino)-3-[2-(1-carbamoyl-2-naphthalen-2-yl-ethylcarbamoyl)-piperidin-1-yl]-3-oxo-propylsulfanylmethyl}-phosphonic acid
{3-[2-(1-Carbamoyl-2-naphthalen-2-yl-ethylcarbamoyl)-piperidin-1-yl]-2-[2-(2.5-dioxo-imidazolidin-4-yl)-acetylamino]-3-oxo-propylsulfanylmethyl}-phosphonic acid
[3-[2-(1-Carbamoyl-2-naphthalen-2-yl-ethylcarbamoyl)-piperidin-1-yl]-2-(2-cyclohexyl-acetylamino)-3-oxo-propylsulfanylmethyl]-phosphonic acid
{3-[2-(1-Carbamoyl-2-naphthalen-2-yl-ethylcarbamoyl)-piperidin-1-yl]-3-oxo-2-[(2-oxo-thiazolidine-4-carbonyl)-amino]-propylsulfanylmethyl}-phosphonic acid
(3-[2-(1-Carbamoyl-2-naphthalen-2-yl-ethylcarbamoyl)-piperidin-1-yl]-3-oxo-2-{[2-oxo-3-(2-oxo-thiazolidine-4-carbonyl)-thiazolidine-4-carbonyl]-amino}-propylsulfanylmethyl)-phosphonic acid
[3-[2-(1-Carbamoyl-2-naphthalen-2-yl-ethylcarbamoyl)-piperidin-1-yl]-3-oxo-2-(3-phenoxy-benzoylamino)-propylsulfanylmethyl]-phosphonic acid
{3-[2-(1-Carbamoyl-2-naphthalen-2-yl-ethylcarbamoyl)-piperidin-1-yl]-3-oxo-2-[(1.2.3.4-tetrahydro-naphthalene-2-carbonyl)-amino]-propylsulfanylmethyl}-phosphonic acid
[3-[2-(1-Carbamoyl-2-naphthalen-2-yl-ethylcarbamoyl)-piperidin-1-yl]-3-oxo-2-(3-thiophen-2-yl-propionylamino)-propylsulfanylmethyl]-phosphonic acid
[3-[2-(1-Carbamoyl-2-naphthalen-2-yl-ethylcarbamoyl)-piperidin-1-yl]-2-(9H-fluoren-9-ylmethoxycarbonylamino)-3-oxo-propylsulfanylmethyl]-phosphonic acid
{2-{3-Benzo[b]thiophen-3-yl-2-[(piperidine-4-carbonyl)-amino]-propionylamino}-3-[2-(1-carbamoyl-2-naphthalen-2-yl-ethylcarbamoyl)-piperidin-1-yl]-3-oxo-propylsulfanylmethyl}-phosphonic acid
{2-[3-Benzo[b]thiophen-3-yl-2-(2-piperazin-1-yl-acetylamino)-propionylamino]-3-[2-(1-carbamoyl-2-naphthalen-2-yl-ethylcarbamoyl)-piperidin-1-yl]-3-oxo-propylsulfanylmethyl}-phosphonic acid
{2-Benzoylamino-3-[2-(1-carbamoyl-2-naphthalen-2-yl-ethylcarbamoyl)-piperidin-1-yl]-3-oxo-propylsulfanylmethyl}-phosphonic acid
{3-[2-(1-Carbamoyl-2-naphthalen-2-yl-ethylcarbamoyl)-piperidin-1-yl]-3-oxo-2-phenylacetylamino-propylsulfanylmethyl}-phosphonic acid
[3-[2-(1-Carbamoyl-2-naphthalen-2-yl-ethylcarbamoyl)-piperidin-1-yl]-3-oxo-2-(3-phenyl-propionylamino)-propylsulfanylmethyl]-phosphonic acid
[3-[2-(1-Carbamoyl-2-naphthalen-2-yl-ethylcarbamoyl)-piperidin-1-yl]-3-oxo-2-(4-phenyl-butyrylamino)-propylsulfanylmethyl]-phosphonic acid
{2-(2-Biphenyl-4-yl-acetylamino)-3-[2-(1-carbamoyl-2-naphthalen-2-yl-ethylcarbamoyl)-piperidin-1-yl]-3-oxo-propylsulfanylmethyl}-phosphonic acid
{2-(2-Acetylamino-3-benzo[b]thiophen-3-yl-propionylamino)-3-[2-(1-carbamoyl-2-naphthalen-2-yl-ethylcarbamoyl)-piperidin-1-yl]-propylsulfanylmethyl}-phosphonic acid
Ac-Bta-Cys(CH₂—P(O)(OH)₂)—NMeazaAla-2Nal-NH₂
Ac-Bta-Cys(CH₂—P(O)(OH)₂)—NMeazaGly-2Nal-NH₂
{2-[2-(1-Carbamoyl-2-naphthalen-2-yl-ethylcarbamoyl)-piperidin-1-yl]-2-oxo-ethylsulfanylmethyl}-phosphonic acid
{3-[2-(1-Carbamoyl-2-naphthalen-2-yl-ethylcarbamoyl)-piperidin-1-yl]-3-oxo-propylsulfanylmethyl}-phosphonic acid
{2-Acetylamino-3-[2-(1-carbamoyl-2-naphthalen-2-yl-ethylcarbamoyl)-piperidin-1-yl]-3-oxo-propylsulfanylmethyl}-phosphonic acid
{2-Benzyloxycarbonylamino-3-[2-(1-carbamoyl-2-naphthalen-2-yl-ethylcarbamoyl)-piperidin-1-yl]-3-oxo-propylsulfanylmethyl}-phosphonic acid
{3-[2-(1-Carbamoyl-2-naphthalen-2-yl-ethylcarbamoyl)-piperidin-1-yl]-3-oxo-2-phenylmethanesulfonylamino-propylsulfanylmethyl}-phosphonic acid
{3-[2-(1-Carbamoyl-2-naphthalen-2-yl-ethylcarbamoyl)-piperidin-1-yl]-3-oxo-2-[(1-phenyl-cyclopentanecarbonyl)-amino]-propylsulfanylmethyl}-phosphonic acid
{3-[2-(1-Carbamoyl-2-naphthalen-2-yl-ethylcarbamoyl)-piperidin-1-yl]-2-[2-(2-chloro-phenyl)-acetylamino]-3-oxo-propylsulfanylmethyl}-phosphonic acid
{3-[2-(1-Carbamoyl-2-naphthalen-2-yl-ethylcarbamoyl)-piperidin-1-yl]-2-[2-(4-chloro-phenyl)-acetylamino]-3-oxo-propylsulfanylmethyl}-phosphonic acid
{3-[2-(1-Carbamoyl-2-naphthalen-2-yl-ethylcarbamoyl)-piperidin-1-yl]-2-[2-(4-methoxy-phenyl)-acetylamino]-3-oxo-propylsulfanylmethyl}-phosphonic acid
{3-[2-(1-Carbamoyl-2-naphthalen-2-yl-ethylcarbamoyl)-piperidin-1-yl]-2-[4-(4-chloro-phenyl)-4-oxo-butyrylamino]-3-oxo-propylsulfanylmethyl}-phosphonic acid
{3-[2-(1-Carbamoyl-2-naphthalen-2-yl-ethylcarbamoyl)-piperidin-1-yl]-2-[4-(4-methoxy-phenyl)-butyrylamino]-3-oxo-propylsulfanylmethyl}-phosphonic acid
{3-[2-(1-Carbamoyl-2-naphthalen-2-yl-ethylcarbamoyl)-piperidin-1-yl]-3-oxo-2-[(5-oxo-pyrrolidine-2-carbonyl)-amino]-propylsulfanylmethyl}-phosphonic acid
{2-[(Benzofuran-2-carbonyl)-amino]-3-[2-(1-carbamoyl-2-naphthalen-2-yl-ethylcarbamoyl)-piperidin-1-yl]-3-oxo-propylsulfanylmethyl}-phosphonic acid
[3-[2-(1-Carbamoyl-2-naphthalen-2-yl-ethylcarbamoyl)-piperidin-1-yl]-3-oxo-2-(2-piperazin-1-yl-acetylamino)-propylsulfanylmethyl]-phosphonic acid
{2-[(3-Acetyl-2-oxo-thiazolidine-4-carbonyl)-amino]-3-[2-(1-carbamoyl-2-naphthalen-2-yl-ethylcarbamoyl)-piperidin-1-yl]-3-oxo-propylsulfanylmethyl}-phosphonic acid
{3-[2-(1-Carbamoyl-2-naphthalen-2-yl-ethylcarbamoyl)-piperidin-1-yl]-2-isobutoxycarbonylamino-3-oxo-propylsulfanylmethyl}-phosphonic acid
{2-Butoxycarbonylamino-3-[2-(1-carbamoyl-2-naphthalen-2-yl-ethylcarbamoyl)-piperidin-1-yl]-3-oxo-propylsulfanylmethyl}-phosphonic acid
{3-[2-(1-Carbamoyl-2-naphthalen-2-yl-ethylcarbamoyl)-piperidin-1-yl]-2-methoxycarbonylamino-3-oxo-propylsulfanylmethyl}-phosphonic acid
{3-[2-(1-Carbamoyl-2-naphthalen-2-yl-ethylcarbamoyl)-piperidin-1-yl]-3-oxo-2-phenoxycarbonylamino-propylsulfanylmethyl}-phosphonic acid
{3-[2-(1-Carbamoyl-2-naphthalen-2-yl-ethylcarbamoyl)-piperidin-1-yl]-3-oxo-2-phenethyloxycarbonylamino-propylsulfanylmethyl}-phosphonic acid
{2-Benzenesulfonylamino-3-[2-(1-carbamoyl-2-naphthalen-2-yl-ethylcarbamoyl)-piperidin-1-yl]-3-oxo-propylsulfanylmethyl}-phosphonic acid
[3-[2-(1-Carbamoyl-2-naphthalen-2-yl-ethylcarbamoyl)-piperidin-1-yl]-3-oxo-2-(2-phenyl-ethanesulfonylamino)-propylsulfanylmethyl]-phosphonic acid
[3-[2-(1-Carbamoyl-2-naphthalen-2-yl-ethylcarbamoyl)-piperidin-1-yl]-3-oxo-2-(3-phenyl-propane-1-sulfonylamino)-propylsulfanylmethyl]-phosphonic acid
{3-[2-(1-Carbamoyl-2-naphthalen-2-yl-ethylcarbamoyl)-piperidin-1-yl]-2-methanesulfonylamino-3-oxo-propylsulfanylmethyl}-phosphonic acid
[3-[2-(1-Carbamoyl-2-naphthalen-2-yl-ethylcarbamoyl)-piperidin-1-yl]-3-oxo-2-(2.4.6-trimethyl-benzenesulfonylamino)-propylsulfanylmethyl]-phosphonic acid
[3-[2-(1-Carbamoyl-2-naphthalen-2-yl-ethylcarbamoyl)-piperidin-1-yl]-3-oxo-2-(thiophene-2-sulfonylamino)-propylsulfanylmethyl]-phosphonic acid
[3-[2-(1-Carbamoyl-2-naphthalen-2-yl-ethylcarbamoyl)-piperidin-1-yl]-3-oxo-2-(3-piperidin-1-yl-propionylamino)-propylsulfanylmethyl]-phosphonic acid
{2-[2-(1-Carbamoyl-2-naphthalen-2-yl-ethylcarbamoyl)-piperidin-1-yl]-ethylsulfanylmethyl}-phosphonic acid
{2-(2-Benzo[1.3]dioxol-5-yl-acetylamino)-3-[2-(1-carbamoyl-2-naphthalen-2-yl-ethylcarbamoyl)-piperidin-1-yl]-3-oxo-propylsulfanylmethyl}-phosphonic acid
{3-[2-(1-Carbamoyl-2-naphthalen-2-yl-ethylcarbamoyl)-piperidin-1-yl]-2-[2-(3.5-dimethoxy-phenyl)-acetylamino]-3-oxo-propylsulfanylmethyl}-phosphonic acid
{3-[2-(1-Carbamoyl-2-naphthalen-2-yl-ethylcarbamoyl)-piperidin-1-yl]-2-[2-(2-methoxy-phenyl)-acetylamino]-3-oxo-propylsulfanylmethyl}-phosphonic acid
{2-[2-(2-Naphthalen-2-yl-ethylcarbamoyl)-piperidin-1-yl]-2-oxo-ethylsulfanylmethyl}-phosphonic acid
[2-Oxo-2-(2-phenylcarbamoyl-piperidin-1-yl)-ethylsulfanylmethyl]-phosphonic acid
{3-[2-(1-Carbamoyl-2-naphthalen-2-yl-ethylcarbamoyl)-piperidin-1-yl]-2-[2-(3-methoxy-phenyl)-acetylamino]-3-oxo-propylsulfanylmethyl}-phosphonic acid
{3-[2-(1-Carbamoyl-2-naphthalen-2-yl-ethylcarbamoyl)-piperidin-1-yl]-3-oxo-2-[2-(4-piperazin-1-yl-phenyl)-acetylamino]-propylsulfanylmethyl}-phosphonic acid
{2-[2-(1-tert-Butylcarbamoyl-2-naphthalen-2-yl-ethylcarbamoyl)-piperidin-1-yl]-2-oxo-ethylsulfanylmethyl}-phosphonic acid
{2-[2-(1-Methylcarbamoyl-2-naphthalen-2-yl-ethylcarbamoyl)-piperidin-1-yl]-2-oxo-ethylsulfanylmethyl}-phosphonic acid
{2-[2-(1-Carbamoyl-2-naphthalen-2-yl-ethylcarbamoyl)-piperidin-1-yl]-1-methyl-2-oxo-ethylsulfanylmethyl}-phosphonic acid
[2-(2-Benzylcarbamoyl-piperidin-1-yl)-2-oxo-ethylsulfanylmethyl]-phosphonic acid
[2-Oxo-2-(2-phenethylcarbamoyl-piperidin-1-yl)-ethylsulfanylmethyl]-phosphonic acid
{2-Oxo-2-[2-(3-phenyl-propylcarbamoyl)-piperidin-1-yl]-ethylsulfanylmethyl}-phosphonic acid
{2-[2-(1-Carbamoyl-2-naphthalen-2-yl-ethylcarbamoyl)-piperidin-1-yl]-1-methylcarbamoylmethyl-2-oxo-ethylsulfanylmethyl}-phosphonic acid
{2-[2-(1-Carbamoyl-2-naphthalen-2-yl-ethylcarbamoyl)-piperidin-1-yl]-1-[(4-methoxy-phenylcarbamoyl)-methyl]-2-oxo-ethylsulfanylmethyl}-phosphonic acid
{2-[2-(1-tert-Butylcarbamoyl-2-naphthalen-2-yl-ethylcarbamoyl)-piperidin-1-yl]-ethylsulfanylmethyl}-phosphonic acid
2,2-Dimethyl-propionic acid {2-[2-(1-tert-butylcarbamoyl-2-naphthalen-2-yl-ethylcarbamoyl)-piperidin-1-yl]-2-oxo-ethylsulfanylmethyl}-(2,2-dimethyl-propionyloxymethoxy)-phosphinoyloxymethyl ester
{2-[2-(2-Naphthalen-2-yl-1-phenyl-ethylcarbamoyl)-piperidin-1-yl]-2-oxo-ethylsulfanylmethyl}-phosphonic acid
(2-{2-[1-(4-Methyl-thiazol-2-yl)-2-naphthalen-2-yl-ethylcarbamoyl]-piperidin-1-yl}-2-oxo-ethylsulfanylmethyl)-phosphonic acid
{2-[2-(2-Naphthalen-2-yl-1-[1,3,4]oxadiazol-2-yl-ethylcarbamoyl)-piperidin-1-yl]-2-oxo-ethylsulfanylmethyl}-phosphonic acid
(2-{2-[2-Naphthalen-2-yl-1-(4H-[1,2,4]triazol-3-yl)-ethylcarbamoyl]-piperidin-1-yl}-2-oxo-ethylsulfanylmethyl)-phosphonic acid
{2-[2-(2-Naphthalen-2-yl-1-pyridin-2-yl-ethylcarbamoyl)-piperidin-1-yl]-2-oxo-ethylsulfanylmethyl}-phosphonic acid
   and salts, hydrates and solvates thereof as well as pro drugs thereof.

In a third aspect the problem underlying the present invention is solved by a compound according to the first or second aspect and a pharmaceutically acceptable carrier, diluent or excipient.

In an embodiment the pharmaceutical composition comprises a further pharmaceutically active compound.

In an embodiment the compound is present as a pharmaceutically acceptable salt or a pharmaceutically active solvate.

In an embodiment the pharmaceutically active compound is either alone or in combination with any of the ingredients of the composition present in a multitude of individualized dosages and/or administration forms.

In a fourth aspect the problem underlying the present invention is solved by the use of a compound according to the first or second aspect for the manufacture of a medicament.

In a fifth aspect the problem underlying the present invention is solved by the use of a compound for the manufacture of a medicament for the treatment of a disease, whereby the disease involves an abnormal cell proliferation, an undesired cell proliferation, an abnormal mitosis and/or an undesired mitosis,

whereby the compound is a compound according to the first or second aspect.

In an embodiment the compound is acting on an enzymatic activity involved in the regulation of cell division and/or cell cycle or part thereof, preferably the part of the cell cycle is mitosis.

In an embodiment the disease is selected from the group comprising neurodegenerative diseases, stroke, inflammatory diseases, immune based disorders, infectious diseases, heart diseases, cardiovascular diseases and cell proliferative diseases.

In a preferred embodiment the neurodegenerative disease is selected from the group comprising Alzheimer's disease, Huntington's disease, Parkinson's disease, peripheral neuropathy, progressive supranuclear palsy, corticobasal degeneration, frontotemporal dementia, synucleinopathies, multiple system atrophy, amyotrophic lateral atrophy, prion diseases and motor neuron diseases.

In an embodiment the infectious disease is selected from the group comprising fungal, viral, bacterial and parasite infection.

In a preferred embodiment the fungal infection is selected from the group comprising gynaecological and dermatological infection.

In an embodiment the fungal infection is caused by or involves Histoplasma, Coccidioides, Cryptococcus, Blastomyces, Paracoccidioides, Aspergillus, Sporothrix, Rhizopus, Absidia, Mucor, Hormodendrum, Phialophora Microsporum, Epidermophyton, Rhinosporidum or by a yeast, preferably Candida or Cryptococcus.

In an embodiment the infectious disease is selected from or the fungal infection causes a disorder selected from the group comprising ringworm, candidiasis, coccidioidomycosis, blastomycosis, aspergillosis, cryptococcosis, histioplasmosis, paracoccidiomycosis, zygomycosis, sporotrichiosis, mycotic keratitis, nail hair and skin disease, lobomycosis, chromoblastomycosis, mycetoma.

In a preferred embodiment the bacterial infection is selected from the group comprising infections caused by Gram-positive and by Gram-negative bacteria.

In a more preferred embodiment the bacterial infection is caused by or involves Staphylococcus, Clostridium, Streptococcus, Listeria, Salmonella, Bacillus, Escherichia, Mycobacteria, Serratia, Enterobacter, Enterococcus, Nocardia, Hemophilus, Neisseria, Proteus, Yersinia, Helicobacter or Legionella.

In an embodiment the infectious disease is selected from or the bacterial infection causes a disorder selected from the group comprising pneumonia, diarrhea, dysentery, anthrax, rheumatic fever, toxic shock syndrome, mastoiditis, meningitis, gonorrhea, typhoid fever, brucellis, Lyme disease, gastroenteritis, tuberculosis, cholera, tetanus and bubonic plague.

In a preferred embodiment the viral infection is selected from the group comprising infections caused by or involving retrovirus, HIV, Papilloma virus, Polio virus, Epstein-Barr, Herpes virus, Hepatitis virus, Papova virus, Influenza virus, Rabies, JC, encephalitis causing virus or hemorrhagic fever causing virus.

In a preferred embodiment the parasite infection is selected from the group comprising infections caused by or involving Trypanosoma, Leishmania, Trichinella, Echinococcus, Nematodes, Classes Cestoda Trematoda, Monogenea, Toxoplasma, Giardia, Balantidium, Paramecium, Plasmodium, or Entamoeba.

In an embodiment the cell proliferative disorder is selected from the group comprising neoplastic and non-neoplastic disorders.

In a preferred embodiment the neoplastic cell proliferative disorder is selected from the group comprising solid tumor, lymphoma and leukemia.

In a more preferred embodiment the solid tumor is selected from the group comprising carcinoma, sarcoma, osteoma, fibrosarcoma, and chondrosarcoma.

In an alternative embodiment the neoplastic cell proliferative disorder is selected from the group comprising breast cancer, prostate cancer, colon cancer, brain cancer, lung cancer, pancreatic cancer, gastric cancer, bladder cancer and kidney cancer.

In an alternative embodiment the non-neoplastic cell proliferative disorder is a fibrotic disorder, preferably the fibrotic disorder is fibrosis.

In an alternative embodiment the non-neoplastic cell proliferative disorder is selected from the group comprising prostatic hypertrophy, endometriosis, psoriasis, tissue repair and wound healing.

In an embodiment the immune based/inflammatory disease is an autoimmune disease or disorder.

In an embodiment the immune based/inflammatory disease is selected from the group comprising rheumatoid arthritis, glomerulonephritis, systemic lupus erythematosus associated glomerulonephritis, irritable bowel syndrome, bronchial asthma, multiple sclerosis, pemphigus, pemphigoid, scleroderma, myasthenia gravis, autoimmune haemolytic and thrombocytopenic states, Goodpasture's syndrome, pulmonary hemorrhage, vasculitis, Crohn's disease and dermatomyositis.

In an embodiment the immune based and/or inflammatory disease is an inflammatory condition.

In an embodiment the immune based and/or inflammatory disease is selected from the group comprising inflammation associated with burns, lung injury, myocardial infarction, coronary thrombosis, vascular occlusion, post-surgical vascular reocclusion, artherosclerosis, traumatic central nervous system injury, ischemic heart disease and ischemia-reperfusion injury, acute respiratory distress syndrome, systemic inflammatory response syndrome, multiple organ dysfunction syndrome, tissue graft rejection and hyperacute rejection of transplanted organs.

In an embodiment the medicament is for administration via an administration route which is selected from the group comprising oral, subcutaneous, intravenous, intranasal, transdermal, intraperitoneal, intramuscular, intrapulmonar, vaginal, rectal, and intraocular administration.

In an embodiment the medicament is for the administration to a mammal, preferably to a human being.

In an embodiment the medicament is or comprises a pharmaceutical composition according to the third aspect.

Even more preferred compounds according to the present invention are those mentioned in any of the tables herein and those further disclosed and/or characterized in the examples.

The problem underlying the present invention is also solved by the subject matter of the independent claims. Preferred embodiments may be taken from the dependent claims.

As used herein, each of the following terms, used alone or in conjunction with other terms, are preferably used in the following meaning (except where noted to the contrary):

The term “alkyl” refers to a saturated aliphatic radical containing from one to fourteen carbon atoms or a mono- or polyunsaturated aliphatic hydrocarbon radical containing from two to twelve carbon atoms, containing at least one double and triple bound, respectively. “Alkyl” refers to both branched and unbranched alkyl groups. Preferred alkyl groups are straight chain alkyl groups containing from one to eight carbon atoms. More preferred alkyl groups are straight chain alkyl groups containing from one to six carbon atoms and branched alkyl groups containing from three to six carbon atoms. It should be understood that any combination term using an “alk” or “alkyl” prefix refers to analogs according to the above definition of “alkyl”. For example, terms such as “alkoxy”, “alkylthio” refer to alkyl group linked to a second group via an oxygen or sulfur atom. “Alkanoyl” refers to an alkyl group linked to a carbonyl group (C═O). “Substituted alkyl” refers to alkyl groups as defined herein, preferably straight or branched, further bearing one or more substituents. One substituent also means mono-substituted and more substitutents mean poly-substituted. It should be understood that any combination term using a “substituted alkyl” prefix refers to analogs according to the above definition of “substituted alkyl”. For example, a term such as “substituted alkylaryl” refers to substituted alkyl group linked to an aryl group.

The term “lower alkyl” as used herein is preferably any alkyl as disclosed herein, whereby the alkyl comprises one to six, preferably one to five, and more preferably one or four C-atoms.

The term “cycloalkyl” refers to the cyclic analog of an alkyl group, as defined above, optionally unsaturated and/or substituted. Preferred cycloalkyl groups are saturated cycloalkyl groups, more particularly those containing from three to eight carbon atoms, and even more preferably three to six carbon atoms. “Substituted cycloalkyl” refers to cycloalkyl groups further bearing one or more substituents. “Mono-unsaturated cycloalkyl” refers to cycloalkyl containing one double bond or one triple bond. “Poly-unsaturated cycloalkyl” refers to cycloalkyl containing at least two double bonds or two triple bonds or a combination of at least one double bond and one triple bond.

The term “alkenyl” refers to an unsaturated hydrocarbon group containing at least one carbon-carbon double bond, including straight-chain, branched-chain, and cyclic groups. Preferred alkenyl groups have one to twelve carbons. More preferred alkenyl groups have one to six carbons. “Substituted alkenyl” refers to alkenyl groups further bearing one or more substitutents.

The term “cycloalkenyl” refers to the cyclic analog of an alkenyl group, as defined above, optionally substituted. Preferred cycloalkenyl groups are containing from four to eight carbon atoms. “Substituted cycloalkenyl” refers to cycloalkenyl groups further bearing one or more substituents. “Mono-unsaturated cycloalkenyl” refers to cycloalkenyl containing one double bond. “Poly-unsaturated cycloalkenyl” refers to cycloalkenyl containing at least two double bonds.

The term “alkynyl” refers to an unsaturated hydrocarbon group containing at least one carbon-carbon triple bond, including straight-chain, branched-chain, and cyclic groups. Preferred alkynyl groups have one to twelve carbons. More preferred alkynyl groups have one to six carbons. “Substituted alkynyl” refers to alkynyl groups further bearing one or more substitutents.

The term “aryl” refers to an aromatic group having in the range of 6 to 14 carbon atoms and “substituted aryl” refers to an aryl group further bearing one or more substituents. It should be understood that any combination term using an “ar” or “aryl” prefix refers to analogs according to the above definition of “aryl”. For example, a term such as “aryloxy” refers to an aryl group linked to a second group via an oxygen.

Each of the above defined “alkyl”, “cycloalkyl”, and “aryl” shall be understood to include their halogenated analogs, whereby the halogenated analogs may comprise one or several halogen atoms. The halogenated analogs thus comprise any halogen radical as defined in the following.

The term “halo” refers to a halogen radical selected from fluoro, chloro, bromo, iodo. Preferred halo groups are fluoro, chloro and bromo.

The term “heteroaryl” refers to a stable 5 to 8 membered, preferably 5 or 6 membered monocyclic or 8 to 11 membered bicyclic aromatic heterocycle radical. Each heterocycle consists of carbon atoms and from 1 to 4 heteroatoms selected from the group consisting of nitrogen, oxygen, and sulfur. The heterocycle may be attached by any atom of the cycle, which preferably results in the creation of a stable structure. Preferred heteroaryl radicals as used herein include, for example, furanyl, thienyl, pyrrolyl, oxazolyl, thiazolyl, imidazolyl, pyrazolyl, isoxazolyl, isothiazolyl, oxadiazolyl, triazolyl, tetrazolyl, thiadiazolyl, pyridinyl, pyridazinyl, pyrimidinyl, pyrazinyl, indolizinyl, indolyl, isoindolyl, benzofuranyl, benzothienyl, indazolyl, benzimidazolyl, benzthiazolyl, benzoxazolyl, purinyl, quinolizinyl, quinolinyl, isoquinolinyl, cinnolinyl, phthalazinyl, quinazolinyl, quinoxalinyl, naphthridinyl, pteridinyl, carbazolyl, acridinyl, phenazinyl, phenothiazinyl and phenoxazinyl. “Substituted heteroaryl” refers to heteroaryl groups further bearing one or more substituents.

The term “heterocyclyl” refers to a stable 5 to 8 membered, preferably 5 or 6 membered monocyclic or 8 to 11 membered bicyclic heterocycle radical which may be either saturated or unsaturated, and is non-aromatic. Each heterocycle consists of carbon atom(s) and from 1 to 4 heteroatoms selected from the group consisting of nitrogen, oxygen, and sulfur. The heterocycle may be attached by any atom of the cycle, which preferably results in the creation of a stable structure. Preferred heterocycle radicals as used herein include, for example, pyrrolinyl, pyrrolidinyl, pyrazolinyl, pyrazolidinyl, piperidinyl, morpholinyl, thiomorpholinyl, pyranyl, thiopyranyl, piperazinyl, indolinyl, azetidinyl, tetrahydropyranyl, tetrahydrothiopyranyl, tetrahydrofuranyl, hexahydropyrimidinyl, hexahydropyridazinyl, 1,4,5,6-tetrahydropyrimidin-2-ylamine, dihydro-oxazolyl, 1,2-thiazinanyl-1,1-dioxide, 1,2,6-thiadiazinanyl-1,1-dioxide, isothiazolidinyl-1,1-dioxide and imidazolidinyl-2,4-dione. “Mono-unsaturated heterocyclyl” refers to heterocyclyl containing one double bond or one triple bond. “Poly-unsaturated heterocyclyl” refers to heterocyclyl containing at least two double bonds or two triple bonds or a combination of at least one double bond and one triple bond. “Substituted heterocyclyl” refers to heterocyclyl groups further bearing one or more substituents.

The term “lower-alkyl”, when associated with another moiety, shall have the same meaning as given above. For example, “aryl-(lower-alkyl)” refers to lower alky, which is substituted by an aryl.

The terms “heterocyclyl”, “heteroaryl” and “aryl”, when associated with another moiety, unless otherwise specified, shall have the same meaning as given above. For example, “aroyl” refers to phenyl or naphthyl linked to a carbonyl group (C═O).

Each aryl or heteroaryl unless otherwise specified includes its partially or fully hydrogenated derivative. For example, quinolinyl may include decahydroquinolinyl and tetrahydroquinolinyl, naphthyl may include its hydrogenated derivatives such as tetrahydronaphthyl.

As used herein above and throughout this application, “nitrogen” or “N” and “sulfur” or “S” include any oxidized form of nitrogen and sulfur and the quaternized form of any basic nitrogen sulfoxide, sulfone, nitrone, N-oxide.

As used herein a wording defining the limits of a range of length such as, e.g., “from 1 to 5” means any integer from 1 to 5, i.e. 1, 2, 3, 4 and 5. In other words, any range defined by two integers explicitly mentioned is meant to comprise and disclose any integer defining said limits and any integer comprised in said range.

As used herein the term substituted shall mean that one or more H atom of the group or compound which is substituted, is replaced by a different atom, a group of atoms, a molecule or a molecule moiety. Such atom, group of atoms, molecule or molecule moiety is also referred to herein as substituent.

The substituent can be selected from the group comprising halo, trifluoromethyl, difluoromethyl, cyano, sulfonylamino, sulfamoyl, formylamino, carbamoyl, ureido, carbamoyloxy, carboxyamino, formyl, formyloxy, carboxy, sulfonyl, alky, alkenyl, alkynyl, cycloalkyl, cycloalkenyl, heterocyclyl, mono-unsaturated heterocyclyl, poly-unsaturated heterocyclyl, aryl, heteroaryl, hydroxy, alkoxy, alkenyloxy, cycloalkoxy, cycloalkenyloxy, heterocyclyloxy, aryloxy, heteroaryloxy, amino, alkylamino, alkenylamino, cycloalkylamino, cycloalkenylamino, heterocyclylamino, arylamino, heteroarylamino, mercapto, alkylsulfanyl, alkenylsulfanyl, cycloalkylsulfanyl, cycloalkenylsulfanyl, heterocyclylsulfanyl, arylsulfanyl, and heteroarylsulfanyl. Any of the substituents may be substituted itself by any of the aforementioned substituents. This applies preferably to alkyl, alkenyl, cycloalkyl, cycloalkenyl, heterocyclyl, aryl, and heteroaryl. It is also preferred that alkoxy and alkylsulfanyl are those of a lower alkyl group. It is to be acknowledged that any of the definition provided herein also applies to any substituent.

A substituent can also be any of R¹, R², R³, R⁴, R⁵, R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, R²¹, R²², R²³, R²⁴, R²⁵, R²⁶, R²⁷, R²⁸, and R²⁹. It is also within the present invention that any substituent may in turn be substituted by a substituent. A group, structure, moiety or the like which is substituted may comprise several substituents which may either be different or the same.

As used herein in connection with an embodiment of the various aspects of the present invention the term “each and independently selected from a group” or “are independently from each other selected from the group” refers to two or more atoms, groups, substituents, moieties or whatsoever and describes that the single atom, group etc. mentioned can be selected from the group. The wording used is a truncation which avoids unnecessary repetition as otherwise for each of the atoms, groups etc. the same group definition would have to be repeated.

As used herein in connection with an embodiment of the various aspects of the present invention the term “each and individually absent” refers to two or more atoms, groups, substituents, moieties or whatsoever and describes that the single atom, group etc. mentioned can be absent regardless whether any of the other atoms, groups etc. mentioned is absent. The wording used is a truncation which avoids unnecessary repetition as otherwise for each of the atoms, groups etc. the fact that it may be absent in an embodiment of the invention would have to be repeated.

In connection with the present invention some groups such as, e.g., —(CR⁴R⁵)— or —(CR³³R³⁴)— are repeated, i.e. are repeatedly present in a compound according to the present invention. Typically such repetition occurs in such a manner that, e.g., —(CR⁴R⁵)— is repeated one or several times. In case, e.g., —(CR⁴R⁵)— is repeated one time which means that there are two consecutive groups of —(CR⁴R⁵)—, these two forms of —(CR⁴R⁵)— can be either the same or they may be different in a different embodiment which means that either R⁴ or R⁵ or both of them are different between said two —(CR⁴R⁵)— groups. If there are three or more of these groups such as, e.g., —(CR⁴R⁵)—, it is possible that all of them are different or only some or different whereas others are the same in the sense defined above. Any permutation for the arrangement for such identical or different groups is within the present invention.

It is to be acknowledged and within the present invention that any radical, group, moiety or substituent as used herein can be linked or inserted in any orientation into any of the respective formulae or compounds disclosed or described herein.

As used herein in connection with an embodiment of the various aspects of the present invention the term referring to a group, substituent, moiety, spacer or the like specifying that it “can be inserted in any orientation into any of the preceding formulae” means that the group etc. can be attached to another atom, group, substitutent, moiety spacer or the like of any of the compounds according to the present invention or any of the formulae disclosed herein via any of its ends an in particular through any of the atoms arranged at the ends of said group, substituent, moiety, spacer or the like.

This applies particularly to asymmetric groups such as those having independent of any indices the following structures —NR—C(O)—, —NR—C(S)—, —NR—S(O2)- and —NR—C(O)—O—, which can thus also be inserted as —C(O)—NR—, —C(S)—NR—, —S(O2)-NR and —O—C(O)—NR—.

It is within the present invention that the features of the various embodiments of the present invention can be realized either alone or in combination with the features of any other embodiment(s) of the present invention. Thus any combination of an/the individual feature or the combination of features of an embodiment of the present invention with an/the individual feature(s) or the combination of features of any other embodiment(s), either alone or in combination with other embodiments, shall be disclosed by the present specification.

As used herein in connection with an embodiment of the various aspects of the present invention the term referring to a group, substituent, moiety, spacer or the like specifying that it “can be inserted in any orientation into any of the preceding formulae” means that the group etc. can be attached to another atom, group, substituent, moiety spacer or the like of any of the compounds according to the present invention or any of the formulae disclosed herein via any of its ends an in particular through any of the atoms arranged at the ends of said group, substituent, moiety, spacer or the like.

It is to be understood that the term group as used herein in preferred embodiments, is also to mean radical and/or diradical or any further radical having more than two free valences. It will be acknowledged by the ones skilled in the art that the various radicals or groups are linked, preferably covalently linked, to another radical, group, component or moiety of the compound. Therefore, it is appropriate to understand that such groups are regarded as radicals. It will also acknowledged that a radical an, in principle, have either one, two, three or four free valences in case of a carbon atom, for binding to or with other such radicals, groups, compounds or moieties. It is also acknowledged by the ones skilled in the art that the number of free valences thus provided defines the number of radicals with which the first radical can form a covalent bonding. For example, if Z is O which is a diradical, Z is a diradical which can bind only to other such groups, which means that in this particular example either T and W are absent with n being 0, or V, U, and R³ being absent.

The term “phospho protecting group” preferably refers to a biolabile phosphate and/or phosphonate protecting group radical as known by the person skilled in the art and, e.g., described in Schultz, Bioorg. Med. Chem. 2003, 885-898; Zemlicka, Biochim. Biophys. Acta 2002, 1587, 276-286; Tan et al., Adv. Drug. Deliv. Rev. 1999, 117-151; Shaw et al., Pharm. Res. 1997, 14, 1824-1829; Serafinowska et al., J. Med. Chem. 1997, 38, 1372-1379; Arimilli et al., Antivir. Chem. Chemother. 1997, 557-564. Preferred biolabile phosphate and/or phosphonate protecting group radicals as used herein include, for example, 2-acetylsulfanyl-ethyl, 2-pivaloylsulfanyl-ethyl, 1-methoxycarbonyl-ethyl, 1-isopropoxycarbonyl-ethyl, 2,2-Dimethyl-propionyloxymethyl, 1-methoxymethoxycarbonyl-ethyl, 2-(2-hydroxy-ethyldisulfanyl)-ethyl, isopropoxycarbonyloxymethyl, alkoxycarbonyloxymethyl, 1-alkoxycarbonyloxy-ethyl, aryl, substituted aryl and heterocyclyl.

In a further aspect the present invention is related to a pharmaceutical composition comprising a compound according to any of the aspects of the present invention and a pharmaceutically acceptable carrier, diluent or excipient.

In an embodiment the composition comprises a further pharmaceutically active compound, preferably such further pharmaceutically active compound is a chemotherapeutic agent.

In a preferred embodiment of the composition the compound is present as a pharmaceutically acceptable salt or a pharmaceutically active solvate.

In an even more preferred embodiment the pharmaceutically active compound is either alone or in combination with any of the ingredients of the composition present in a multitude of individualized dosages and/or administration forms.

In a further aspect the present invention is related to the use of the compounds according to the present invention as a medicament and for the manufacture of a medicament, respectively. It is to be understood that any of the compounds according to the present invention can be used for the treatment of or for the manufacture of a medicament for the treatment of any of the diseases disclosed herein, irrespective of the mode of action or the causative agent involved as may be specified herein. Of course, it may particularly be used for any form of such disease where the particular causative agent is involved. Causative agent as used herein also means any agent which is observed in connection with the particular disease described and such agent is not necessarily causative in the sense that is causes the observed diseases or diseased condition.

In an embodiment the medicament is for the treatment or prevention of a disease, whereby the disease may, from a mechanistical point of view, involve an undesired cell proliferation.

This use of the compounds according to the present invention is based on the fact that the compounds according to the present invention are suitable to inhibit undesired cell proliferation. Undesired cell proliferation comprises the undesired cell proliferation of procaryotic cells as well as undesired cell proliferation of eucaryotic cells. The term undesired cell proliferation also covers the phenomenon of abnormal cell proliferation, abnormal mitosis and/or undesired mitosis. Abnormal cell proliferation means any form of cell proliferation which occurs in a manner different from normal cell proliferation. Normal cell proliferation is a cell proliferation observed under normal circumstances by the majority of cells and organisms, respectively. The same basic definition applies to abnormal mitosis.

More particularly, undesired cell proliferation and undesired mitosis mean a proliferation and a mitosis, respectively, which may be either a normal or an abnormal cell proliferation, however, in any case it is not a cell proliferation or mitosis which is desired. Desired may thus be defined by an individual such as a human being and in particular a physician, and defined within certain boundaries whereby the boundaries as such may reflect the extent of proliferation and mitosis, respectively, observed under usual conditions or in the majority of cells and organisms, respectively, or may be arbitrarily fixed or defined. Cell proliferation as used herein refers preferably to the proliferation of cells forming the organism to be treated or to which a compound according to the present invention shall be administered which is also referred to herein as the first organism. Cell proliferation as used herein also means the proliferation of cells which are different from the cells forming a first organism or species but are the cells forming a second organism or second species. Typically, the second organism enters in or has a relationship with the first organism. Preferably, the first organism is a human being or an animal or plant, also referred to herein as patient, and the second organism is a parasite and pathogen, respectively, to said first organism. Mitosis as used herein, preferably means the cell division of cells being subject to said cell proliferation whereby even more preferably mitosis is the process of cell division whereby a complete set of chromosomes is distributed to the daughter cells.

Without wishing to be bound by any theory, it seems that the compounds according to the present invention act on cells and thus influence their proliferation and mitosis, respectively, by being inhibitors to some enzymatic activity. Preferably, the inhibition is reversible. This activity is shown by the compounds according to the present invention with regard to bacteria, fungi, insect and mammalian cells.

Because of this, the compounds according to the present invention may be used for the treatment of a wide variety of disorders involving cell cycle regulation, both procaryotic and eucaryotic cell cycle regulation. The term “treatment” as used herein comprises both treatment and prevention of a disease. It also comprises follow-up treatment of a disease. Follow-up treatment is realized upon a treatment of a disease using compounds preferably different from the one according to the present invention. For example, after stimulating the growth of a cell, tissue or the like by the application of a respective compound such as, e.g., erythropoietin, it might be necessary to stop an overshooting reaction of cell proliferation which may be obtained using the compounds according to the present invention.

By “reversible” herein is meant that the inhibitor binds non-covalently to the respective enzyme, and is to be distinguished from irreversible inhibition. See Walsh, Enzymatic Reaction Mechanisms, Freeman & Co., N.Y., 1979. “Reversible” in this context is a term understood by those skilled in the art. Preferably the compounds according to the present invention are competitive inhibitors, that is, they compete with substrate in binding reversibly to the enzyme, with the binding of inhibitor and substrate being mutually exclusive.

In a preferred embodiment of the compounds according to the present invention the dissociation constant for inhibition of the enzyme(s) with the inhibitor, i.e. the compound according to the present invention, generally referred to and characterized by those in the art as Kᵢ, is at most about 100 μM. By the term “binding constant” or “dissociation constant” or grammatical equivalents herein is meant the equilibrium dissociation constant for the reversible association of inhibitor with enzyme. The dissociation constants are defined and determined as described below. The determination of dissociation constants is known in the art. For example, for reversible inhibition reactions such as those of the present invention, the reaction scheme is as follows:

$$E + I {{⇌}^{{k}_{1}}}_{{k}_{2}} E * I \qquad ( Equation 1 )$$

The enzyme (E) and the inhibitor (I) combine to give an enzyme-inhibitor complex (E*I). This step is assumed to be rapid and reversible, with no chemical changes taking place; the enzyme and the inhibitor are held together by non-covalent forces. In this reaction, k₁ is the second order rate constant for the formation of the E*I reversible complex. k₂ is the first order rate constant for the dissociation of the reversible E*I complex. In this reaction, Ki=k₂/k₁.

The measurement of the equilibrium constant Kᵢ proceeds according to techniques well known in the art. For example, assays generally use synthetic chromogenic or fluorogenic substrates. The respective Kᵢ values may be estimated using the Dixon plot as described by Irwin Segel in Enzyme Kinetics: Behavior and analysis of rapid equilibrium and steady-state enzyme systems, 1975, Wiley-Interscience Publication, John Wiley & Sons, New York, or for competitive binding inhibitors from the following calculation:

1−(νᵢ/νₒ)=[I]/[I]+Kᵢ(1+([S]/Kₘ))) (Equation 2)

wherein νₒ is the rate of substrate hydrolysis in the absence of inhibitor, and νᵢ is the rate in the presence of competitive inhibitor.

The compounds according to the present invention may be easily screened for their efficacy in relation to the various uses disclosed herein

By a “labelled compound according to the present invention” herein is meant a compound according to the present invention that has at least one element, isotope or chemical compound attached to enable the detection of the compound or the compound bound to a target such as an enzyme. In general, labels as used herein, fall into three classes: a) isotopic labels, which may be radioactive or heavy isotopes; b) immune labels, which may be antibodies or antigens; and c) colored or fluorescent dyes. The labels may be incorporated into the compound at any position. Examples of useful labels include ¹⁴C, ¹³C, ¹⁵N, ³H, biotin, and fluorescent labels as are well known in the art.

As used herein, the term “disease” describes any disease, diseased condition or pathological condition. Such disease may also be defined as abnormal condition. Also, in case of a pathogen, disease means a condition where a pathogen or an unwanted organism is present or present in a concentration or compartment where it is undesired and thus subject to reduction in numbers, removal, elimination and/or destruction by using the compounds according to the present invention.

The compounds according to the present invention may be used as a medicament and for the manufacture of a medicament, respectively, whereby the medicament is preferably for the treatment of cell proliferative disorders and any of the diseases specified herein, whereby the diseases are not limited to those being cell proliferative disorders. Cell proliferated disorders as used herein, typically involve an abnormal cell proliferation, an undesired cell proliferation, an abnormal mitosis and/or an undesired mitosis.

Cell proliferative disorders contemplated for treatment using the compounds according to the present invention and for the methods disclosed herein include also disorders characterized by unwanted or undesired, inappropriate or uncontrolled cell growth. Preferably, the disease is selected from the group comprising neurodegenerative diseases, stroke, inflammatory diseases, immune based disorders, infectious diseases, heart diseases, fibrotic disorders, cardiovascular diseases and cell proliferative diseases.

Preferably, the neurodegenerative disease is selected from the group comprising Alzheimer's disease, Huntington's disease, Parkinson's disease, peripheral neuropathy, progressive supranuclear palsy, corticobasal degeneration, frontotemporal dementia, synucleinopathies, multiple system atrophy, amyotrophic lateral atrophy, prion diseases and motor neuron diseases.

The compounds according to the present invention are additionally useful in inhibiting cell cycle (mitosis) or cell division in pathogenic organisms and are, therefore, useful for treating infectious diseases.

In a preferred embodiment the infectious is selected from the group comprising fungal, viral, bacterial and parasite infection.

Fungal infections contemplated for treatment using the compounds and methods according to the present invention include systemic fungal infections, dermatophytoses and fungal infections of the genito-urinary tract. Fungal infections, preferably systemic fungal infections, include those caused by Histoplasma, Coccidioides, Cryptococcus, Blastomyces, Paracoccidioides, Aspergillus, Nocardia, Sporothrix, Rhizopus, Absidia, Mucor, Hormodendrum, Phialophora, Rhinosporidium, and the like. Dermatophyte infections include those caused by Microsporum, Trichophyton, Epidermophyton, Candida, Pityrosporum, and the like. Fungal disorders of the genito-urinary tract include infections caused by Candida, Cryptococcus, Aspergillus, Zygomycodoides, and the like. Infection by such organisms causes a wide variety of disorders such as ringworm, thrush or candidiasis, San Joaquin fever or Valley fever or coccidiodomycosis, Gilchrist's disease or blastomycosis, aspergillosis, cryptococcosis, histioplasmosis, paracoccidiomycosis, zygomycosis, mycotic keratitis, nail hair and skin disease, Lobo's disease, lobomycosis, chromoblastomycosis, mycetoma, and the like. These infections can be particularly serious, and even fatal, in patients with a depressed immune system such as organ transplant recipients and persons with acquired immunodeficiency syndrome (AIDS). Insofar a patient group which can be treated using the inhibitors according to the present invention are persons with AIDS, particularly those suffering from any of the infectious diseases described herein.

In a further embodiment the bacterial infection is selected from the group comprising infections caused by both Gram-positive and Gram-negative bacteria, including infections caused by Staphylococcus, Clostridium, Streptococcus, Enterococcus, Diplococcus, Hemophilus, Neisseria, Erysipelothricosis, Listeria, Bacillus, Salmonella, Shigella, Escherichia, Klebsiella, Enterobacter, Serratia, Proteus, Morganella, Providencia, Yersinia, Camphylobacter, Mycobacteria, Helicobacter, Legionalla, Nocardia and the like.

In a preferred embodiment the bacterial infection causes a wide variety of diseases. Said disorders are selected, among others, from the group comprising pneumonia, diarrhea, dysentery, anthrax, rheumatic fever, toxic shock syndrome, mastoiditis, meningitis, gonorrhea, typhoid fever, brucellis, Lyme disease, gastroenteritis, tuberculosis, cholera, tetanus and bubonic plague.

In another embodiment the disease is a viral infection, more particularly a viral infection caused by a virus selected from the group comprising retrovirus, HIV, Papilloma virus, Epstein-Barr, Herpes virus, Hepatitis virus, Papova virus, Influenza virus, Rabies, JC, encephalitis causing virus, hemorrhagic fever causing virus such as Ebola Virus and Marburg Virus.

In a further embodiment the parasite infection is selected from the group comprising infections caused by Trypanosoma, Leishmania, Trichinella, Echinococcus, Nematodes, Classes Cestoda, Trematoda, Monogenea, Toxoplasma, Giardia, Balantidium, Paramecium, Plasmodium or Entamoeba.

The disease may further be a cell proliferative disorder which preferably is selected from the group characterized by unwanted, inappropriate or uncontrolled cell growth. Particular examples include cancer, fibrotic disorders, non-neoplastic growths. The neoplastic cell proliferative disorder is preferably selected from the group comprising solid tumors, and hematopoeitic cancers such as lymphoma and leukemia.

More preferably, the solid tumor is selected from the group comprising carcinoma, sarcoma, osteoma, fibrosarcoma, and chondrosarcoma.

More preferably, the cell proliferative disorder is selected from the group comprising breast cancer, prostate cancer, colon cancer, brain cancer, lung cancer, pancreatic cancer, gastric cancer, bladder cancer, kidney cancer and head and neck cancer. Preferably, the lung cancer is non-small lung cancer and small lung cancer.

In case the disease is a non-neoplastic cell proliferative disorder, it is preferably selected from the group comprising fibrotic disorder. Preferably, the fibrotic disorder is fibrosis.

The disease may also be a non-neoplastic cell proliferative disorder which is selected from the group comprising prostatic hypertrophy, preferably benign prostatic hypertrophy, endometriosis, psoriasis, tissue repair and wound healing.

Fibrotic disorders which may be treated using the compounds according to the present invention are generally characterized by inappropriate overproliferation of non-cancerous fibroblasts. Examples thereof include fibromyalgia, fibrosis, more particularly cystic, hepatic, idopathic pulmonary, and pericardial fibrosis and the like, cardiac fibromas, fibromuscular hyperplasia, restenosis, atherosclerosis, fibromyositis, and the like.

In another embodiment the immune based and/or inflammatory disease is an autoimmune disease or autoimmune disorder. In a further embodiment, the immune based and/or inflammatory disease is selected from the group comprising rheumatoid arthritis, glomerulonephritis, systemic lupus erythematosus associated glomerulonephritis, irritable bowel syndrome, bronchial asthma, multiple sclerosis, pemphigus, pemphigoid, scleroderma, myasthenia gravis, autoimmune haemolytic and thrombocytopenic states, Goodpasture's syndrome, pulmonary hemorrhage, vasculitis, Crohn's disease, and dermatomyositis.

In a further preferred embodiment the immune based and/or inflammatory disease is an inflammatory condition.

In a still further embodiment the immune based and/or inflammatory disease is selected from the group comprising inflammation associated with burns, lung injury, myocardial infarction, coronary thrombosis, vascular occlusion, post-surgical vascular reocclusion, artherosclerosis, traumatic central nervous system injury, ischemic heart disease and ischemia-reperfusion injury, acute respiratory distress syndrome, systemic inflammatory response syndrome, multiple organ dysfunction syndrome, tissue graft rejection and hyperacute rejection of transplanted organs.

It is also within the present invention that the compounds according to the present invention may be used for the treatment of a patient suffering from a disease or diseased condition as defined above. Such treatment comprises the administration of one or several of the compounds according to the present invention or a medicament or pharmaceutical composition described herein.

Toxicity and therapeutic efficacy of a compound can be determined by standard pharmaceutical procedures in cell culture or experimental animals. Cell culture assays and animal studies can be used to determine the LD₅₀ (the dose lethal to 50% of a population) and the ED₅₀ (the dose therapeutically effective in 50% of a population). The dose ratio between toxic and therapeutic effects is the therapeutic index, which can be expressed as the ratio LD₅₀/ED₅₀. Compounds which exhibit large therapeutic indices are preferred. The data obtained from these cell culture assays and animal studies can be used in formulating a range of dosages suitable for use in humans. The dosage may vary within this range depending upon a variety of factors, e.g., the dosage form employed, the route of administration utilized, the condition of the subject, and the like

For any compound according to the present invention, the therapeutically effective dose can be estimated initially from cell culture assays by determining an IC₅₀ (i.e., the concentration of the test substance which achieves a half-maximal inhibition of cell proliferation). A dose can then be formulated in animal models to achieve a circulating plasma concentration range that includes the IC₅₀ as determined in cell culture. Such information can be used to more accurately determine useful doses in humans. Levels in plasma may be measured, for example by HPLC or LC/MS.

It should be noted that the attending physician would know how to and when to terminate, interrupt, or adjust administration due to toxicity, to organ dysfunction, and the like. Conversely, the attending physician would also know to adjust treatment to higher levels if the clinical response were not adequate (precluding toxicity). The magnitude of an administered dose in the management of the disorder of interest will vary with the severity of the condition to be treated, with the route of administration, and the like. The severity of the condition may, for example, be evaluated, in part, by standard prognostic evaluation methods. Further, the dose and perhaps dose frequency will also vary according to the age, body weight, and response of the individual patient. Typically, the dose will be between about 1-10 mg/kg of body weight. About 1 mg to about 50 mg will preferably be administered to a child, and between 25 mg and about 1000 mg will preferably be administered to an adult.

A program comparable to that discussed above may be used in veterinary medicine. The exact dose will depend on the disorder to be treated and will be ascertainable by one skilled in the art using known techniques.

Depending on the specific conditions to be treated, such compounds may be formulated and administrated systemically or locally. Techniques for formulation and administration may be found in “Remington's Pharmaceutical Sciences”, 1990, 18^{th} ed., Mack Publishing Co., Easton, Pa. The administration of a compound according to the present invention can be done in a variety of ways, including, but not limited to, orally, subcutaneously, intravenously, intranasally, transdermally, intraperitoneally, intramuscularly, intrapulmonary, vaginally, rectally, or intraocularly, just to name a few. In some instances, for example, in the treatment of wounds and inflammation, the compound according to the present invention may be directly applied as a solution or spray.

In a further aspect the present invention is related to a medicament or a pharmaceutical composition comprising at least one active compound and at least one pharmaceutically acceptable carrier, excipient or diluent. As used herein, the active compound is a compound according to the present invention, a pharmaceutically salt or base thereof or a prodrug thereof, if not indicated to the contrary.

For injection, compounds of the invention may be formulated in aqueous solution, preferably in physiologically compatible buffers such as Hank's solution, Ringer's solution, or physiologically saline buffer. For transmucosal administration, penetrants appropriate to the barrier to be permeated are used in the formulation. Such penetrants are generally known in the art.

The use of pharmaceutical acceptable carriers to formulate the compounds according to the present invention into dosages or pharmaceutical compositions suitable for systemic administration is within the scope of the present invention. With proper choice of carrier and suitable manufacturing practice, the compositions of the present invention, in particular those formulated as solutions, may be administered parenterally, such as by intravenous injection. The compounds can be readily formulated using pharmaceutically acceptable carriers well known in the art into dosages suitable for oral administration. Such carriers enable the compounds according to the present invention to be formulated as tablets, pills, capsules, dragees, liquids, gels, syrups, slurries, suspensions and the like, for oral ingestion by a subject to be treated.

Compounds according to the present invention or medicaments comprising them, intended to be administered intracellularly may be administered using techniques well known to those of ordinary skill in the art. For example, such agents may be encapsulated into liposomes, then administered as described above. Liposomes are spherical lipid bilayers with aqueous interiors. All molecules present in an aqueous solution at the time of liposome formation are incorporated into the aqueous interior. The liposomal contents are both protected from the external microenvironment and, because liposomes fuse with cell membranes, are efficiently delivered into the cell cytoplasm. Delivery systems involving liposomes are disclosed in International Patent Publication No. WO 91/19501, as well as U.S. Pat. No. 4,880,635 to Janoff et al. The publications and patents provide useful descriptions of techniques for liposome drug delivery and are incorporated by reference herein in their entirety.

Pharmaceutical compositions comprising a compound according to the present invention for parenteral administration include aqueous solutions of the active compound(s) in water-soluble form. Additionally, suspensions of the active compounds may be prepared as appropriate oily injection suspensions. Suitable lipophilic solvents or vehicles include fatty oils such as sesame oil or castor oil, or synthetic fatty acid esters, such as ethyl oleate or triglycerides, or liposomes. Aqueous injections suspensions may contain compounds which increase the viscosity of the suspension, such as sodium carboxymethyl cellulose, sorbitol, dextran, or the like. Optionally, the suspension may also contain suitable stabilizers or agents which increase the solubility of the compounds to allow for the preparation of highly concentrated solutions.

Pharmaceutical compositions comprising a compound according to the present invention for oral use can be obtained by combining the active compound(s) with solid excipient, optionally grinding the resulting mixture, and processing the mixture of granules, after adding suitable auxiliaries, if desired, to obtain tablets or dragee cores.

Suitable excipients are, in particular, fillers such as sugars, including lactose, sucrose, mannitol, sorbitol, and the like; cellulose preparations, such as, for example, maize starch wheat starch, rice starch, potato starch, gelatin, gum tragacanth, methyl cellulose, hydroxypropylmethyl cellulose, sodium carboxymethyl cellulose, polyvinylpyrrolidone (PVP) and the like, as well as mixtures of any two or more thereof. If desired, disintegrating agents may be added, such as cross-linked polyvinyl pyrrolidone, agar, alginic acid or a salt thereof such as sodium alginate, and the like.

Dragee cores as a pharmaceutical composition comprising a compound according to the present invention are provided with suitable coatings. For this purpose, concentrated sugar solutions may be used, which may optionally contain gum arabic, talc, polyvinyl pyrrolidone, carbopol gel, polyethylene glycol, titanium dioxide, lacquer solutions, suitable organic solvents or solvent mixtures, and the like. Dyestuffs or pigments may be added to the tablets or dragee coatings for identification or to characterize different combinations of active compound doses.

Pharmaceutical preparations comprising a compound according to the present invention which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added.

A “patient” for the purposes of the present invention, i.e. to whom a compound according to the present invention or a pharmaceutical composition according to the present invention is administered, includes both humans and other animals and organisms. Thus the compounds, pharmaceutical compositions and methods are applicable to or in connection with both human therapy and veterinary applications including diagnostic(s), diagnostic procedures and methods as well as staging procedures and methods. For example, the veterinary applications include, but are not limited to, canine, bovine, feline, porcine, caprine, equine, and ovine animals, as well as other domesticated animals including reptiles, such as iguanas, turtles and snakes, birds such as finches and members of the parrot family, lagomorphs such as rabbits, rodents such as rats, mice, guinea pigs and hamsters, amphibians, fish, and arthropods. Valuable non-domesticated animals, such as zoo animals, may also be treated. In the preferred embodiment the patient is a mammal, and in the most preferred embodiment the patient is human.

The pharmaceutical composition according to the present invention comprises at least one compound according to the present invention in a form suitable for administration to a patient. Preferably, a compound according to the present application is in a water soluble form, such as being present as a pharmaceutically acceptable salt, which is meant to include both acid and base addition salts which are also generally referred to herein as pharmaceutically acceptable salts. “Acid addition salt”, and more particularly “pharmaceutically acceptable acid addition salts” refers to those salts that retain the biological effectiveness of the free bases and that are not biologically or otherwise undesirable, formed with inorganic acids such as hydrochloric acid, hydrobromic acid, sulfuric acid, nitric acid, phosphoric acid and the like, and organic acids such as acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid and the like. “Base addition salts” and more particularly “pharmaceutically acceptable base addition salts” include those derived from inorganic bases such as sodium, potassium, lithium, ammonium, calcium, magnesium, iron, zinc, copper, manganese, aluminum salts and the like. Particularly preferred are the ammonium, potassium, sodium, calcium, and magnesium salts. Salts derived from pharmaceutically acceptable organic non-toxic bases include salts of primary, secondary, and tertiary amines, substituted amines including naturally occurring substituted amines, cyclic amines and basic ion exchange resins, such as isopropylamine, trimethylamine, diethylamine, triethylamine, tripropylamine, and ethanolamine. The pharmaceutical compositions according to the present invention may also include one or more of the following: carrier proteins such as serum albumin; buffers; fillers such as microcrystalline cellulose, lactose, corn and other starches; binding agents; sweeteners and other flavoring agents; coloring agents; and polyethylene glycol. Additives are well known in the art, and are used in a variety of formulations.

The compounds according to the present invention are, in a further embodiment, administered to a subject either alone or in a pharmaceutical composition where the compound(s) is mixed with suitable carriers or excipient(s). In treating a subject, a therapeutically effective dose of compound (i.e. active ingredient) is administered. A therapeutically effective dose refers to that amount of the active ingredient that produces amelioration of symptoms or a prolongation of survival of a subject which can be determined by the one skilled in the art doing routine testing.

On the other hand, the compounds according to the present invention may as such or contained in a pharmaceutical composition according to the present invention be used in drug potential applications.

For example, therapeutic agents such as antibiotics or antitumor drugs can be inactivated through the catalytic action of endogenous enzymes, thus rendering the administered drug less effective or inactive. Accordingly, the compound(s) according to the present invention may be administered to a patient in conjunction with a therapeutic agent in order to potentiate or increase the activity of the drug. This co-administration may be by simultaneous administration, such as a mixture of the compound(s) according to the present invention and the drug, or by separate simultaneous or sequential administration.

According to the present invention the compounds disclosed herein, referred to as compounds according to the present invention, may be used as a medicament or for the manufacture of medicament or in a method of treatment of a patient in need thereof. Insofar any of these compounds constitute a pharmaceutical compound. The use of this kind of compound also comprises the use of pharmaceutically acceptable derivatives of such compounds.

In addition, the compounds according to the present invention may be transformed upon application to an organism such as a patient, into the pharmaceutically active compound. Insofar the compounds according to the present invention may be prodrugs which, however, are nevertheless used for the manufacture of the medicaments as disclosed herein given the fact that at least in the organism they are changed in a form which allows the desired

It is to be understood that any of the pharmaceutical compositions according to the present invention may be used for any of the diseases described herein.

The pharmaceutical compositions according to the present invention may be manufactured in a manner that is known as such, e.g., by means of conventional mixing, dissolving, granulating, dragee-mixing, levigating, emulsifying, encapsulating, entrapping, lyophilizing, processes, or the like.

In a further aspect of the present invention the compounds of the present invention may be used as insecticides as they may prevent cell cycle mitosis in insect cells and thus can be used to control the growth and proliferation of a variety of insect pests. This aspect of the present invention has important applications in agriculture, such as in the field, in the storage of agricultural products and the like. Additionally, the compounds according to the present invention are useful for controlling insect populations, preferably in places inhabited by men, such as homes, offices and the like.

Any of the compounds according to the present invention containing one or more asymmetric carbon atoms may occur as racemates and racemic mixtures, single enantiomers, diastereomeric mixtures and individual diastereomers. All such isomeric forms of these compounds are expressly included in the present invention. Each stereogenic carbon may be in the R or S configuration, or a combination of configurations.

It shall be understood by the one of ordinary skill in the art that all compounds of the invention are preferably those which are chemically stable. This applies to any of the various uses of the compounds according to the present invention disclosed herein.

In determining the suitability of any of the compounds according to the present applications for the various uses, besides the particular use-specific profile to be met by such a compound, also it has to be checked whether it is stable to proteolytic degradation. The resistance of the compound used as a pharmaceutical may be tested against a variety of non-commercially available proteases in vitro to determine its proteolytic stability. Promising candidates may then be routinely screened in animal models, for example using labelled inhibitors, to determine the in vivo stability and efficacy. In any of the aforementioned uses the compound may be present in a crude or purified form. Methods for purifying the compounds according to the present invention are known to the one skilled in the art.

Particularly preferred compounds according to the invention are set forth in table 1:

TABLE 1

| | | | | Mol | MS data |
|---|---|---|---|---|---|
| | Structure | Chemical name | Formula | Weight | [M + H⁺] |
| 1 | 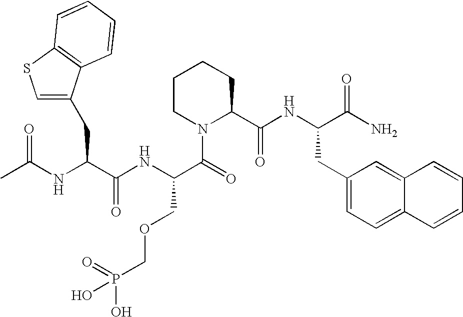 | {2-(2-Acetylamino-3-benzo[b]thiophen-3-yl-propionylamino)-3-[2-(1-carbamoyl-2-naphthalen-2-yl-ethylcarbamoyl)-piperidin-1-yl]-3-oxo-propoxymethyl}-phosphonicacid | C36H42N5O9PS | 751.79 | 752.3 |
| 2 | 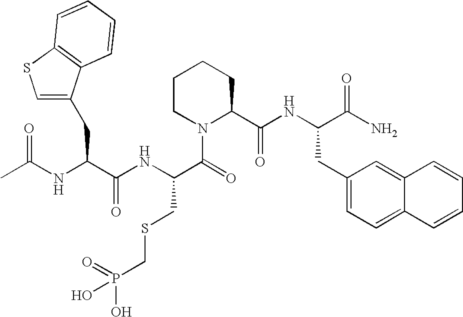 | {2-(2-Acetylamino-3-benzo[b]thiophen-3-yl-propionylamino)-3-[2-(1-carbamoyl-2-naphthalen-2-yl-ethylcarbamoyl)-piperidin-1-yl]-3-oxo-propylsulfanylmethyl}-phosphonic acid | C36H42N5O8PS2 | 767.85 | 768.3 |
| 3 | 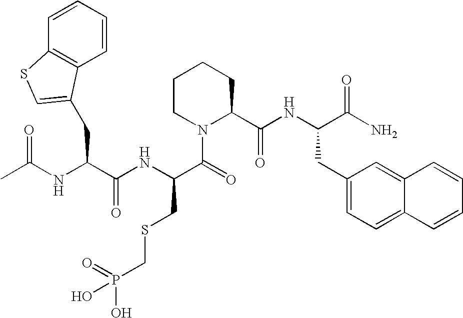 | {2-(2-Acetylamino-3-benzo[b]thiophen-3-yl-propionylamino)-3-[2-(1-carbamoyl-2-naphthalen-2-yl-ethylcarbamoyl)-piperidin-1-yl]-3-oxo-propylsulfanylmethyl}-phosphonic acid | C36H42N5O8PS2 | 767.85 | 768.4 |
| 4 | 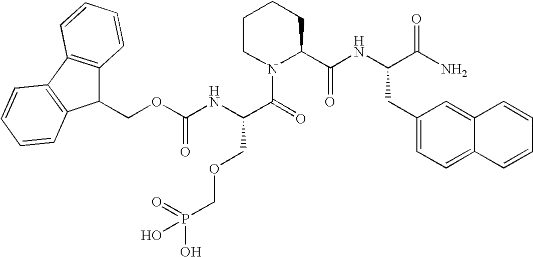 | [3-[2-(1-Carbamoyl-2-naphthalen-2-yl-ethylcarbamoyl)-piperidin-1-yl]-2-(9H-fluoren-9-ylmethoxycarbonylamino)-3-oxo-propoxymethyl]-phosphonic acid | C38H41N4O9P | 728.73 | 729.3 |
| 5 | 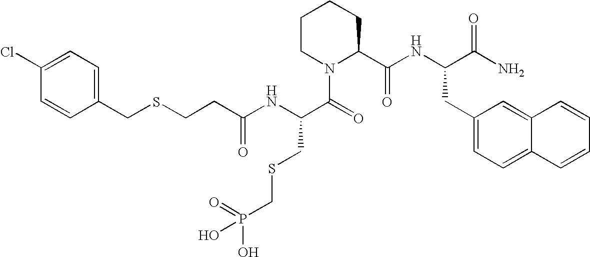 | {3-[2-(1-Carbamoyl-2-naphthalen-2-yl-ethylcarbamoyl)-piperidin-1-yl]-2-[3-(4-chloro-benzylsulfanyl)-propionylamino]-3-oxo-propylsulfanylmethyl}-phosphonicacid | C33H40ClN4O7PS2 | 735.25 | 735.3 |
| 6 | 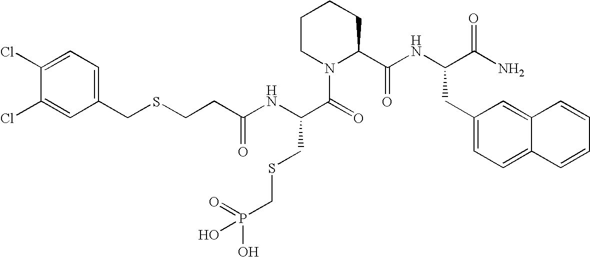 | {3-[2-(1-Carbamoyl-2-naphthalen-2-yl-ethylcarbamoyl)-piperidin-1-yl]-2-[3-(3.4-dichloro-benzylsulfanyl)-propionylamino]-3-oxo-propylsulfanylmethyl}-phosphonic acid | C33H39Cl2N4O7PS2 | 769.70 | 769.2 |
| 7 | 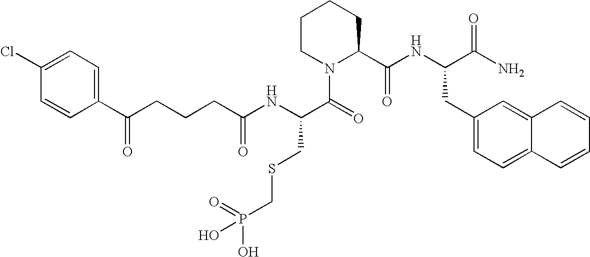 | {3-[2-(1-Carbamoyl-2-naphthalen-2-yl-ethylcarbamoyl)-piperidin-1-yl]-2-[5-(4-chloro-phenyl)-5-oxo-pentanoylamino]-3-oxo-propylsulfanylmethyl}-phosphonicacid | C34H40ClN4OSPS | 731.20 | 731.2 |
| 8 | 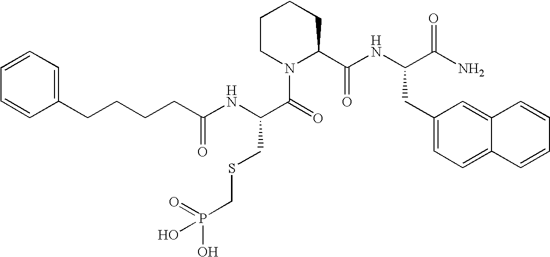 | [3-[2-(1-Carbamoyl-2-naphthalen-2-yl-ethylcarbamoyl)-piperidin-1-yl]-3-oxo-2-(5-phenyl-pentanoylamino)-propylsulfanylmethyl]-phosphonicacid | C34H43N4O7PS | 682.77 | 683.3 |
| 9 | 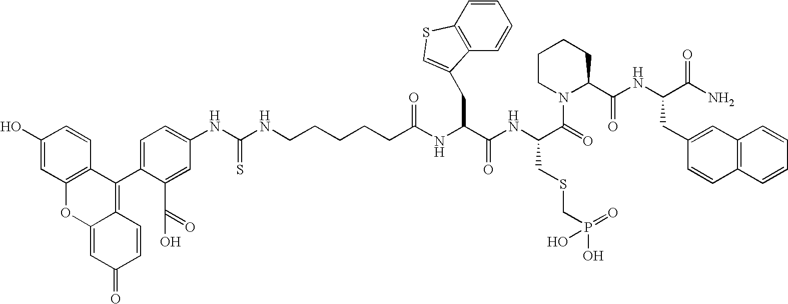 | {2-(3-Benzo[b]thiophen-3-yl-2-{6-[5-(-2-(6-hydroxy-3-oxo-3H-xanthen-9-yl)-benzoic acid)-ureido]-hexanoylamino}-propionylamino)-3-[2-(1-carbamoyl-2-naphthalen-2-yl-ethylcarbamoyl)-piperidin-1-yl]-3-oxo-propylsulfanylmethyl}-phosphonic acid | C61H62N7O13PS3 | 1228.35 | 1228.4 |
| 10 | 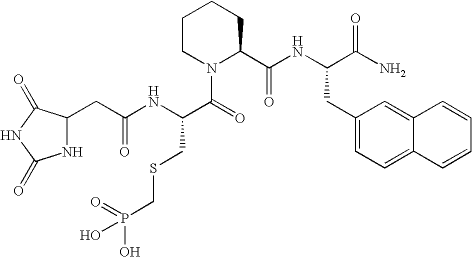 | {3-[2-(1-Carbamoyl-2-naphthalen-2-yl-ethylcarbamoyl)-piperidin-1-yl]-2-[2-(2.5-dioxo-imidazolidin-4-yl)-acetylamino]-3-oxo-propylsulfanylmethyl}-phosphonicacid | C28H35N6O9PS | 662.65 | 663.3 |
| 11 | 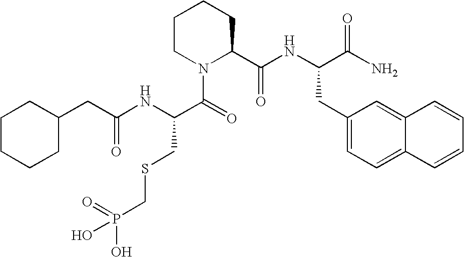 | [3-[2-(1-Carbamoyl-2-naphthalen-2-yl-ethylcarbamoyl)-piperidin-1-yl]-2-(2-cyclohexyl-acetylamino)-3-oxo-propylsulfanylmethyl]-phosphonic acid | C31H43N4O7PS | 646.73 | 647.5 |
| 12 | 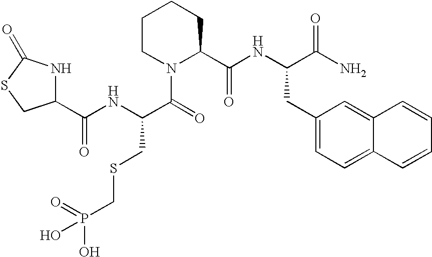 | {3-[2-(1-Carbamoyl-2-naphthalen-2-yl-ethylcarbamoyl)-piperidin-1-yl]-3-oxo-2-[(2-oxo-thiazolidine-4-carbonyl)-amino]-propylsulfanylmethyl}-phosphonicacid | C27H34N5O8PS2 | 651.69 | 652.2 |
| 13 | 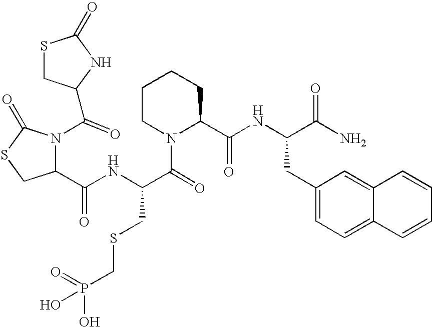 | (3-[2-(1-Carbamoyl-2-naphthalen-2-yl-ethylcarbamoyl)-piperidin-1-yl]-3-oxo-2-{[2-oxo-3-(2-oxo-thiazolidine-4-carbonyl)-thiazolidine-4-carbonyl]-amino}-propylsulfanylmethyl)-phosphonicacid | C31H37N6O10PS3 | 730.83 | 781.2 |
| 14 | 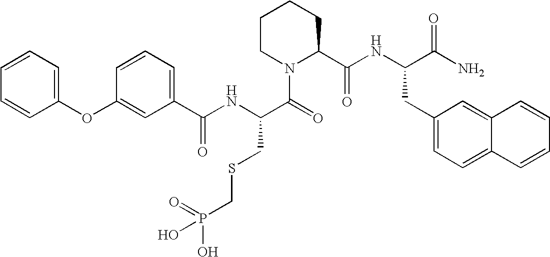 | [3-[2-(1-Carbamoyl-2-naphthalen-2-yl-ethylcarbamoyl)-piperidin-1-yl]-3-oxo-2-(3-phenoxy-benzoylamino)-propylsulfanylmethyl]-phosphonicacid | C36H39N4O8PS | 718.76 | 719.5 |
| 15 | 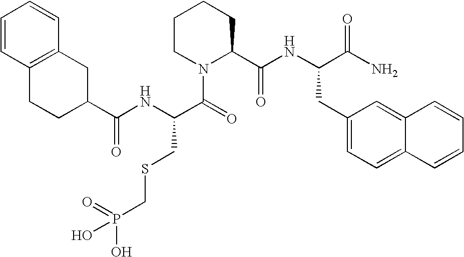 | {3-[2-(1-Carbamoyl-2-naphthalen-2-yl-ethylcarbamoyl)-piperidin-1-yl]-3-oxo-2-[(1.2.3.4-tetrahydro-naphthalene-2-carbonyl)-amino]-propylsulfanylmethyl}-phosphonicacid | C34H41N4O7PS | 680.75 | 681.3 |
| 16 | 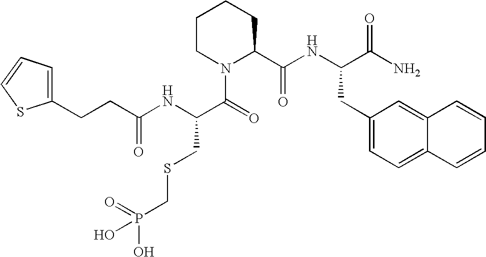 | [3-[2-(1-Carbamoyl-2-naphthalen-2-yl-ethylcarbamoyl)-piperidin-1-yl]-3-oxo-2-(3-thiophen-2-yl-propionylamino)-propylsulfanylmethyl]-phosphonicacid | C30H37N4O7PS2 | 660.74 | 661.4 |
| 17 | 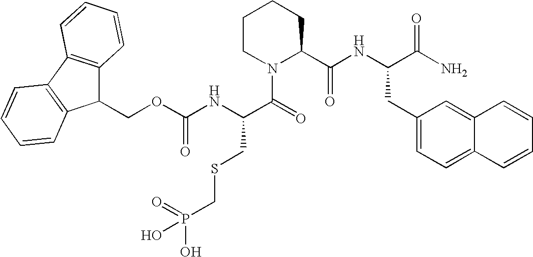 | [3-[2-(1-Carbamoyl-2-naphthalen-2-yl-ethylcarbamoyl)-piperidin-1-ylmethoxycarbonylamino)-3-oxo-propylsulfanylmethyl]-phosphonicacid | C38H41N4O8PS | 744.79 | 745.4 |
| 18 | 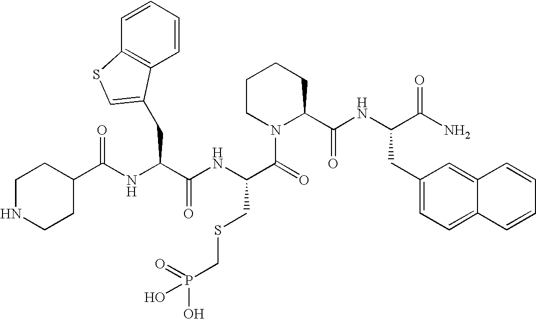 | {2-{3-Benzo[b]thiophen-3-yl-2-[(piperidine-4-carbonyl)-amino]-propionylamino}-3-[2-(1-carbamoyl-2-naphthalen-2-yl-ethylcarbamoyl)-piperidin-1-yl]-3-oxo-propylsulfanylmethyl}-phosphonic acid | C40H49N6O8PS2 | 836.96 | 837.5 |
| 19 | 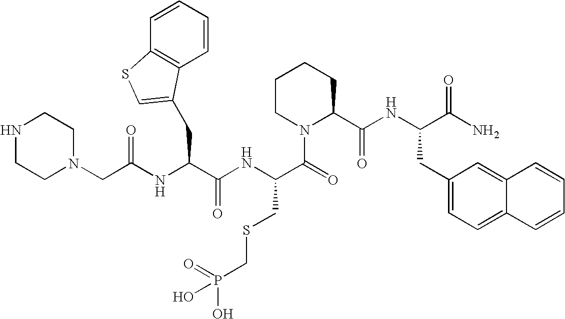 | {2-[3-Benzo[b]thiophen-3-yl-2-(2-piperazin-1-yl-acetylamino)-propionylamino]-3-[2-(1-carbamoyl-2-naphthalen-2-yl-ethylcarbamoyl)-piperidin-1-yl]-3-oxo-propylsulfanylmethyl}-phosphonic acid | C40H50N7O8PS2 | 851.97 | 852.4 |
| 20 | 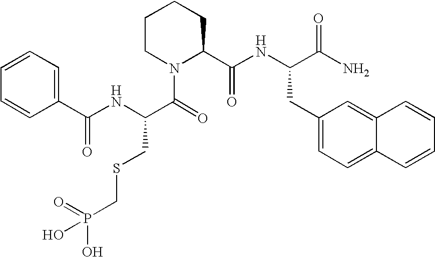 | {2-Benzoylamino-3-[2-(1-carbamoyl-2-naphthalen-2-yl-ethylcarbamoyl)-piperidin-1-yl]-3-oxo-propylsulfanylmethyl}-phosphonic acid | C30H35N4O7PS | 626.66 | 627.1 |
| 21 | 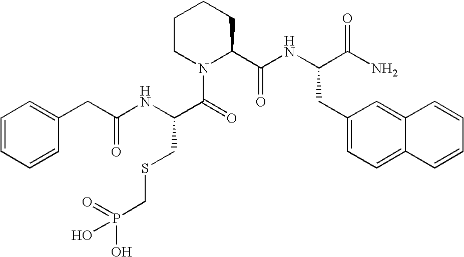 | {3-[2-(1-Carbamoyl-2-naphthalen-2-yl-ethylcarbamoyl)-piperidin-1-yl]-3-oxo-2-phenylacetylamino-propylsulfanylmethyl}-phosphonicacid | C31H37N4O7PS | 640.69 | 641.5 |
| 22 | 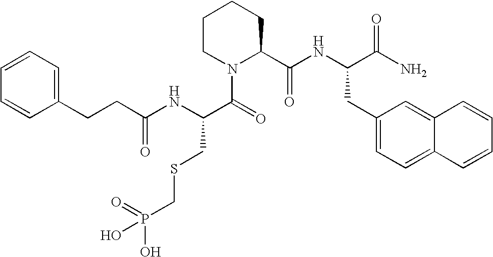 | [3-[2-(1-Carbamoyl-2-naphthalen-2-yl-ethylcarbamoyl)-piperidin-1-yl]-3-oxo-2-(3-phenyl-propionylamino)-propylsulfanylmethyl]-phosphonicacid | C32H39N4O7PS | 654.71 | 655.5 |
| 23 | 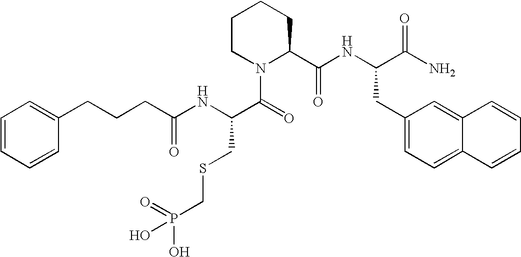 | [3-[2-(1-Carbamoyl-2-naphthalen-2-yl-ethylcarbamoyl)-piperidin-1-yl]-3-oxo-2-(4-phenyl-butyrylamino)-propylsulfanylmethyl]-phosphonicacid | C33H41N4O7PS | 668.74 | 669.6 |
| 24 | 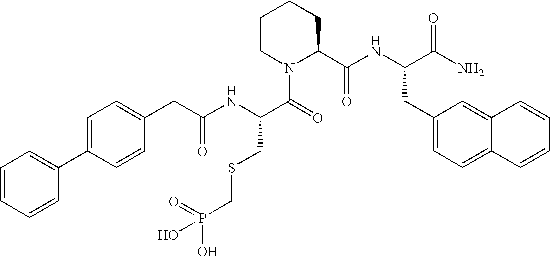 | {2-(2-Biphenyl-4-yl-acetylamino)-3-[2-(1-carbamoyl-2-naphthalen-2-yl-ethylcarbamoyl)-piperidin-1-yl]-3-oxo-propylsulfanylmethyl}-phosphonic acid | C37H41N4O7PS | 716.78 | 717.7 |
| 25 | 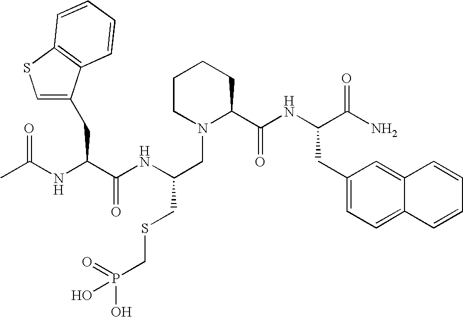 | {2-(2-Acetylamino-3-benzo[b]thiophen-3-yl-propionylamino)-3-[2-(1-carbamoyl-2-naphthalen-2-yl-ethylcarbamoyl)-piperidin-1-yl]-propylsulfanylmethyl}-phosphonicacid | C36H44N5O7PS2 | 753.87 | 754.6 |
| 26 | 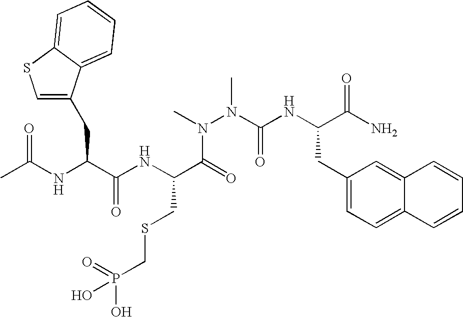 | Ac-Bta-Cys(CH₂—P(O)(OH)₂)-NMeazaAla-2Nal-NH₂ | C33H39N6O8PS2 | 742.80 | 743.4 |
| 27 | 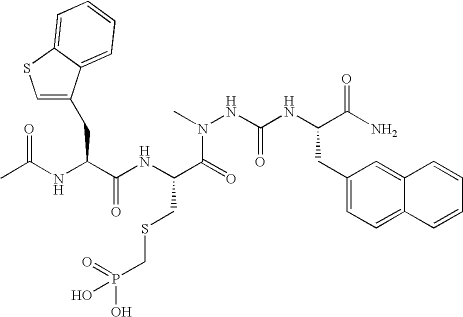 | Ac-Bta-Cys(CH₂—P(O)(OH)₂)-NMeazaGly-2Nal-NH₂ | C32H37N6O8PS2 | 728.78 | 729.5 |
| 28 | 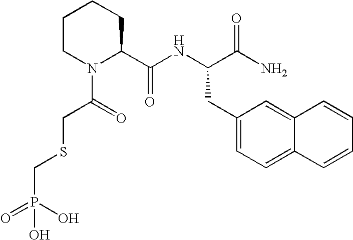 | {2-[2-(1-Carbamoyl-2-naphthalen-2-yl-ethylcarbamoyl)-piperidin-1-yl]-2-oxo-ethylsulfanylmethyl}-phosphonic acid | C22H28N3O6PS | 493.51 | 494.1 |
| 29 | 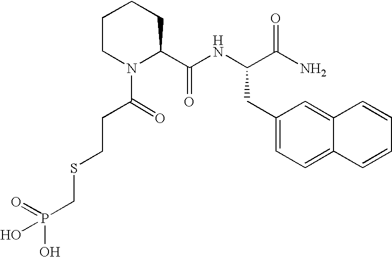 | {3-[2-(1-Carbamoyl-2-naphthalen-2-yl-ethylcarbamoyl)-piperidin-1-yl]-3-oxo-propylsulfanylmethyl}-phosphonic acid | C23H30N3O6PS | 507.54 | 508.3 |
| 30 | 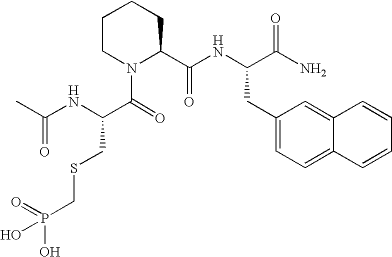 | {2-Acetylamino-3-[2-(1-carbamoyl-2-naphthalen-2-yl-ethylcarbamoyl)-piperidin-1-yl]-3-oxo-propylsulfanylmethyl}-phosphonic acid | C25H33N4O7PS | 564.59 | 565.1 |
| 31 | 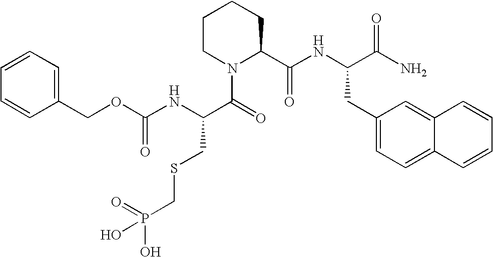 | {2-Benzyloxycarbonylamino-3-[2-(1-carbamoyl-2-naphthalen-2-yl-ethylcarbamoyl)-piperidin-1-yl]-3-oxo-propylsulfanylmethyl}-phosphonic acid | C31H37N4O8PS | 656.69 | 657.2 |
| 32 | 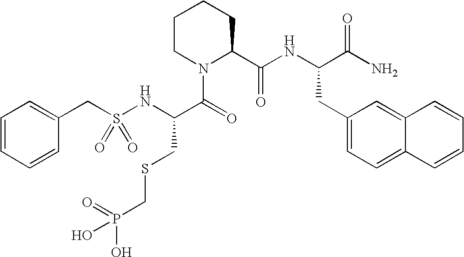 | {3-[2-(1-Carbamoyl-2-naphthalen-2-yl-ethylcarbamoyl)-piperidin-1-yl]-3-oxo-2-phenylmethane-sulfonylaminopropylsulfanyl-methyl}-phosphonic acid | C30H37N4O8PS2 | 676.74 | 677.4 |
| 33 | 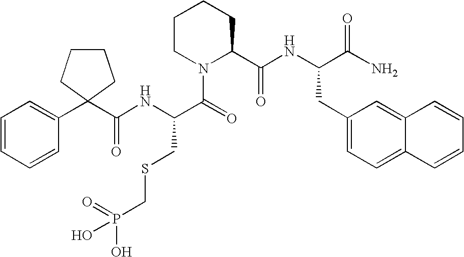 | {3-[2-(1-Carbamoyl-2-naphthalen-2-yl-ethylcarbamoyl)-piperidin-1-yl]-3-oxo-2-[(1-phenyl-cyclopentanecarbonyl)-amino]-propylsulfanylmethyl}-phosphonicacid | C35H43N4O7PS | 694.78 | 695.6 |
| 34 | 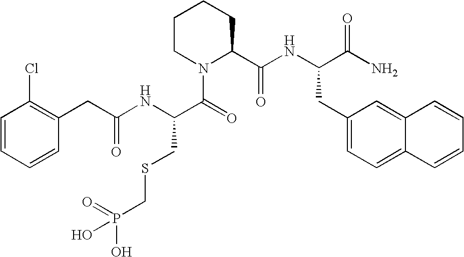 | {3-[2-(1-Carbamoyl-2-naphthalen-2-yl-ethylcarbamoyl)-piperidin-1-yl]-2-[2-(2-chloro-phenyl)-acetylamino]-3-oxo-propylsulfanylmethyl}-phosphonicacid | C31H36ClN4O7PS | 675.13 | 675.6 |
| 35 | 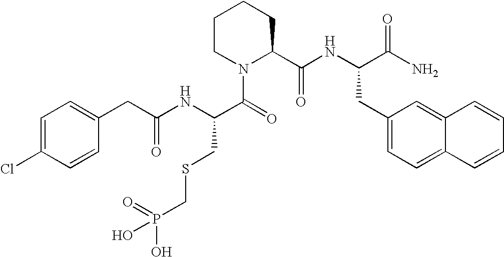 | {3-[2-(1-Carbamoyl-2-naphthalen-2-yl-ethylcarbamoyl)-piperidin-1-yl]-2-[2-(4-chloro-phenyl)-acetylamino]-3-oxo-propylsulfanylmethyl}-phosphonicacid | C31H36ClN4O7PS | 675.13 | 675.3 |
| 36 | 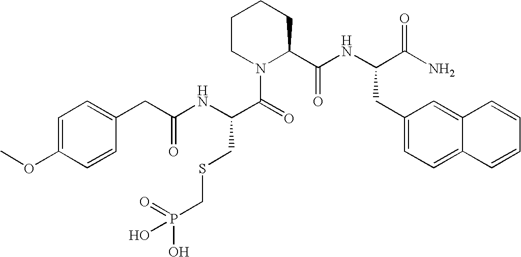 | {3-[2-(1-Carbamoyl-2-naphthalen-2-yl-ethylcarbamoyl)-piperidin-1-yl]-2-[2-(4-methoxy-phenyl)-acetylamino]-3-oxo-propylsulfanylmethyl}-phosphonicacid | C32H39N4O8PS | 670.71 | 671.5 |
| 37 | 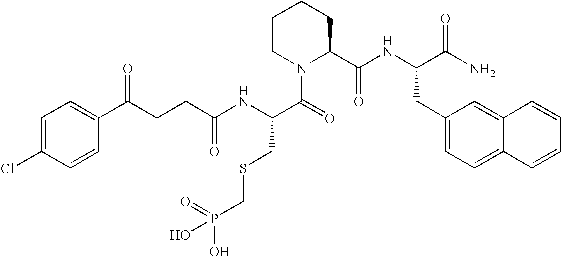 | {3-[2-(1-Carbamoyl-2-naphthalen-2-yl-ethylcarbamoyl)-piperidin-1-yl]-2-[4-(4-chloro-phenyl)-4-oxo-butyrylamino]-3-oxo-propylsulfanylmethyl}-phosphonicacid | C33H38ClN4O8PS | 717.17 | 717.3 |
| 38 | 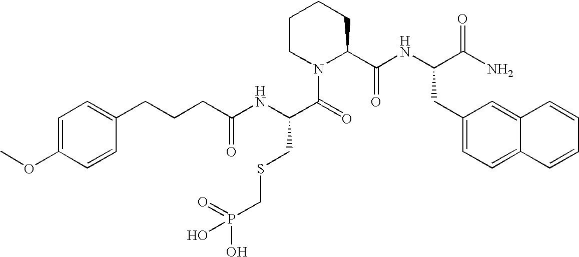 | {3-[2-(1-Carbamoyl-2-naphthalen-2-yl-ethylcarbamoyl)-piperidin-1-yl]-2-[4-(4-methoxy-phenyl)-butyrylamino]-3-oxo-propylsulfanylmethyl}-phosphonicacid | C34H43N4O8PS | 698.77 | 699.2 |
| 39 | 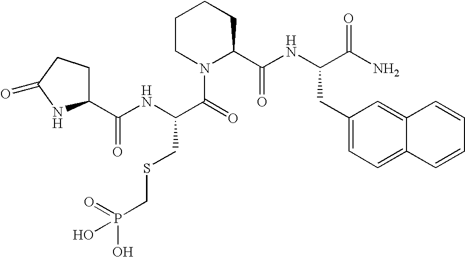 | {3-[2-(1-Carbamoyl-2-naphthalen-2-yl-ethylcarbamoyl)-piperidin-1-yl]-3-oxo-2-[(5-oxo-pyrrolidine-2-carbonyl)-amino]-propylsulfanyl-methyl}-phosphonic acid | C28H36N5O8PS | 633.65 | 634.1 |
| 40 | 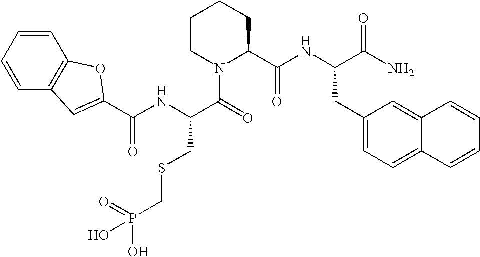 | {2-[(Benzofuran-2-carbonyl)-amino]-3-[2-(1-carbamoyl-2-naphthalen-2-yl-ethylcarbamoyl)-piperidin-1-yl]-3-oxo-propylsulfanylmethyl}-phosphonicacid | C32H35N4O8PS | 666.68 | 667.4 |
| 41 | 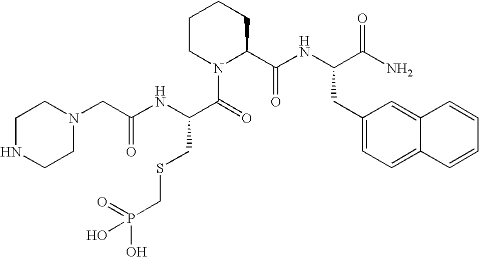 | [3-[2-(1-Carbamoyl-2-naphthalen-2-yl-ethylcarbamoyl)-piperidin-1-yl]-3-oxo-2-(2-piperazin-1-yl-acetylamino)-propylsulfanyl-methyl]-phosphonic acid | C29H41N6O7PS | 648.71 | 649.6 |
| 42 | 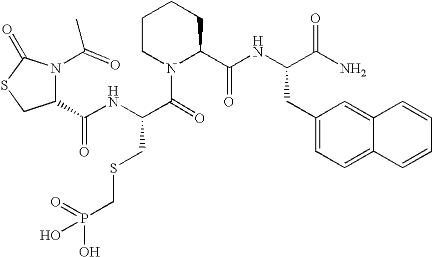 | {2-[(3-Acetyl-2-oxo-thiazolidine-4-carbonyl)-amino]-3-[2-(1-carbamoyl-2-naphthalen-2-yl-ethylcarbamoyl)-piperidin-1-yl]-3-oxo-propylsulfanylmethyl}-phosphonic acid | C29H36N5O9PS2 | 693.73 | 694.4 |
| 43 | 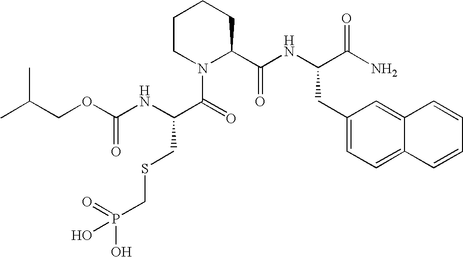 | {3-[2-(1-Carbamoyl-2-naphthalen-2-yl-ethylcarbamoyl)-piperidin-1-yl]-2-isobutoxycarbonylamino-3-oxo-propylsulfanylmethyl}-phosphonic acid | C28H39N4O8PS | 622.67 | 623.2 |
| 44 | 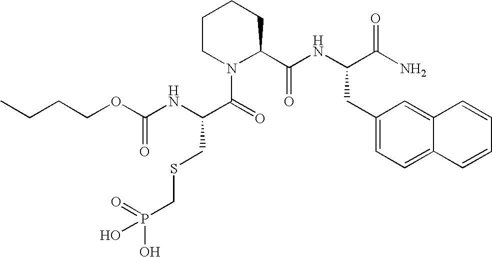 | {2-Butoxycarbonylamino-3-[2-(1-carbamoyl-2-naphthalen-2-yl-ethylcarbamoyl)-piperidin-1-yl]-3-oxo-propylsulfanylmethyl}-phosphonic acid | C28H39N4O8PS | 622.67 | 623.6 |
| 45 | 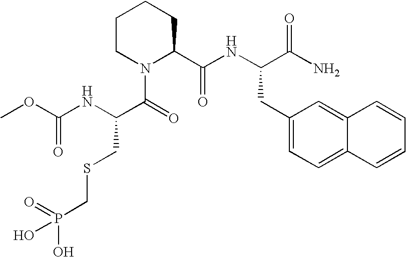 | {3-[2-(1-Carbamoyl-2-naphthalen-2-yl-ethylcarbamoyl)-piperidin-1-yl]-2-methoxycarbonylamino-3-oxo-propylsulfanylmethyl}-phosphonic acid | C25H33N4O8PS | 580.59 | 581.1 |
| 46 | 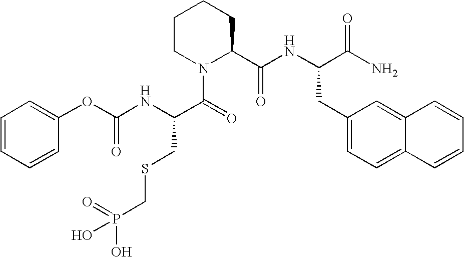 | {3-[2-(1-Carbamoyl-2-naphthalen-2-yl-ethylcarbamoyl)-piperidin-1-yl]3-oxo-2-phenoxycarbonylamino-propylsulfanylmethyl}-phosphonicacid | C30H35N4O8PS | 642.66 | 643.6 |
| 47 | 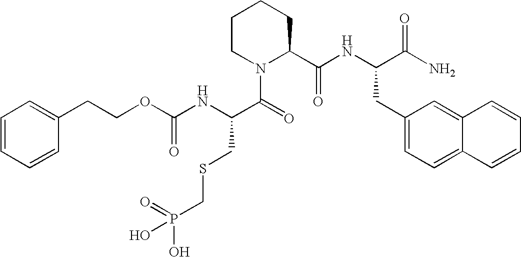 | {3-{2-(1-Carbamoyl-2-naphthalen-2-yl-ethylcarbamoyl)-piperidin-1-yl]-3-oxo-2-phenethyloxycarbonyl-aminopropylsulfanylmethyl}-phosphonic acid | C32H39N4O8PS | 670.71 | 671.5 |
| 48 | 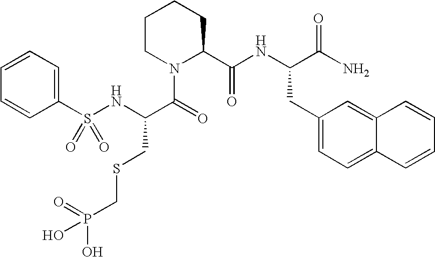 | {2-Benzenesulfonylamino-3-[2-(1-carbamoyl-2-naphthalen-2-yl-ethylcarbamoyl)-piperidin-1-yl]-3-oxo-propylsulfanylmethyl}-phosphonic acid | C29H35N4O8PS2 | 662.71 | 663.3 |
| 49 | 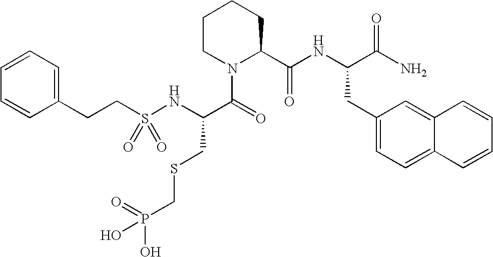 | [3-[2-(1-Carbamoyl-2-naphthalen-2-yl-ethylcarbamoyl)-piperidin-1-yl]-3-oxo-2-(2-phenyl-ethanesulfonylamino)-propylsulfanylmethyl]-phosphonicacid | C31H39N4O8PS2 | 690.77 | 691.4 |
| 50 | 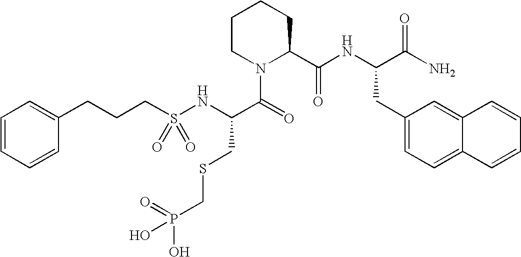 | [3-[2-(1-Carbamoyl-2-naphthalen-2-yl-ethylcarbamoyl)-piperidin-1-yl]-3-oxo-2-(3-phenyl-propane-1-sulfonylamino)-propylsulfanylmethyl]-phosphonicacid | C32H41N4O8PS2 | 704.79 | 705.2 |
| 51 | 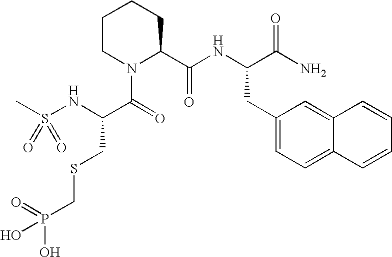 | {3-[2-(1-Carbamoyl-2-naphthalen-2-yl-ethylcarbamoyl)-piperidin-1-yl]-2-methanesulfonylamino-3-oxo-propylsulfanylmethyl}-phosphonic acid | C24H33N4O8PS2 | 600.64 | 601.2 |
| 52 | 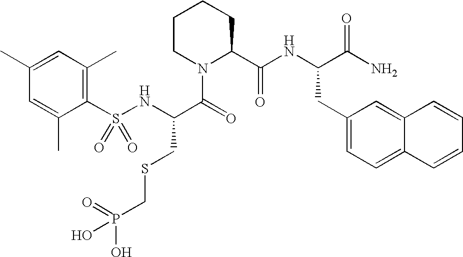 | [3-[2-(1-Carbamoyl-2-naphthalen-2-yl-ethylcarbamoyl)-piperidin-1-yl]-3-oxo-2-(2.4.6-trimethyl-benzenesulfonylamino)-propylsulfanylmethyl]-phosphonicacid | C32H41N4O8PS2 | 704.79 | 705.2 |
| 53 | 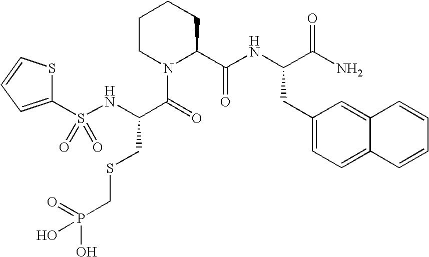 | [3-[2-(1-Carbamoyl-2-naphthalen-2-yl-ethylcarbamoyl)-piperidin-1-yl]-3-oxo-2-(thiophene-2-sulfonylamino)propylsulfanyl-methyl]-phosphonic acid | C27H33N4O8PS3 | 668.74 | 669.5 |
| 54 | 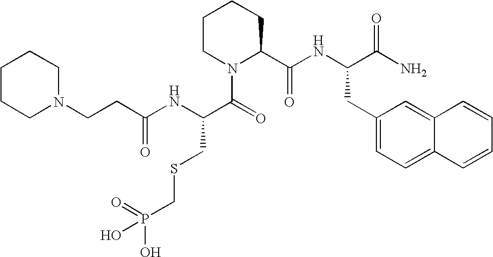 | [3-[2-(1-Carbamoyl-2-naphthalen-2-yl-ethylcarbamoyl)-piperidin-1-yl]-3-oxo-2-(3-piperidin-1-yl-propionylamino)-propylsulfanylmethyl]-phosphonicacid | C31H44N5O7PS | 661.75 | 662.5 |
| 55 | 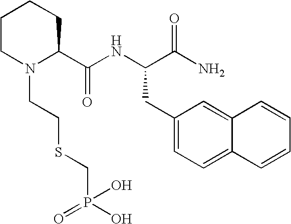 | {2-[2-(1-Carbamoyl-2-naphthalen-2-yl-ethylcarbamoyl)-piperidin-1-yl]-ethylsulfanylmethyl}-phosphonic acid | C22H30N3O5PS | 479.53 | 480.4 |
| 56 | 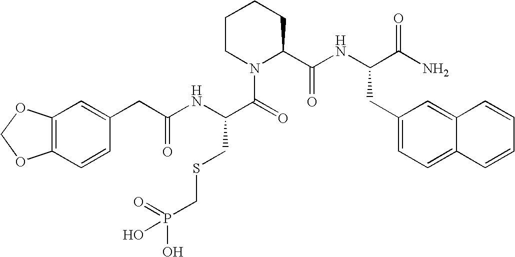 | {2-(2-Benzo[1.3]dioxol-5-yl-acetylamino)-3-[2-(1-carbamoyl-2-naphthalen-2-yl-ethylcarbamoyl)-piperidin-1-yl]-3-oxo-propylsulfanylmethyl}-phosphonic acid | C32H37N4O9PS | 684.70 | 685.6 |
| 57 | 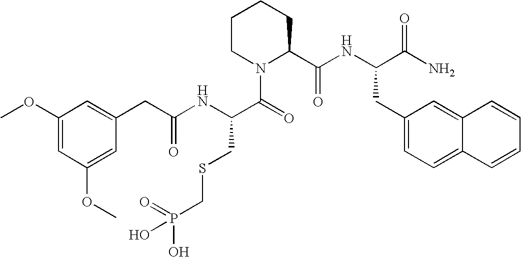 | {3-[2-(1-Carbamoyl-2-naphthalen-2-yl-ethylcarbamoyl)-piperidin-1-yl]-2-[2-(3.5-dimethoxy-phenyl)-acetylamino]-3-oxo-propylsulfanylmethyl}-phosphonicacid | C33H41N4O9PS | 700.74 | 701.3 |
| 58 | 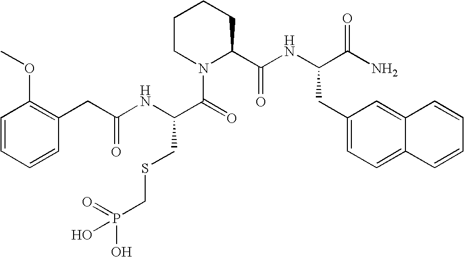 | {3-[2-(1-Carbamoyl-2-naphthalen-2-yl-ethylcarbamoyl)-piperidin-1-yl]-2-[2-(2-methoxy-phenyl)-acetylamino]-3-oxo-propylsulfanylmethyl}-phosphonicacid | C32H39N4O8PS | 670.71 | 671.5 |
| 59 | 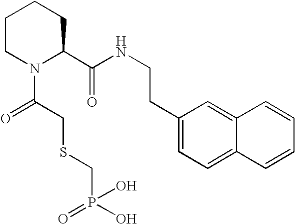 | {2-[2-(2-Naphthalen-2-yl-ethylcarbamoyl)-piperidin-1-yl]-2-oxo-ethylsulfanylmethyl}-phosphonic acid | C21H27N2O5PS | 450.49 | 451.1 |
| 60 | 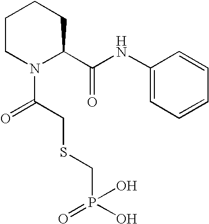 | [2-Oxo-2-(2-phenylcarbamoyl-piperidin-1-yl)-ethylsulfanylmethyl]-phosphonicacid | C36H48N4O10P2S2 | 372.38 | 373.0 |
| 61 | 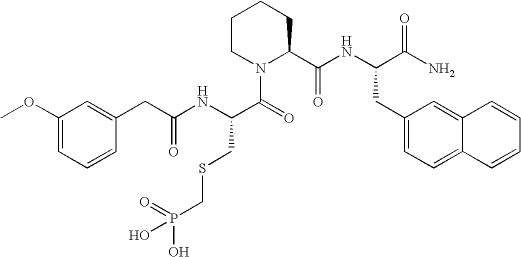 | {3-[2-(1-Carbamoyl-2-naphthalen-2-yl-ethylcarbamoyl)-piperidin-1-yl]-2-[2-(3-methoxy-phenyl)-acetylamino]-3-oxo-propylsulfanylmethyl}-phosphonicacid | C32H39N4O8PS | 670.71 | 671.5 |
| 62 | 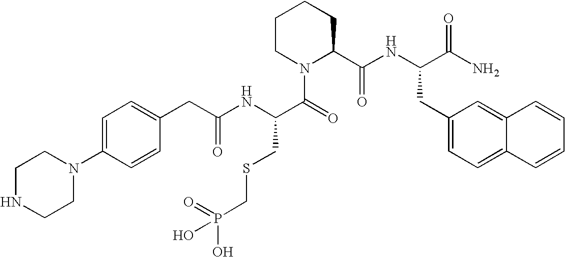 | {3-[2-(1-Carbamoyl-2-naphthalen-2-yl-ethylcarbamoyl)-piperidin-1-yl]-3-oxo-2-[2-(4-piperazin-1-yl-phenyl)-acetylamino]-propylsulfanylmethyl}-phosphonicacid | C35H45N6O7PS | 724.81 | 725.3 |
| 63 | 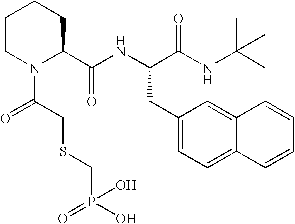 | {2-[2-(1-tert-Butylcarbamoyl-2-naphthalen-2-yl-ethylcarbamoyl)-piperidin-1-yl]-2-oxo-ethylsulfanylmethyl}-phosphonicacid | C26H36N3O6PS | 549.62 | 550.5 |
| 64 | 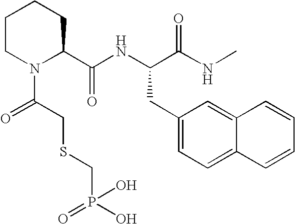 | {2-[2-(1-Methylcarbamoyl-2-naphthalen-2-yl-ethylcarbamoyl)-piperidin-1-yl]-2-oxo-ethylsulfanylmethyl}-phosphonicacid | C23H30N3O6PS | 507.54 | 508.1 |
| 65 | 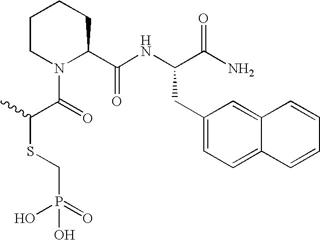 | {2-[2-(1-Carbamoyl-2-naphthalen-2-yl-ethylcarbamoyl)-piperidin-1-yl]-1-methyl-2-oxo-ethylsulfanylmethyl}-phosphonicacid | C23H30N3O6PS | 507.54 | 508.1 |
| 66 | 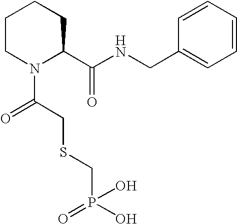 | [2-(2-Benzylcarbamoyl-piperidin-1-yl)-2-oxo-ethylsulfanylmethyl]-phosphonic acid | Cl6H23N2O5PS | 386.40 | 387.1 |
| 67 | 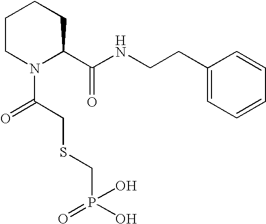 | [2-Oxo-2-(2-phenethylcarbamoyl-piperidin-1-yl)-ethylsulfanylmethyl]-phosphonicacid | C17H25N2O5PS | 400.43 | 401.1 |
| 68 | 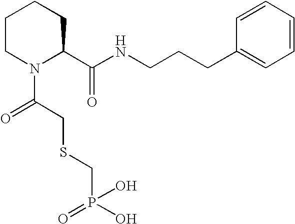 | {2-Oxo-2-[2-(3-phenyl-propylcarbamoyl)-piperidin-1-yl]-ethylsulfanylmethyl}-phosphonicacid | C18H27N2O5PS | 414.46 | 415.3 |
| 69 | 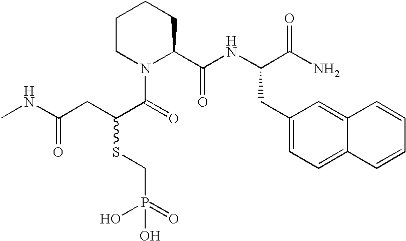 | {2-[2-(1-Carbamoyl-2-naphthalen-2-yl-ethylcarbamoyl)-piperidin-1-yl]-1-methylcarbamoylmethyl-2-oxo-ethylsulfanylmethyl}-phosphonic acid | C25H33N4O7PS | 564.59 | 565.6 |
| 70 | 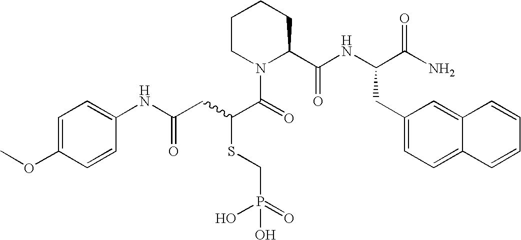 | {2-[2-(1-Carbamoyl-2-naphthalen-2-yl-ethylcarbamoyl)-piperidin-1-yl]-1-[(4-methoxy-phenyl-carbamoyl)-methyl]-2-oxo-ethylsulfanylmethyl}-phosphonicacid | C31H37N4O8PS | 656.69 | 657.1 |
| 71 | 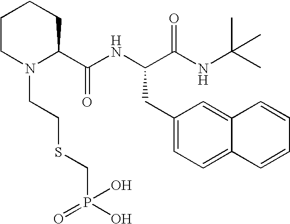 | {2-[2-(1-tert-Butylcarbamoyl-2-naphthalen-2-yl-ethylcarbamoyl)-piperidin-1-yl]-ethylsulfanylmethyl}-phosphonicacid | C26H38N3O5PS | 535.6 | 536.5 |
| 72 | 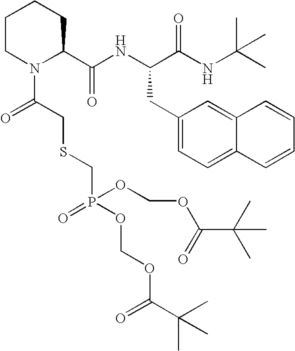 | 2,2-Dimethyl-propionic acid {2-[2-(1-tert-butylcarbamoyl-2-naphthalen-2-yl-ethylcarbamoyl)-piperidin-1-yl]-2-oxo-ethylsulfanylmethyl}-(2,2-dimethyl-propionyloxymethoxy)-phosphinoyloxymethyl ester | C38H56N3O10PS | 777.9 | 778.5 |
| 73 | 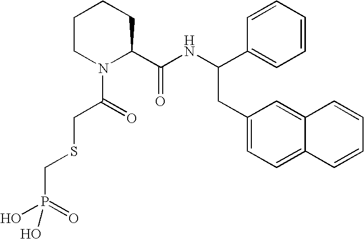 | {2-[2-(2-Naphthalen-2-yl-1-phenyl-ethylcarbamoyl)-piperidin-1-yl]-2-oxo-ethylsulfanylmethyl}-phosphonic acid | C27H31N2O5PS | 526.6 | 527.7 |
| 74 | 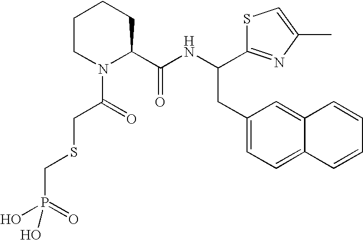 | (2-{2-[1-(4-Methyl-thiazol-2-yl)-2-naphthalen-2-yl-ethylcarbamoyl]-piperidin-1-yl}-2-oxo-ethylsulfanylmethyl)-phosphonic acid | C25H30N3O5PS2 | 547.6 | 548.0 |
| 75 | 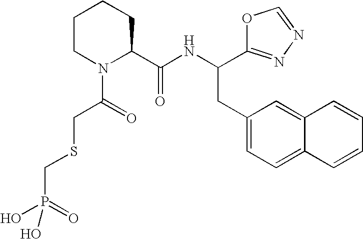 | {2-[2-(2-Naphthalen-2-yl-1-[1,3,4]oxadiazol-2-yl-ethylcarbamoyl)-piperidin-1-yl]-2-oxo-ethylsulfanylmethyl}-phosphonic acid | C23H27N4O6PS | 518.5 | 519.0 |
| 76 | 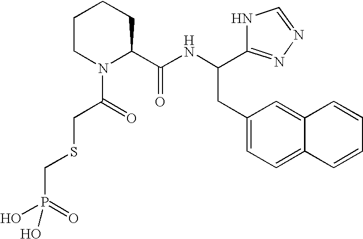 | (2-{2-[2-Naphthalen-2-yl-1-(4H-[1,2,4]triazol-3-yl)-ethylcarbamoyl]-piperidin-1-yl}-2-oxo-ethylsulfanylmethyl)-phosphonic acid | C23H28N5O5PS | 517.5 | 518.1 |
| 77 | 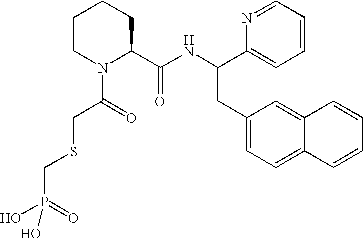 | {2-[2-(2-Naphthalen-2-yl-1-pyridin-2-yl-ethylcarbamoyl)-piperidin-1-yl]-2-oxo-ethylsulfanylmethyl}-phosphonicacid | C26H30N3O5PS | 527.6 | 528.4 |

The problem underlying the present invention is also solved by the technical teaching according to the attached independent claims. Preferred embodiments thereof may be taken from the dependent claims.

The invention is now further illustrated by reference to the following examples from which further advantages, features and embodiments may be taken. It is understood that these examples are given for purpose of illustration only and not for purpose of limitation. All references cited herein are incorporated by reference.

### EXAMPLE 1

### Material and Methods

In order that the invention herein described may be more fully understood, the following detailed description is set forth. As used herein, the following abbreviations are used:
Ac is acetyl,
ACN is acetonitrile,
Boc is tert-butyloxycarbonyl;
Bta is 3-benzo[b]thiophen-3-yl-alanine,
d is day,
DCM is dichloromethane;
DIEA is N,N-diisopropylethylamine;
DMF is N,N-dimethylformamide;
DMSO is N,N-dimethylsulfoxide;
Fmoc is 9H-fluoren-9-ylmethoxycarbonyl;
HMDS is 1,1,1,3,3,3-hexamethyldisilazane;
HOBt is 1-hydroxybenzotriazole, monohydrate;
HPLC is high performance liquid chromatography;
h is hour;
mL is milliliter;
THF is tetrahydrofuran;
TFA is trifluoro-acetic acid;
TMSBr is bromo-trimethyl-silane;
WSC is 1-(3-dimethylaminopropyl)-3-ethylcarbodiimide hydrochloride.

### Method A: Fmoc Deprotection on Solid Support

Resin-bound and N-Fmoc-protected amine (80 mg, 40 μmol) was washed with DMF (5×1 mL) and agitated with a solution of 20% piperidine in DMF (2×1 mL) for 10 and 20 min. The resin was then filtered and washed with DMF (5×1 mL)

### Method B: Amide Bond Formation on Solid Support

Resin-bound amine (80 mg, 40 μmol) was washed with DMF (5×1 mL) and suspended in a solution of the desired carboxylic acid (0.20 mmol), O-(7-azabenzotriazol-1-yl)-N,N,N′,N′-tetramethyluronium hexafluorophosphate (76 mg, 0.20 mmol), 7-aza-1-hydroxybenzotriazole (27 mg, 0.20 mmol), and collidine (0.26 mL, 0.24 g, 20 mmol) in DMF (0.8 mL). After agitation for 12 h at room temperature, the solution was drained and the resin was washed with DMF (7×1 mL).

### Method C: Acetylation of Resin-Bound Amine

Resin-bound amine (80 mg, 40 μmol) was washed with DMF (5×1 mL) and suspended in a 7:2:1 solution of DMF, DIEA, and acetic anhydride. After agitation for 20 min at room temperature, the solution was drained and the resin was washed with DMF (7×1 mL).

Method D: Cleavage from Resin

The resin was washed with DCM (5×1 mL) and subsequently treated with a 2% TFA solution in DCM (4×1 mL) under agitation for 30 min in each case. The solvent of the combined filtrates was removed in vacuo to give the crude product.

### Method E: Deprotection of Phosphonic Acid Diethyl Ester

HMDS (0.40 mL, 0.31 g, 1.9 mmol) and subsequently TMSBr (0.70 mL, 0.81 g, 5.3 mmol) were added dropwise to a stirred solution of phosphonic acid diethyl ester (40 μmol) in anhydrous DCM (2 mL) at 0° C. After stirring for 30 min at 0° C., the reaction mixture was allowed to warm to room temperature and stirred for an additional 3.5 h. The reaction mixture was then concentrated in vacuo and the residue was redissolved in a mixture of water (1 mL) and acetonitrile (2 mL). Evaporation of the solvent in vacuo gave the crude product.

### EXAMPLE 2

### Synthesis of {2-(2-acetylamino-3-benzo[b]thiophen-3-yl-propionylamino)-3-[2-(1-carbamoyl-2-naphthalen-2-yl-ethylcarbamoyl)-piperidin-1-yl]-3-oxo-propoxymethyl}-phosphonic acid

### A. 2-Amino-3-naphthalen-2-yl-propionamide Resin

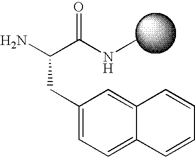

9-Fmoc-amino-xanthen-3-yloxy-Merrifield resin (0.50 mmol/g, 80 mg, purchased from NovaBiochem) was deprotected according to method A, 2-(9H-fluoren-9-ylmethoxycarbonylamino)-3-naphthalen-2-yl-propionic acid (87 mg, 0.20 mmol) was attached to the resin according to method B and subsequently deprotected according to method A to give the title compound.

### B. Piperidine-2-carboxylic acid (1-carbamoyl-2-naphthalen-2-yl-ethyl)-amide Resin

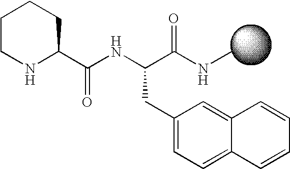

Piperidine-1,2-dicarboxylic acid 1-(9H-fluoren-9-ylmethyl) ester (70 mg, 0.20 mmol) was coupled to 2-amino-3-naphthalen-2-yl-propionamide resin (80 mg, 40 μmol) according to method B and subsequently deprotected according method A to give the title compound.

### C. 3-(Diethoxy-phosphorylmethoxy)-2-(9H-fluoren-9-ylmethoxycarbonylamino)-propionic acid

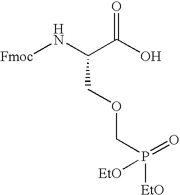

NaH (60% dispersion in mineral oil, 0.50 g, 13 mmol) was washed with anhydrous hexane (2×3 mL) and suspended in anhydrous DMF (15 mL). A solution of 2-tert-butoxycarbonylamino-3-hydroxy-propionic acid (1.2 g, 5.7 mmol) in anhydrous DMF (10 mL) was added dropwise to the stirred suspension at 0° C. over 15 min. The reaction mixture was allowed to warm to room temperature and stirred for an additional 50 min. The reaction mixture was cooled to 0° C. and subsequently a solution of trifluoro-methanesulfonic acid diethoxy-phosphorylmethyl ester (1.7 g, 5.7 mmol) in DMF (10 mL) was added dropwise over 15 min. After stirring for 40 min at 0° C., the reaction was quenched by the addition of acetic acid (3 mL). The reaction mixture was concentrated in vacuo, the residue was partitioned between ethyl acetate (100 mL) and aqueous Na₂CO₃ (1.0 M, 20 mL), and the organic layer was extracted with 1.0 M aqueous Na₂CO₃ (3×3 mL). The combined aqueous layers were acidified with 6 M aqueous HCl to pH 1 and extracted with ethyl acetate (10×15 mL). The combined organic layers were dried over Na₂SO₄, filtered, and concentrated in vacuo. The crude 2-tert-butoxycarbonylamino-3-(diethoxy-phosphorylmethoxy)-propionic acid was dissolved in a mixture of DCM (20 mL), water (0.20 mL), and TFA (4.0 mL). After stirring for 30 min at room temperature the reaction mixture was concentrated in vacuo. The residue (0.88 g) was dissolved in DMF (15 mL). DIEA (0.85 mL, 0.67 g, 5.2 mmol) and a solution of carbonic acid 2,5-dioxo-pyrrolidin-1-yl ester 9H-fluoren-9-ylmethyl ester (0.67 g, 2.0 mmol) in DMF (5 mL) were added dropwise to the stirred reaction mixture at 0° C. The reaction mixture was allowed to warm to room temperature, stirred for an additional 90 min and subsequently the solvent was removed in vacuo. The residue was purified by flash chromatography over silica gel (elution with 5% acetic acid in ethyl acetate) and subsequently by solid phase extraction over Bondesil-ENV, 125UM (purchased from Varian, elution with a gradient from 0% to 90% acetonitrile in water) to give the title compound as a colorless oil. MS (m/z): 478.3 [M+H⁺].

### D. {2-(2-Acetylamino-3-benzo[b]thiophen-3-yl-propionylamino)-3-[2-(1-carbamoyl-2-naphthalen-2-yl-ethylcarbamoyl)-piperidin-1-yl]-3-oxo-propoxymethyl}-phosphonic acid

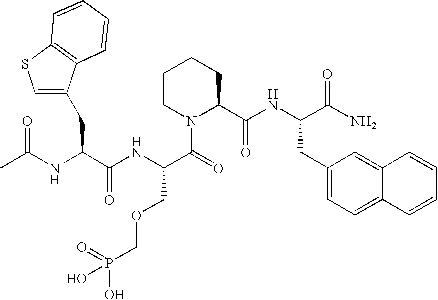

3-(Diethoxy-phosphorylmethoxy)-2-(9H-fluoren-9-ylmethoxycarbonylamino)-propionic acid (95 mg, 0.20 mmol) was coupled to piperidine-2-carboxylic acid (1-carbamoyl-2-naphthalen-2-yl-ethyl)-amide resin (80 mg, 40 μmol) according to method B and subsequently deprotected according to method A. 3-Benzo[b]thiophen-3-yl-2-(9H-fluoren-9-ylmethoxycarbonylamino)-propionic acid (89 mg, 0.20 mmol) was coupled to the resin according to method B, subsequently deprotected according to method A, acetylated according to method C, and finally the resin-bound compound was cleaved according to method D. The crude phosphonic acid diethyl ester was deprotected according to method E and subsequently purified by reversed phase HPLC to give the title compound as a white solid. MS (m/z): 752.3 [M+H⁺].

### EXAMPLE 3

### Synthesis of {2-(2-acetylamino-3-benzo[b]thiophen-3-yl-propionylamino)-3-[2-(1-carbamoyl-2-naphthalen-2-yl-ethylcarbamoyl)-piperidin-1-yl]-3-oxo-propylsulfanylmethyl}-phosphonic acid

### A. 3-(Diethoxy-phosphorylmethylsulfanyl)-2-(9H-fluoren-9-ylmethoxycarbonylamino)-propionic acid

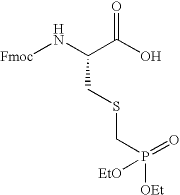

Cysteine (1.0 g, 8.3 mmol) was dissolved in a mixture of water (40 mL) and dioxane (10 mL). Iodomethyl-phosphonic acid diethyl ester (3.0 g, 11 mmol) and Na₂CO₃ (2.0 g, 19 mmol) were added to the reaction mixture. After stirring for 6 h at 50° C., the reaction mixture was cooled to 0° C. and a solution of carbonic acid 2,5-dioxo-pyrrolidin-1-yl ester 9H-fluoren-9-ylmethyl ester (4.2 g, 12 mmol) in dioxane (16 mL) was added dropwise. The reaction was allowed to warm to room temperature. After stirring for 12 h, the reaction mixture was concentrated in vacuo and the residue was partitioned between ethyl acetate (30 mL) and water (50 mL). The aqueous layer was extracted with ethyl acetate (2×30 mL), acidified with 6 M aqueous HCl to pH 1, and extracted with ethyl acetate (5×20 mL). The combined organic layers were dried over Na₂SO₄, filtered, and concentrated in vacuo. The residue was purified by flash chromatography over silica gel (elution with DCM/ethyl acetate/acetic acid 90:10:5) to give the title compound as a colorless oil. MS (m/z): 494.2 [M+H⁺].

### B. {2-Amino-3-[2-(1-carbamoyl-2-naphthalen-2-yl-ethylcarbamoyl)-piperidin-1-yl]-3-oxo-propylsulfanylmethyl}-phosphonic acid Resin

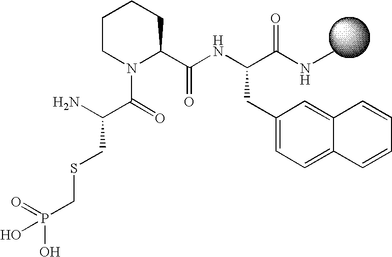

3-(Diethoxy-phosphorylmethylsulfanyl)-2-(9H-fluoren-9-ylmethoxycarbonylamino)-propionic acid (99 mg, 0.20 mmol) was coupled to piperidine-2-carboxylic acid (1-carbamoyl-2-naphthalen-2-yl-ethyl)-amide resin (80 mg, 40 μmol) according to method B and subsequently deprotected according to method A to give the title compound.

### C. {2-(2-Acetylamino-3-benzo[b]thiophen-3-yl-propionylamino)-3-[2-(1-carbamoyl-2-naphthalen-2-yl-ethylcarbamoyl)-piperidin-1-yl]-3-oxo-propylsulfanylmethyl}-phosphonic acid

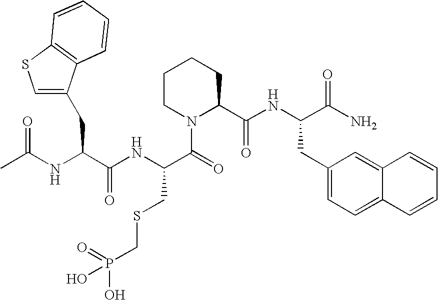

3-Benzo[b]thiophen-3-yl-2-(9H-fluoren-9-ylmethoxycarbonylamino)-propionic acid (89 mg, 0.20 mmol) was coupled to {2-amino-3-[2-(1-carbamoyl-2-naphthalen-2-yl-ethylcarbamoyl)-piperidin-1-yl]-3-oxo-propylsulfanylmethyl}-phosphonic acid resin (80 mg, 40 μmol) according to method B, subsequently deprotected according to method A, and acetylated according to method C. The resin-bound compound was cleaved according to method D. The crude phosphonic acid diethyl ester was deprotected according to method E and subsequently purified by reversed phase HPLC to give the title compound as a white solid. MS (m/z): 768.3 [M+H⁺].

### EXAMPLE 4

### {3-[2-(1-Carbamoyl-2-naphthalen-2-yl-ethylcarbamoyl)-piperidin-1-yl]-2-[3-(3,4-dichloro-benzylsulfanyl)-propionylamino]-3-oxo-propylsulfanylmethyl}-phosphonic acid

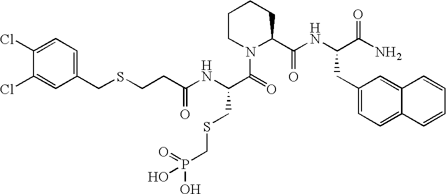

3-(3,4-Dichloro-benzylsulfanyl)-propionic acid (53 mg, 0.20 mmol) was coupled to {2-amino-3-[2-(1-carbamoyl-2-naphthalen-2-yl-ethylcarbamoyl)-piperidin-1-yl]-3-oxo-propylsulfanylmethyl}-phosphonic acid resin (80 mg, 40 μmol) according to method B, subsequently deprotected according to method A, and the resin-bound compound was cleaved according to method D. The crude phosphonic acid diethyl ester was deprotected according to method E and subsequently purified by reversed phase HPLC to give the title compound as a white solid. MS (m/z): 769.2 [M+H⁺].

### EXAMPLE 5

### Synthesis of 2-(2-acetylamino-3-benzo[b]thiophen-3-yl-propionylamino)-3-[2-(1-carbamoyl-2-naphthalen-2-yl-ethylcarbamoyl)-piperidin-1-yl]-propylsulfanylmethyl}-phosphonic acid

### A. (2-tert-Butoxycarbonylamino-3-hydroxy-propylsulfanylmethyl)-phosphonic acid diethyl ester

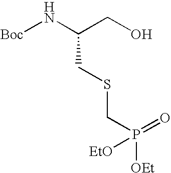

Cysteine (0.50 g, 4.1 mmol) was dissolved in a mixture of water (20 mL) and dioxane (5 mL). Iodomethyl-phosphonic acid diethyl ester (1.2 g, 4.3 mmol) and Na₂CO₃ (0.87 g, 8.2 mmol) were added to the reaction mixture. After stirring for 6 h at 50° C., the reaction mixture was cooled to 0° C. and a solution of di-tert-butyl pyrocarbonate (1.4 g, 6.5 mmol) in dioxane (8 mL) was added dropwise. The reaction was allowed to warm to room temperature. After stirring for 12 h, the reaction mixture was concentrated in vacuo and the residue was partitioned between ethyl acetate (20 mL) and water (30 mL). The aqueous layer was extracted with ethyl acetate (2×10 mL), acidified with 6 M aqueous HCl to pH 1, and extracted with ethyl acetate (3×15 mL). The organic layer was dried over Na₂SO₄, filtered, and concentrated in vacuo. The crude 2-tert-butoxycarbonylamino-3-(diethoxy-phosphorylmethylsulfanyl)-propionic acid (0.28 g, 0.76 mmol) was dissolved in anhydrous THF (2.5 mL). 4-Methyl-morpholine (84 μL, 77 mg, 0.76 mmol) and subsequently isobutyl chloroformate (100 μL, 0.10 g, 0.76 mmol) were added dropwise to the stirred reaction mixture at −15° C. After stirring for 1 h at −15° C., the reaction mixture was filtered. A solution of NaBH₄ (42 mg, 1.1 mmol) in water (0.37 mL) was then added in one portion to the stirred filtrate at −15° C. After 30 min at −15° C., the reaction mixture was partitioned between water (1 mL) and ethyl acetate (20 mL). The organic layer was dried over Na₂SO₄, filtered, and concentrated in vacuo. The residue was purified by flash chromatography over silica gel (elution with ethyl acetate/methanol 9:1) to give the title compound as a colorless oil. MS (m/z): 357.9 [M+H⁺].

### B. (2-tert-Butoxycarbonylamino-3-iodo-propylsulfanylmethyl)-phosphonic acid diethyl ester

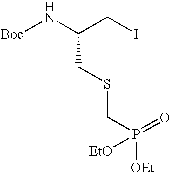

Methanesulfonyl chloride (88 μL, 0.13 g, 1.1 mmol) was added dropwise to a stirred solution of (2-tert-butoxycarbonylamino-3-hydroxy-propylsulfanylmethyl)-phosphonic acid diethyl ester (0.16 g, 0.46 mmol) and Et₃N (0.19 mL, 0.14 g, 1.4 mmol) in DCM (3.2 mL) at 0° C. After stirring for 80 min at 0° C., the reaction mixture was partitioned between concentrated aqueous NaHCO₃ (6.4 mL) and DCM (20 mL). The aqueous layer was extracted with DCM (10 mL) and the combined organic layers were dried over Na₂SO₄, filtered, and concentrated in vacuo. The crude methanesulfonic acid 2-tert-butoxycarbonylamino-3-(diethoxy-phosphorylmethylsulfanyl)-propyl ester (0.15 g, 0.34 mmol) and NaI (0.15 g, 1.0 mmol) was dissolved in acetone (3 mL). After stirring for 3 h at 55° C., the reaction mixture was cooled to room temperature, diluted with ethyl acetate (20 mL), filtered, and concentrated in vacuo. The residue was purified by flash chromatography over silica gel (elution with ethyl acetate) to give the title compound as a yellow oil. MS (m/z): 490.1 [M+Na⁺].

### C. {2-Amino-3-[2-(1-carbamoyl-2-naphthalen-2-yl-ethylcarbamoyl)-piperidin-1-yl]-propylsulfanylmethyl}-phosphonic acid diethyl ester

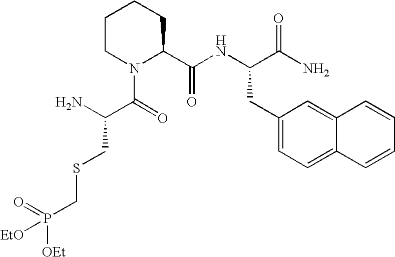

Piperidine-2-carboxylic acid (1-carbamoyl-2-naphthalen-2-yl-ethyl)-amide resin (80 mg, 40 μmol) was washed with DMF (4×1 mL) and suspended in a solution of (2-tert-butoxycarbonylamino-3-iodo-propylsulfanylmethyl)-phosphonic acid diethyl ester (65 mg, 0.14 mmol) and DIEA (48 μL, 36 mg, 0.28 mmol) in DMF (0.22 mL). After agitation for 3 d at 35° C., the solution was drained and the resin was washed with DMF (7×1 mL). The resin-bound product was cleaved according to method D. The residue was treated with a mixture of DCM (1 mL) a TFA (1 mL) for 1 h. The mixture was concentrated in vacuo and purified by reversed phase HPLC to give the title compound as a colorless oil. MS (m/z): 565.5 [M+H⁺].

### D. {2-(2-Acetylamino-3-benzo[b]thiophen-3-yl-propionylamino)-3-[2-(1-carbamoyl-2-naphthalen-2-yl-ethylcarbamoyl)-piperidin-1-yl]-propylsulfanylmethyl}-phosphonic acid

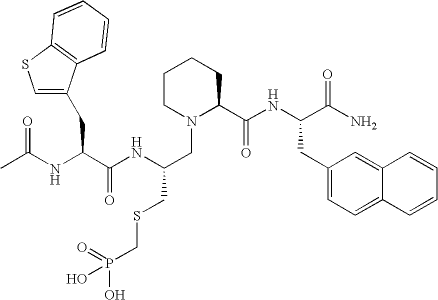

O-(7-azabenzotriazol-1-yl)-N,N,N′,N′-tetramethyluronium hexafluorophosphate (7.9 mg, 21 μmol) was added to a stirred solution of {2-amino-3-[2-(1-carbamoyl-2-naphthalen-2-yl-ethylcarbamoyl)-piperidin-1-yl]-propylsulfanylmethyl}-phosphonic acid diethyl ester (11 mg, 20 μmol), 7-aza-1-hydroxybenzotriazole (2.8 mg, 21 μmol), and DIEA (2.3 μL, 1.7 mg, 14 μmol) in DMF (0.7 mL), followed by the addition of collidine (28 μL, 26 mg, 0.21 mmol) and 2-acetylamino-3-benzo[b]thiophen-3-yl-propionic acid (5.5 mg, 21 μmol). After stirring for 3 h, the reaction mixture was concentrated in vacuo and purified by reversed phase HPLC. The resulting {2-(2-acetylamino-3-benzo[b]thiophen-3-yl-propionylamino)-3-[2-(1-carbamoyl-2-naphthalen-2-yl-ethylcarbamoyl)-piperidin-1-yl]-propylsulfanylmethyl}-phosphonic acid diethyl ester (6 mg) was deprotected according to method E and purified by reversed phase HPLC to give the title compound as a white solid. MS (m/z): 754.6 [M+H⁺].

### EXAMPLE 6

### Synthesis of Ac-Bta-Cys(CH₂—P(O)(OH)₂)—NMeazaAla-2Nal-NH₂

### A. Fmoc-NMeazaAla-Cl

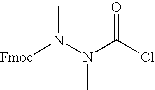

A solution of N,N′-dimethyl-hydrazinecarboxylic acid 9H-fluoren-9-ylmethyl ester (0.16 g, 0.57 mmol) in anhydrous THF (3.5 mL) was added dropwise to a stirred 20% phosgene solution in toluene (1.1 mL) at 0° C. The reaction mixture was then allowed to warm to room temperature and stirred for 1 h. The reaction mixture was concentrated in vacuo, redissolved in ethyl acetate (10 mL), and filtered through a pad of silica gel. The solvent was removed in vacuo to give the title compound as a colorless oil.

### B. Ac-Bta-Cys(CH₂—P(O)(OH)₂)—NMeazaAla-2Nal-NH₂

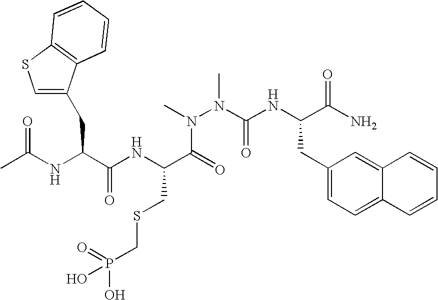

2-Amino-3-naphthalen-2-yl-propionamide resin (80 mg, 40 μmol) was washed with DMF (5×1 mL) and suspended in a solution of Fmoc-NMeazaAla-Cl (55 mg, 0.16 mmol) and DIEA (34 μL, 26 mg, 0.20 mmol) in DMF (0.15 mL). After agitation for 12 h at room temperature, the solution was drained. The resin was washed with DMF (7×1 mL), deprotected according to method A, and washed with anhydrous THF (5×1 mL). 3-(Diethoxy-phosphorylmethylsulfanyl)-2-(9H-fluoren-9-ylmethoxycarbonylamino)-propionic acid (64 mg, 0.13 mmol) and triphosgene (13 mg, 0.044 mmol) were dissolved in anhydrous THF (0.9 mL) and subsequently collidine (48 μL, 44 mg, 0.37 mmol) was added in one portion. After 1 min, the resulting suspension was added to the resin and the mixture was agitated for 50 min at 50° C. The solution was drained and the resin was washed with DMF (7×1 mL) and deprotected according to method A. 3-Benzo[b]thiophen-3-yl-2-(9H-fluoren-9-ylmethoxycarbonylamino)-propionic acid (89 mg, 0.20 mmol) was coupled to the resin-bound amine according to method B, subsequently deprotected according to method A, acetylated according to method C, and the resin-bound compound was cleaved according to method D. The crude phosphonic acid diethyl ester was deprotected according to method E and subsequently purified by reversed phase HPLC to give the title compound as a white solid. MS (m/z): 743.4 [M+H⁺].

### EXAMPLE 7

### Synthesis of {2-[2-(1-carbamoyl-2-naphthalen-2-yl-ethylcarbamoyl)-piperidin-1-yl]-2-oxo-ethylsulfanylmethyl}-phosphonic acid

### A. (Diethoxy-phosphorylmethylsulfanyl)-acetic acid

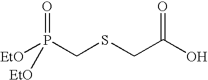

Mercaptoacetic acid sodium salt (0.41 g, 3.6 mmol), iodomethyl-phosphonic acid diethyl ester (1.0 g, 3.6 mmol), and Na₂CO₃ (0.38 g, 3.6 mmol) were dissolved in a mixture of water (17 mL) and dioxane (4.3 mL). After stirring for 20 h at 50° C., the reaction mixture was concentrated in vacuo to a volume of approximately 10 mL. The residue was acidified with 6 M aqueous HCl to pH 1 and extracted with ethyl acetate (3×20 mL). The combined organic layers were dried over Na₂SO₄, filtered, and concentrated in vacuo to give the title compound as a yellow oil. MS (m/z): 242.9 [M+H⁺].

### B. {2-[2-(1-Carbamoyl-2-naphthalen-2-yl-ethylcarbamoyl)-piperidin-1-yl]-2-oxo-ethylsulfanylmethyl}-phosphonic acid

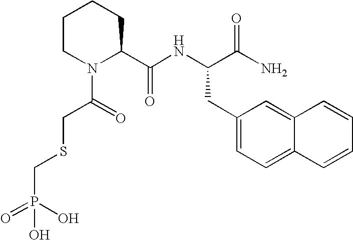

(Diethoxy-phosphorylmethylsulfanyl)-acetic acid (48 mg, 0.20 mmol) was coupled to piperidine-2-carboxylic acid (1-carbamoyl-2-naphthalen-2-yl-ethyl)-amide resin (80 mg, 40 μmol) according to method B and subsequently deprotected according to method A. The resin-bound compound was cleaved according to method D, the resulting crude phosphonic acid diethyl ester was deprotected according to method E and purified by reversed phase HPLC to give the title compound as a white solid. MS (m/z): 494.1 [M+H⁺].

### EXAMPLE 8

### {2-Benzyloxycarbonylamino-3-[2-(1-carbamoyl-2-naphthalen-2-yl-ethylcarbamoyl)-piperidin-1-yl]-3-oxo-propylsulfanylmethyl}-phosphonic acid

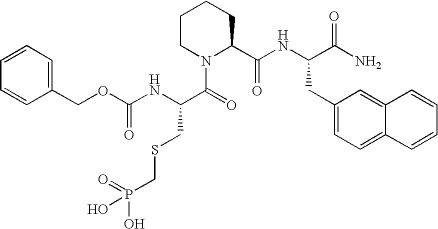

{2-Amino-3-[2-(1-carbamoyl-2-naphthalen-2-yl-ethylcarbamoyl)-piperidin-1-yl]-3-oxo-propylsulfanylmethyl}-phosphonic acid resin (80 mg, 40 μmol) was washed with anhydrous DCM (3×1 mL) and suspended in a solution of benzyl chloroformate (34 μL, 34 mg, 0.20 mmol) in anhydrous DCM (0.7 mL). After agitation for 10 min, a solution of DIEA (41 μL, 31 mg, 0.24 mmol) in DCM (0.6 mL) was added to the reaction mixture in three equal portions over 1 h. The mixture was agitated for an additional 1 h and washed with DCM (5×1 mL). The resin-bound compound was cleaved according to method D. The crude phosphonic acid diethyl ester was deprotected according to method E and purified by reversed phase HPLC to give the title compound as a white solid. MS (m/z): 657.2 [M+H⁺].

### EXAMPLE 9

### {3-[2-(1-Carbamoyl-2-naphthalen-2-yl-ethylcarbamoyl)-piperidin-1-yl]-3-oxo-2-phenylmethanesulfonylamino-propylsulfanylmethyl}-phosphonic acid

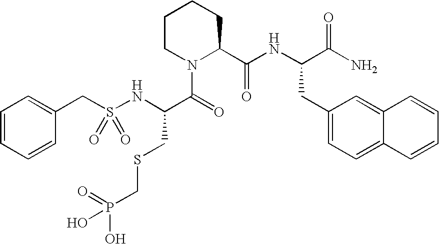

{2-Amino-3-[2-(1-carbamoyl-2-naphthalen-2-yl-ethylcarbamoyl)-piperidin-1-yl]-3-oxo-propylsulfanylmethyl}-phosphonic acid resin (80 mg, 40 μmol) was washed with anhydrous DCM (3×1 mL) and suspended in a solution of phenyl-methanesulfonyl chloride (38 mg, 0.20 mmol) in anhydrous DCM (0.7 mL). After agitation for 10 min, pyridine (0.30 mL) was added to the reaction mixture. The mixture was agitated for an additional 2 h and washed with DCM (5×1 mL). The resin-bound compound was cleaved according to method D. The crude phosphonic acid diethyl ester was deprotected according to method E and purified by reversed phase HPLC to give the title compound as a white solid. MS (m/z): 677.4 [M+H⁺].

### EXAMPLE 10

### Synthesis of {2-[2-(1-carbamoyl-2-naphthalen-2-yl-ethylcarbamoyl)-piperidin-1-yl]-ethylsulfanylmethyl}-phosphonic acid

### A. 1-[2-(Diethoxy-phosphorylmethylsulfanyl)-ethyl]-piperidine-2-carboxylic acid

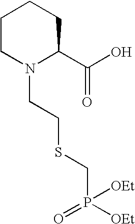

A solution of piperidine-2-carboxylic acid methyl ester (0.11 g, 0.74 mmol) and thiirane (66 μL, 67 mg, 1.1 mmol) in toluene (1 mL) was stirred in a sealed bottle under an argon atmosphere at 110° C. for 17 h. The solvent was removed in vacuo and the residue was dissolved in a mixture of water (3 mL) and dioxane (0.75 mL). Iodomethyl-phosphonic acid diethyl ester (0.20 g, 0.72 mmol) and Na₂CO₃ (78 mg, 0.74 mmol) were added to the reaction mixture. After stirring for 22 h at 55° C., the reaction mixture was allowed to cool to room temperature and a solution of LiOH (75 mg, 3.1 mmol) in water (0.30 mL) was added. The reaction mixture was stirred for 28 h at room temperature and for an additional 2 h at 45° C. The reaction mixture was then concentrated in vacuo and the residue was purified by reversed phase HPLC to give the title compound as a colorless oil. MS (m/z): 340.2 [M+H⁺]

### B. {2-[2-(1-Carbamoyl-2-naphthalen-2-yl-ethylcarbamoyl)-piperidin-1-yl]-ethylsulfanylmethyl}-phosphonic acid

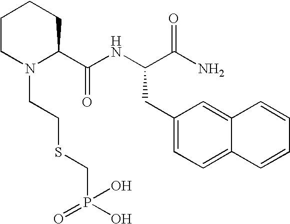

1-[2-(Diethoxy-phosphorylmethylsulfanyl)-ethyl]-piperidine-2-carboxylic acid (24 mg, 70 μmol), HOBt (9.4 mg, 70 μmol), 2-amino-3-naphthalen-2-yl-propionamide (15 mg, 70 μmol), and DIEA (12 μL, 9.1 mg, 70 mmol) were dissolved in DMF (0.3 mL). WSC (13 mg, 70 μmol) was then added in one portion to the vigorously stirred solution. After 2 h at room temperature, the reaction mixture was concentrated in vacuo. The residue was partitioned between ethyl acetate (30 mL) and 1.0 M aqueous Na₂CO₃ (3 mL), and the organic layer was extracted with 1.0 M aqueous Na₂CO₃ (2×2 mL). The combined organic layers were dried over Na₂SO₄, filtered, and concentrated in vacuo. The resulting crude phosphonic acid diethyl ester was deprotected according to method E and subsequently purified by reversed phase HPLC to give the title compound as a white solid. MS (m/z): 480.4 [M+H⁺].

### EXAMPLE 11

### Synthesis of {2-[2-(1-tert-butylcarbamoyl-2-naphthalen-2-yl-ethylcarbamoyl)-piperidin-1-yl]-2-oxo-ethylsulfanylmethyl}-phosphonic acid

### A. 1-[2-(Diethoxy-phosphorylmethylsulfanyl)-acetyl]-piperidine-2-carboxylic acid

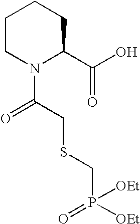

Piperidine-1,2-dicarboxylic acid 1-(9H-fluoren-9-ylmethyl) ester (0.52 g, 1.5 mmol) and trityl chloride resin (0.50 g, 1.5 mmol/g, purchased from NovaBiochem) were suspended in anhydrous DCM (5.9 mL). DIEA (0.37 mL, 0.28 g, 2.2 mmol) was then added and the suspension was agitated for 1 h. The solution was drained and the resin was washed with methanol (2×5 mL), a 9:1 mixture of methanol/DIEA (5 mL), DMF (3×5 mL), and DCM (2×5 mL). The resin was then dried in vacuo. The resulting piperidine-1,2-dicarboxylic acid 1-(9H-fluoren-9-ylmethyl) ester resin (0.20 g) was deprotected according to method A, and (diethoxy-phosphorylmethylsulfanyl)-acetic acid (0.13 g, 0.55 mmol) was coupled according to method B. The resin was washed with DCM (5×1 mL) and subsequently treated with a 20% 1,1,1,3,3,3-hexafluoro-propan-2-ol solution in DCM (4×3 mL) under agitation for 30 min in each case. The solvent of the combined filtrates was removed in vacuo to give the title compound as a yellow oil. MS (m/z): 354.3 [M+H⁺]

### B. {2-[2-(1-tert-butylcarbamoyl-2-naphthalen-2-yl-ethylcarbamoyl)-piperidin-1-yl]-2-oxo-ethylsulfanylmethyl}-phosphonic acid

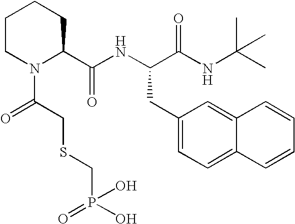

1-[2-(Diethoxy-phosphorylmethylsulfanyl)-acetyl]-piperidine-2-carboxylic acid (40 mg, 0.11 mmol) and 2-amino-N-tert-butyl-3-naphthalen-2-yl-propionamide (30 mg, 0.11 mmol) were coupled according to example 10B. The resulting crude phosphonic acid diethyl ester was deprotected according to method E. The residue was dissolved in a mixture of ACN (2 mL) and water (1 mL) and the pH of the solution was adjusted to 7 by the addition of solid NaHCO₃. After stirring for 20 h at room temperature under an argon atmosphere, the reaction mixture was concentrated in vacuo and purified by reversed phase HPLC to give the title compound as a white solid. MS (m/z): 550.5 [M+H⁺].

### EXAMPLE 12

### Synthesis of {2-[2-(1-carbamoyl-2-naphthalen-2-yl-ethylcarbamoyl)-piperidin-1-yl]-1-methylcarbamoylmethyl-2-oxo-ethylsulfanylmethyl}-phosphonic acid

### A. 1-[3-Carboxy-2-(diethoxy-phosphorylmethylsulfanyl)-propionyl]-piperidine-2-carboxylic acid methyl ester

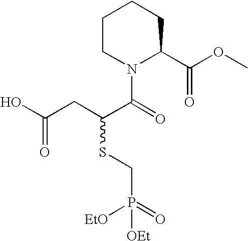

(5-Oxo-2,2-bis-trifluoromethyl-[1,3]oxathiolan-4-yl)-acetic acid (0.70 g, 2.3 mmol) was dissolved in anhydrous DMF (6 mL) and piperidine-2-carboxylic acid methyl ester hydrochloride (0.38 g, 2.1 mmol) was added to the stirred reaction mixture, followed by dropwise addition of DIEA (0.79 mL, 0.60 g, 4.7 mmol). After 80 min at room temperature, the solvent was removed in vacuo and the residue was dissolved in ethyl acetate (60 mL). The solution was extracted with aqueous HCl (2 M, 2×4 mL), dried over Na₂SO₄, filtered, and concentrated in vacuo. The residue was dissolved in a mixture of water (8 mL) and dioxane (3 mL). Iodomethyl-phosphonic acid diethyl ester (0.65 g, 2.3 mmol) and subsequently NaHCO₃ (0.59 g, 7.0 mmol) were added to the stirred reaction mixture. After stirring for 14 h at 55° C., the reaction mixture was concentrated in vacuo to a volume of approximately 10 mL. 1 M aqueous Na₂CO₃ (4 mL) was added and the aqueous solution was extracted with ethyl acetate (10 mL). The aqueous layer was then acidified with 6 M aqueous HCl to pH 1 and extracted with ethyl acetate (3×30 mL). The combined organic layers were dried over Na₂SO₄, filtered, and concentrated in vacuo. The residue was purified by flash chromatography over silica gel (elution with DCM/methanol/acetic acid 40:10:1) to give the title compound as a colorless oil. MS (m/z): 426.5 [M+H⁺].

### B. 1-[2-(Diethoxy-phosphorylmethylsulfanyl)-3-methylcarbamoyl-propionyl]-piperidine-2-carboxylic acid

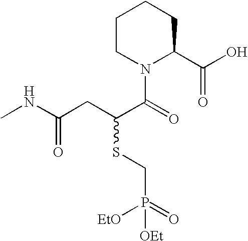

1-[3-Carboxy-2-(diethoxy-phosphorylmethylsulfanyl)-propionyl]-piperidine-2-carboxylic acid methyl ester (48 mg, 0.11 mmol) and methylamine (2 M in THF, 82 μL, 0.16 mmol) were coupled according to example 10B. The resulting N-methyl amide was dissolved in THF (1 mL) and a solution of LiOH (5.5 mg, 0.13 mmol) in water (0.20 mL) was added. After stirring for 4 h, the reaction mixture was concentrated in vacuo. The residue was partitioned between ethyl ethyl acetate (5 mL) and water (10 mL). The aqueous layer was acidified with 6 M aqueous HCl to pH 1, and extracted with ethyl acetate (4×7 mL). The combined organic layers were dried over Na₂SO₄, filtered, and concentrated in vacuo to give the crude title compound as a colorless oil. MS (m/z): 425.2 [M+H⁺].

### C. {2-[2-(1-Carbamoyl-2-naphthalen-2-yl-ethylcarbamoyl)-piperidin-1-yl]-1-methylcarbamoylmethyl-2-oxo-ethylsulfanylmethyl}-phosphonic acid

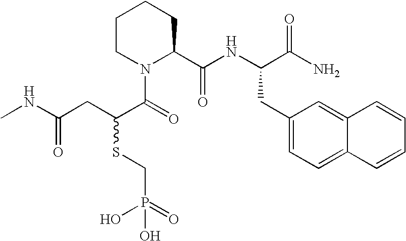

Crude 1-[2-(diethoxy-phosphorylmethylsulfanyl)-3-methylcarbamoyl-propionyl]-piperidine-2-carboxylic acid (36 mg, 84 μmol) and 2-amino-3-naphthalen-2-yl-propionamide (20 mg, 93 μmol) were coupled according to example 10B. The resulting crude phosphonic acid diethyl ester was deprotected according to method E and purified by reversed phase HPLC to give the title compound as a white solid. MS (m/z): 565.6 [M+H⁺].

### EXAMPLE 13

### Specificity of Inhibition of Certain Enzymes by Compounds According to the Present Invention

In order to characterize the specificity of various compounds the following assays were performed. PPIase activity of hPin1, hCyp18, LpCyp18, hFKBP12 and EcParvulin was measured using the protease-coupled PPIase assay according to Fischer et al. (Fischer, G.; Bang, H.; Mech, C. Determination of enzymatic catalysis for the cis-trans-isomerization of peptide binding in proline-containing peptides. [German] Biomed. Biochem. Acta 1984, 43, 1101-1111; Hennig et al., Selective Inactivation of Parvulin-like peptidyl-prolyl cis/trans isomerases by Juglon, Biochemistry. 1998, 37(17):5953-5960). For hPin1 measurements Ac-Ala-Ala-Ser(PO₃H₂)-Pro-Arg-pNA was used as a substrate and trypsin (final concentration 190 μg/ml) as an isomer-specific protease. Activity measurements of other PPIases were made with the substrate peptide Suc-Ala-Phe-Pro-Phe-pNA and the protease α-chymotrypsin (final concentration 470 μg/ml). The assays were performed in a final reaction volume of 150 μl at final concentrations of 6 nM hPin1, 10 μM hCyp18, 5 nM LpCyp18, 20 nM EcParvulin and 20 nM hFKBP12, respectively, and 120 μM substrate peptide in 35 mM HEPES (pH 7.8). For inhibition experiments 100-0.01 μM of effector freshly diluted from a DMSO stock solution were added. The amount of solvent was kept constant within each experiment, usually below 0.3% (v/v). All reactions were started by addition of protease. The test was performed by observing the released 4-nitroaniline at 390 nm with a MR5000 UV/Vis spectrophotometer (Dynex) at 6° C. Data were evaluated by calculation of pseudo-first-order rate constants k_{obs} in presence of PPIase and PPIase/effector, respectively, and corrected for the contribution of the non-catalyzed reaction (k₀). Inhibition constants IC₅₀ were calculated using SigmaPlot 8.0 (SPSS).

The following target enzymes which are all rotamases belonging to different classes of rotamases were used:

T-1: Protein interacting with NIMA (-kinase), hPin1
T-2: First described human Rapamycin receptor, hFKBP12
T-3: Human Cyclosporin A receptor with 18 kDa molecular weight, hCyp18
T-4: Leishmonia pneumophila virulence Cyclosporin A receptor with 18 kDa molecular weight, LpCyp18
T-5: Bacterial Juglon sensitive non proteolytic enzyme, EcParv

These rotamases are known in the art. Their production and characteristics may be taken from the following references.

### Review About All PPIase Families

Gothel, S. F.; Marahiel, M. A. TI Peptidyl-prolyl cis-trans isomerases, a superfamily of ubiquitous folding catalysts [Review]. Cell. Molec. Life Sci. 1999, 55, 423-436

### Pin1

Lu, K. P.; Hanes, S. D.; Hunter, T. (1996) A human peptidyl-prolyl isomerase essential for regulation of mitosis. Nature 1996, 380, 544-547
Yaffe, M. B.; Schutkowski, M.; Shen, M. H.; Zhou, X. Z.; Stukenberg, P. T.; Rahfeld, J. U.; Xu, J.; Kuang, J.; Kirschner, M. W.; Fischer, G.; Cantley, L. C.; Lu K. P. Sequence-specific and phosphorylation dependent praline isomerisation—A potential mitotic regulatory mechanism. Science 1997, 278, 1957-1960
Shen, M.; Stukenberg, P. T.; Kirschner, M. W.; Lu, K. P. The essential mitotic peptidyl-prolyl isomerase Pin1 binds and regulates mitosis-specific phosphoproteins. Genes Developm. 1998, 12, 706-720.

### EcParvulin

Rahfeld J U. Schierhorn A. Mann K. Fischer G. A novel peptidyl-prolyl cis/trans isomerase from Escherichia coli. FEBS Letters. 1994, 343, 65-69
Rahfeld J U. Rucknagel K P. Schelbert B. Ludwig B. Hacker J. Mann K. Fischer G. Confirmation of the existence of a third family among peptidyl-prolyl cis/trans isomerases. Amino acid sequence and recombinant production of parvulin. FEBS Letters. 1994, 352, 180-184
   FKBPs (including FKBP12) and Cyclophilins (including Cyp18)

For recent reviews on cyclophilins and FKBPs and their effectors, see: (a) Fischer, G. Peptidyl-prolyl cis/trans isomerases and their effectors. Angew. Chem., Int. Ed. Engl. 1994, 33, 1415-1436. (b) Galat, A.; Metcalfe, S. M. Peptidylproline cis/trans isomerases. Prog. Biophys. Molec. Biol. 1995, 63, 67-118.

### LpCyp18

Schmidt B. Tradler T. Rahfeld J U. Ludwig B. Jain B. Mann K. Rucknagel K P. Janowski B. Schierhorn A. Kullertz G. Hacker J. Fischer G. A cyclophilin-like peptidyl-prolyl cis/trans isomerase from Legionella pneumophila—characterization, molecular cloning and overexpression. Mol. Microbiol. 1996, 21, 1147-1160

In order to cluster the various rotamase inhibitors the following classes were defined with “A” indicating the most potent rotamase inhibitor.

TABLE 2
Specificity of the inhibition with rotamases

| | | Target | | | | |
|---|---|---|---|---|---|---|
| Compound | N° | T-1 | T-2 | T-3 | T-4 | T-5 |
| 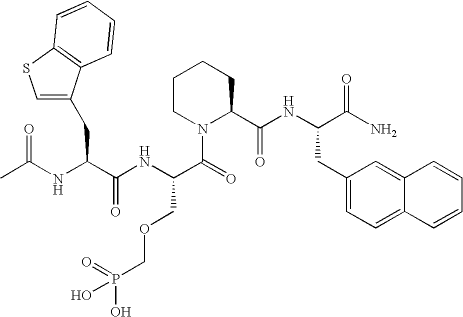 | 1 | B | — | — | — | — |
| 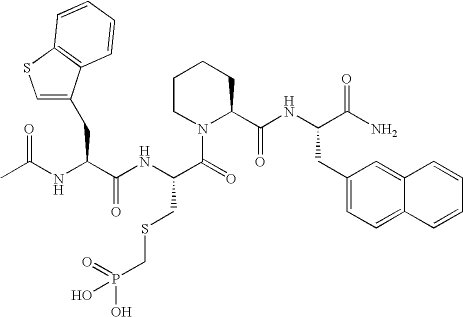 | 2 | A | — | — | — | — |
| 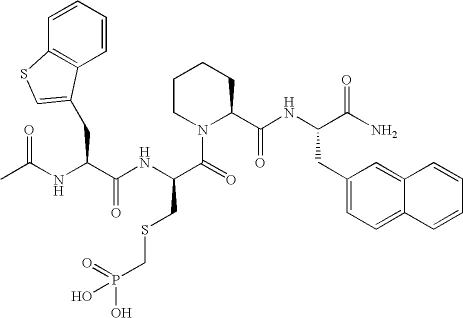 | 3 | A | — | — | — | — |
| 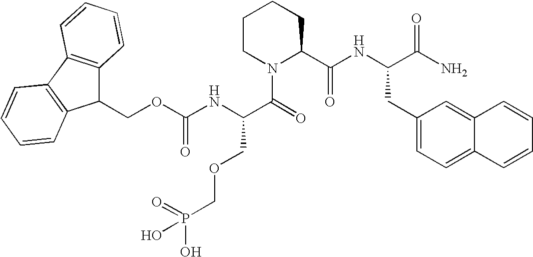 | 4 | B | — | — | — | — |
| 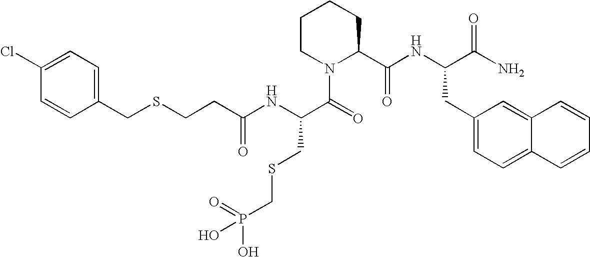 | 5 | A | — | — | — | — |
| 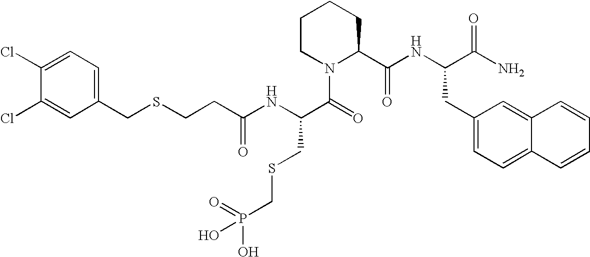 | 6 | B | — | — | — | — |
| 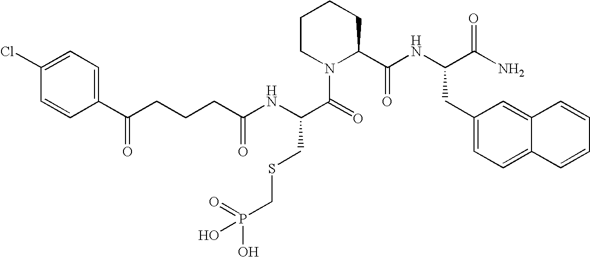 | 7 | A | — | — | — | — |
| 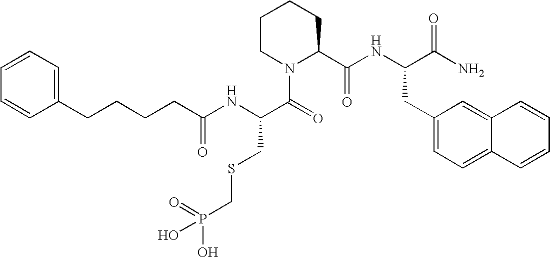 | 8 | A | — | — | — | — |
| 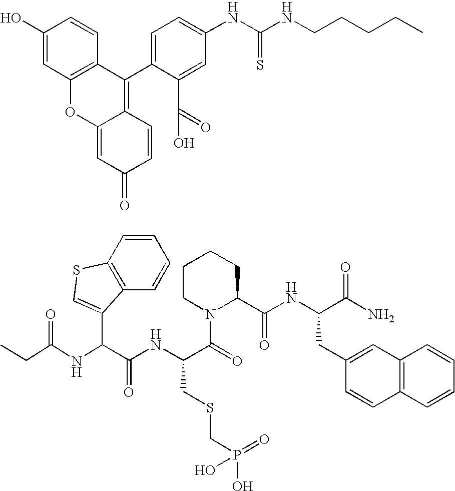 | 9 | A | — | — | — | — |
| 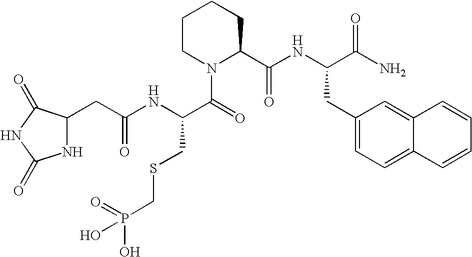 | 10 | A | — | — | — | — |
| 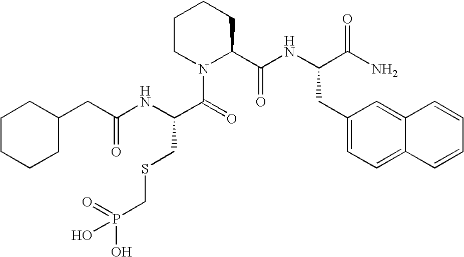 | 11 | B | — | — | — | — |
| 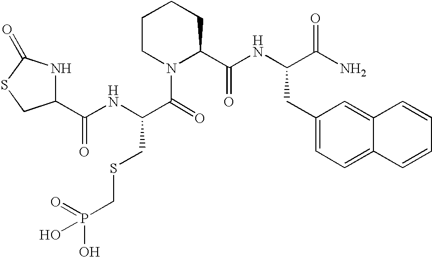 | 12 | A | — | — | — | — |
| 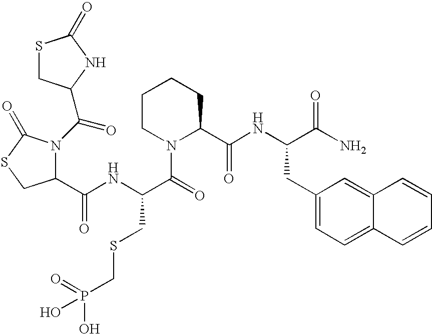 | 13 | A | — | — | — | — |
| 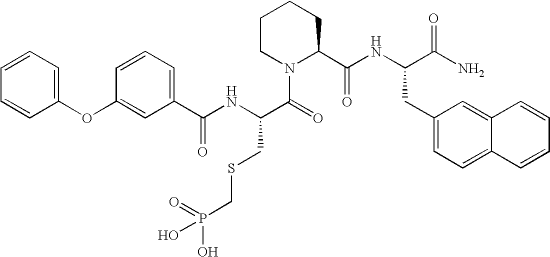 | 14 | A | — | — | — | — |
| 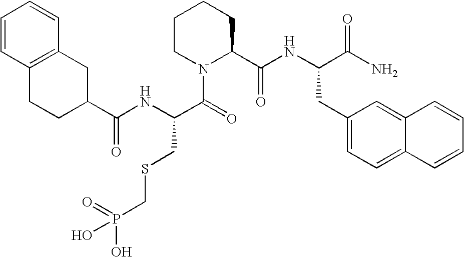 | 15 | B | — | — | — | — |
| 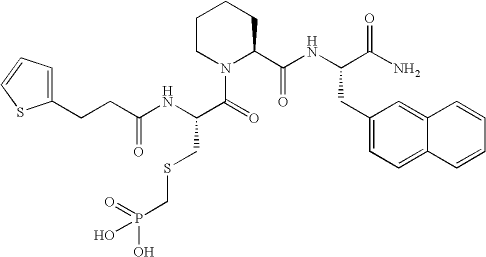 | 16 | B | — | — | — | — |
| 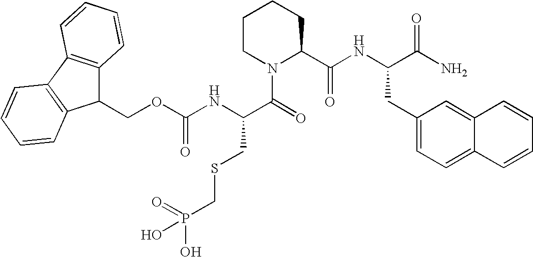 | 17 | B | — | — | — | — |
| 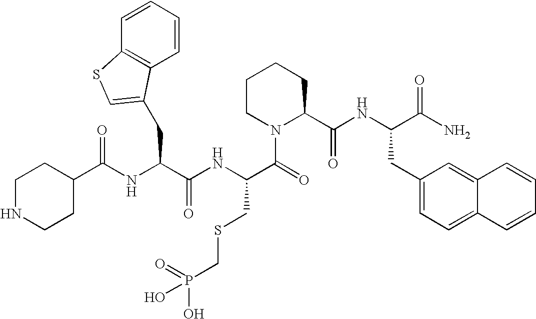 | 18 | A | — | — | — | — |
| 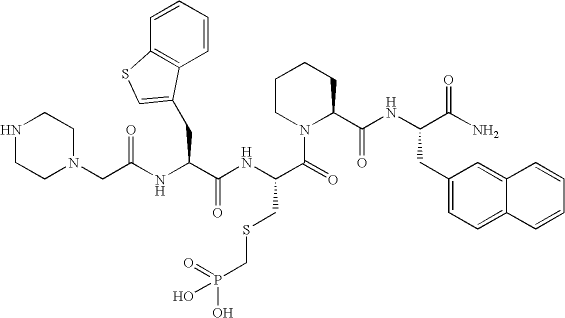 | 19 | A | — | — | — | — |
| 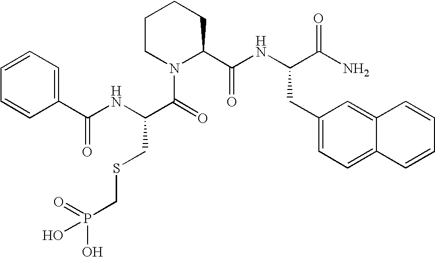 | 20 | A | — | — | — | — |
| 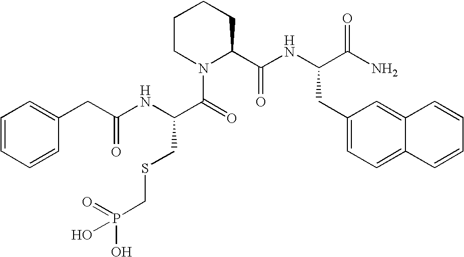 | 21 | A | — | — | — | — |
| 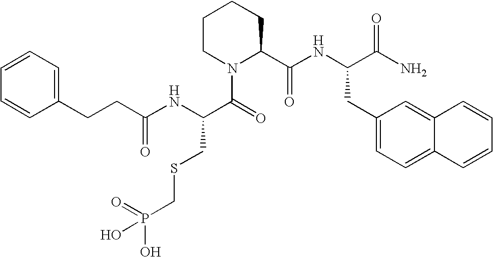 | 22 | A | — | — | — | — |
| 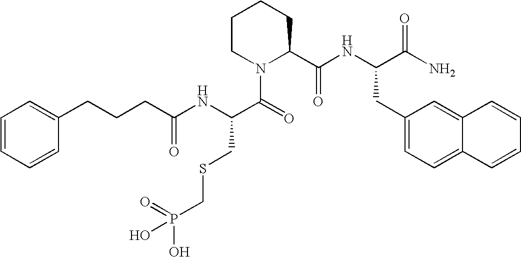 | 23 | A | — | — | — | — |
| 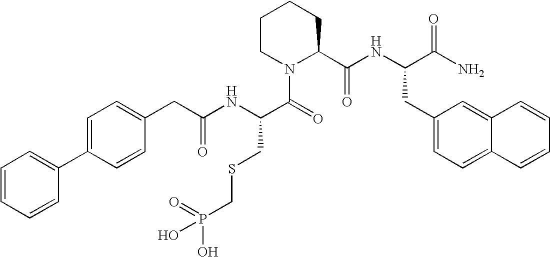 | 24 | A | — | — | — | — |
| 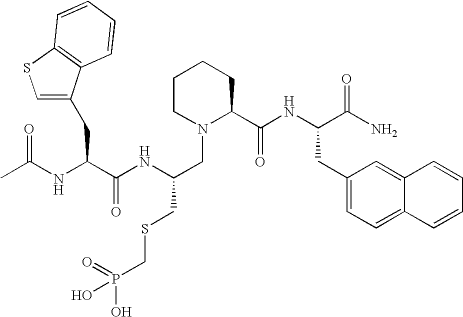 | 25 | A | — | — | — | — |
| 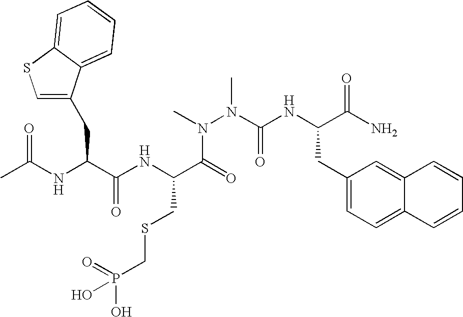 | 26 | A | — | — | — | — |
| 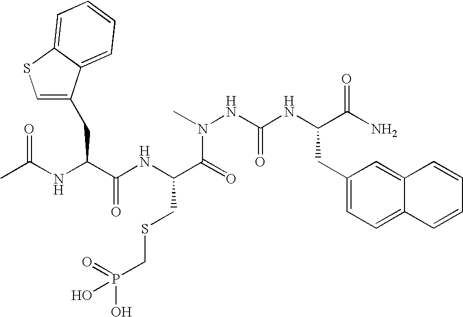 | 27 | A | — | — | — | — |
| 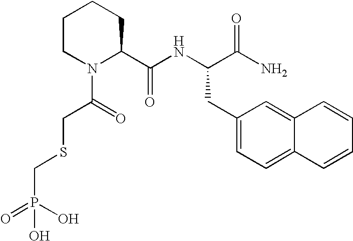 | 28 | A | — | — | — | — |
| 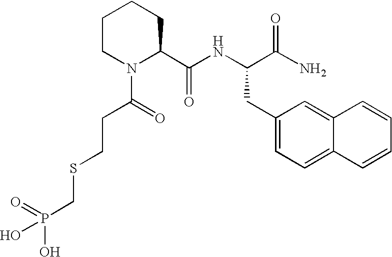 | 29 | A | — | — | — | — |
| 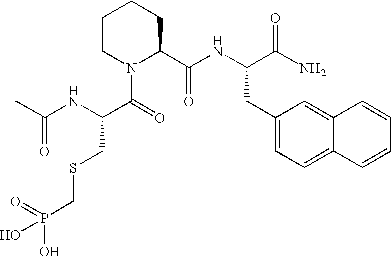 | 30 | B | — | — | — | — |
| 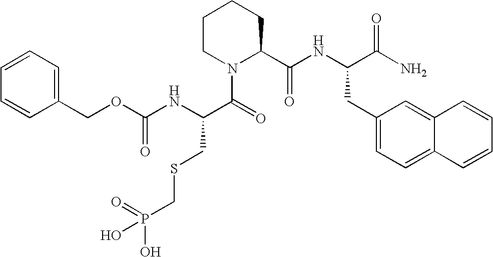 | 31 | A | — | — | — | — |
| 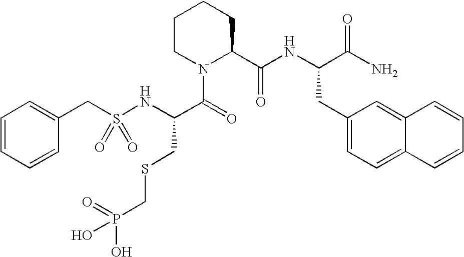 | 32 | A | — | — | — | — |
| 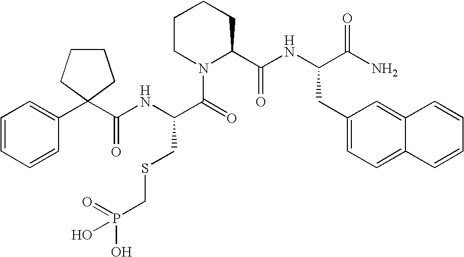 | 33 | B | — | — | — | — |
| 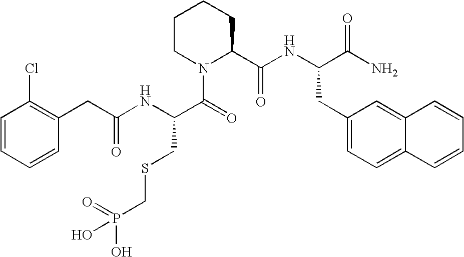 | 34 | A | — | — | — | — |
| 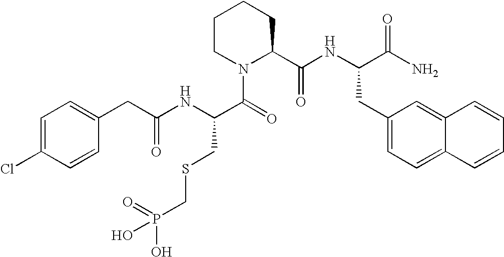 | 35 | A | — | — | — | — |
| 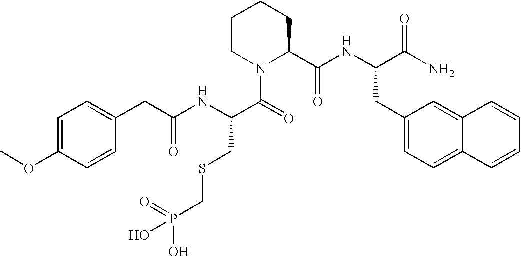 | 36 | A | — | — | — | — |
| 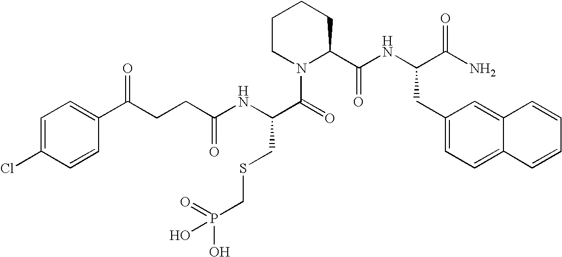 | 37 | A | — | — | — | — |
| 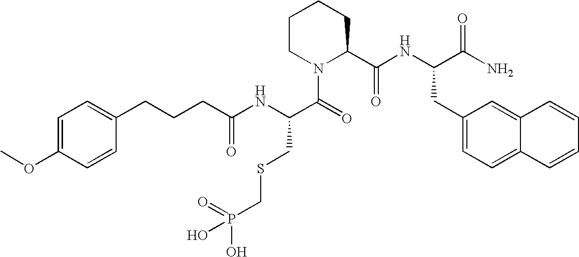 | 38 | A | — | — | — | — |
| 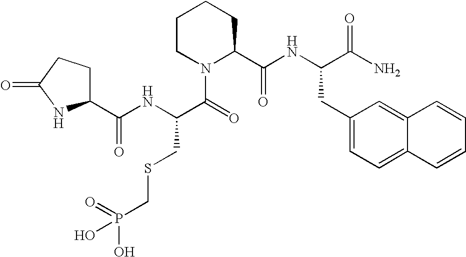 | 39 | A | — | — | — | — |
| 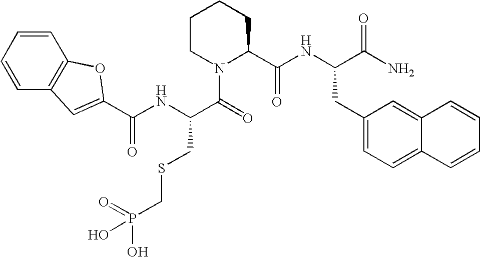 | 40 | B | — | — | — | — |
| 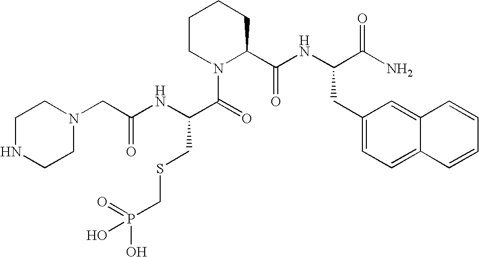 | 41 | B | — | — | — | — |
| 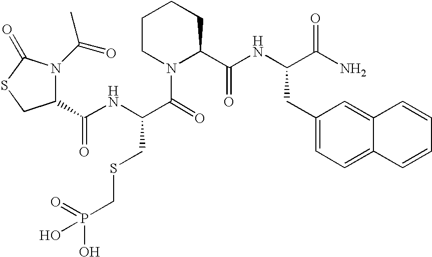 | 42 | A | — | — | — | — |
| 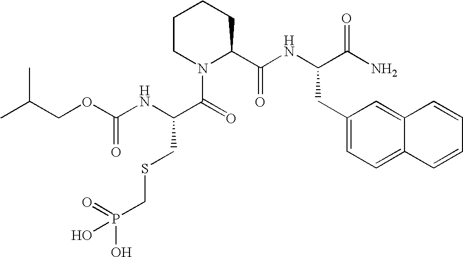 | 43 | B | — | — | — | — |
| 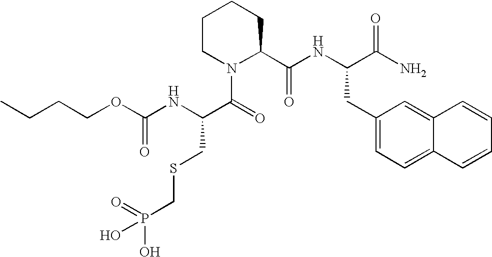 | 44 | B | — | — | — | — |
| 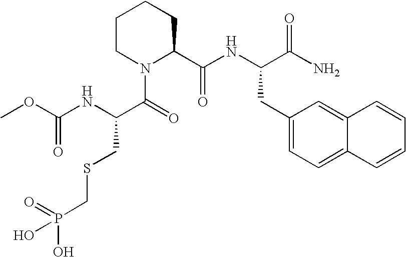 | 45 | B | — | — | — | — |
| 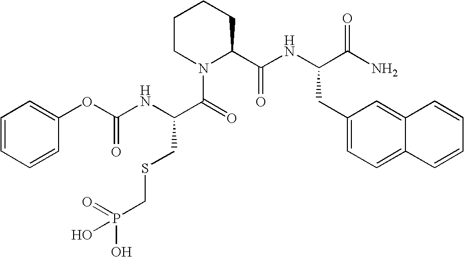 | 46 | A | — | — | — | — |
| 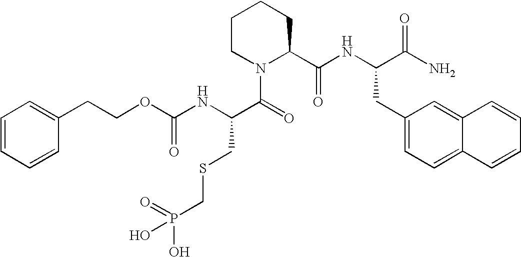 | 47 | B | — | — | — | — |
| 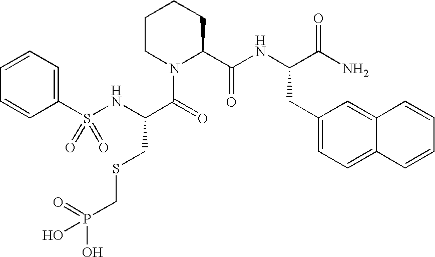 | 48 | A | — | — | — | — |
| 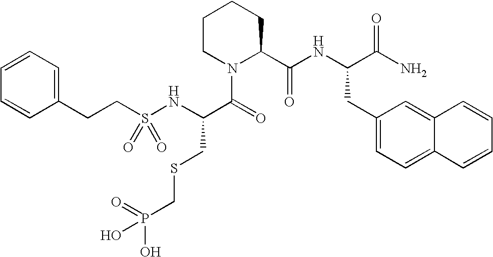 | 49 | A | — | — | — | — |
| 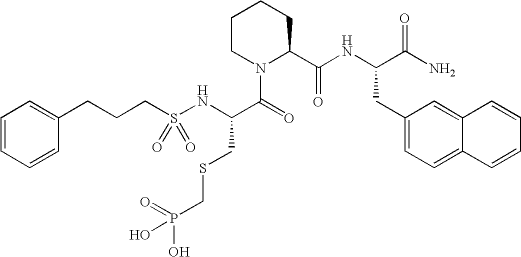 | 50 | A | — | — | — | — |
| 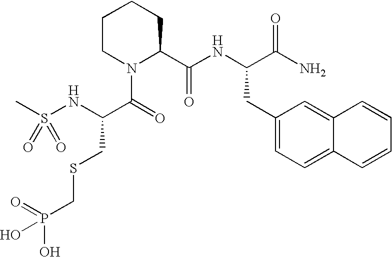 | 51 | B | — | — | — | — |
| 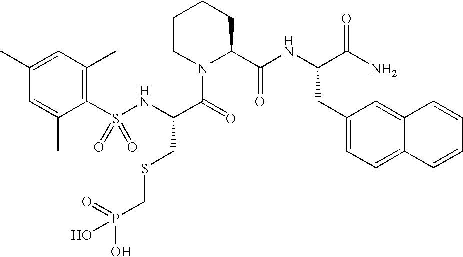 | 52 | B | — | — | — | — |
| 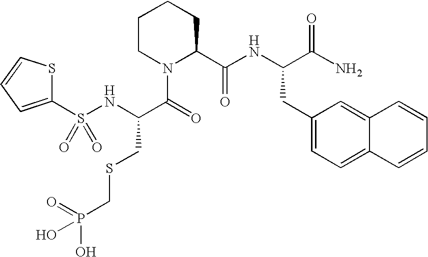 | 53 | A | — | — | — | — |
| 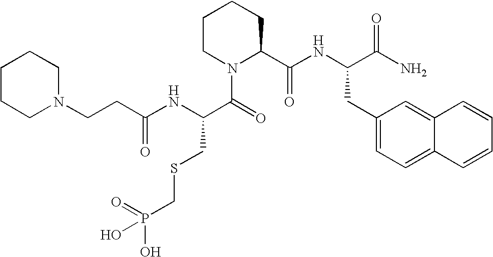 | 54 | B | — | — | — | — |
| 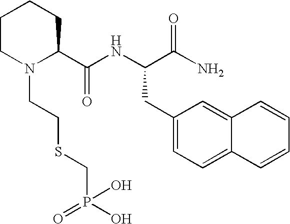 | 55 | A | — | — | — | — |
| 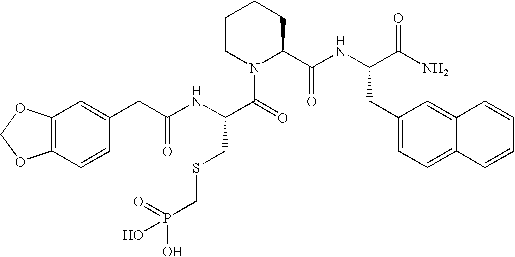 | 56 | A | — | — | — | — |
| 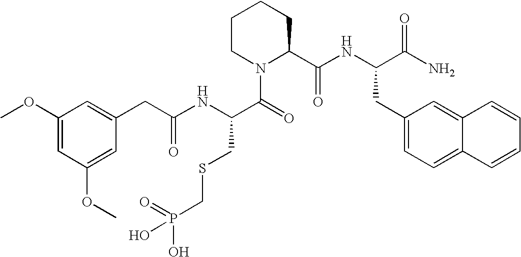 | 57 | A | — | — | — | — |
| 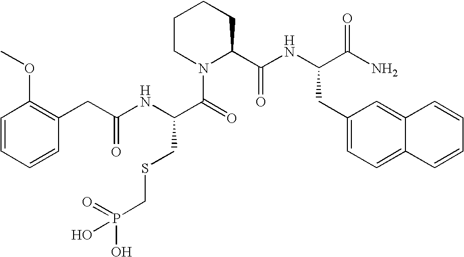 | 58 | A | — | — | — | — |
| 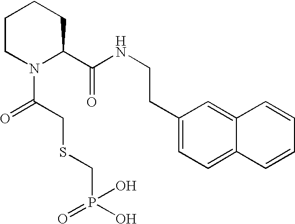 | 59 | B | — | — | — | — |
| 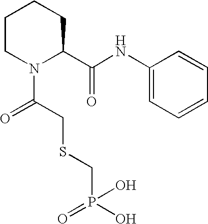 | 60 | B | — | — | — | — |
| 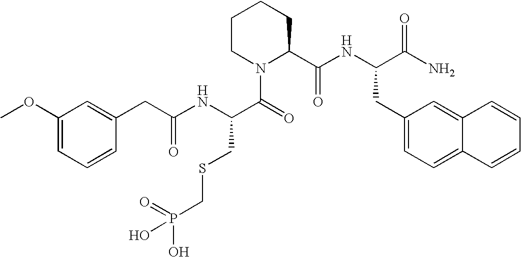 | 61 | A | — | — | — | — |
| 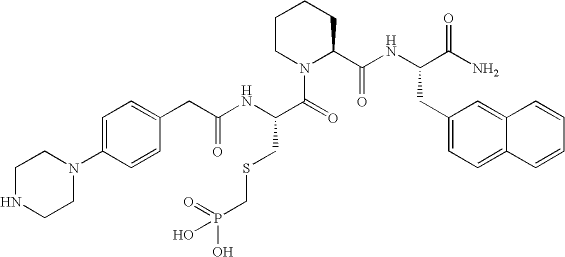 | 62 | A | — | — | — | — |
| 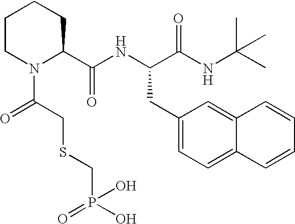 | 63 | A | — | — | — | — |
| 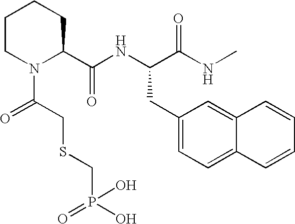 | 64 | A | — | — | — | — |
| 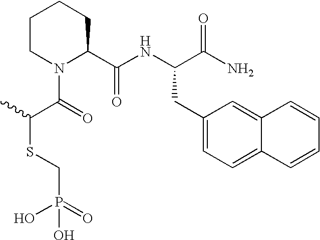 | 65 | B | — | — | — | — |
| 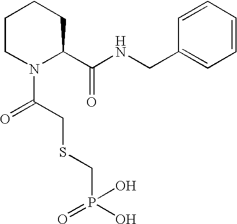 | 66 | B | — | — | — | — |
| 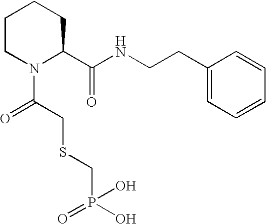 | 67 | B | — | — | — | — |
| 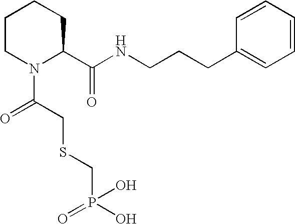 | 68 | B | — | — | — | — |
| 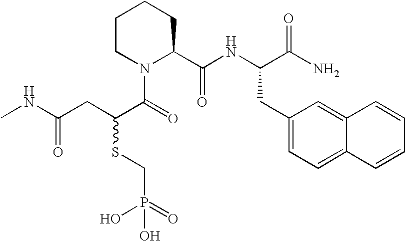 | 69 | B | — | — | — | — |
| 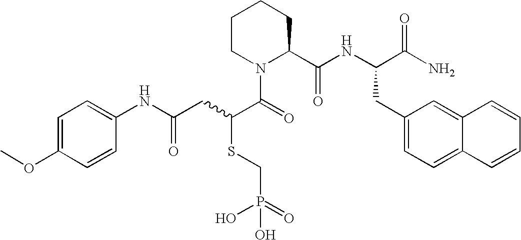 | 70 | A | — | — | — | — |
| 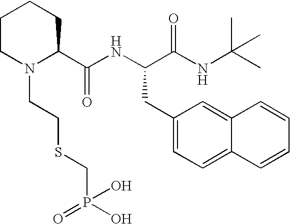 | 71 | A | — | — | — | — |
| 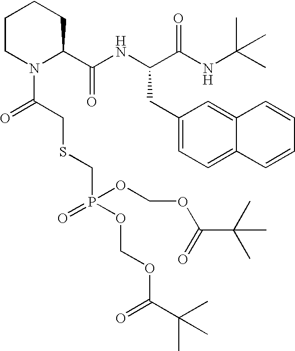 | 72 | — | — | — | — | — |
| 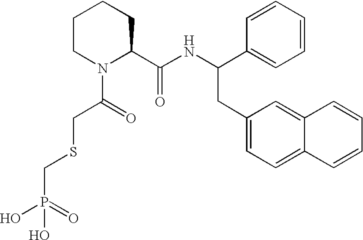 | 73 | A | — | — | — | — |
| 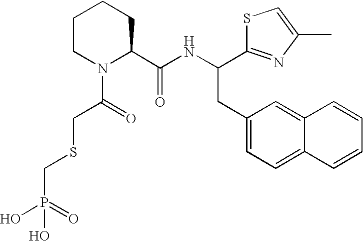 | 74 | B | — | — | — | — |
| 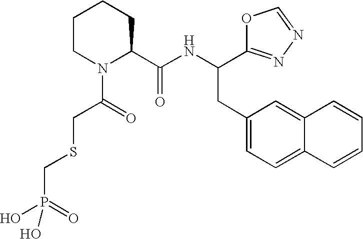 | 75 | A | — | — | — | — |
| 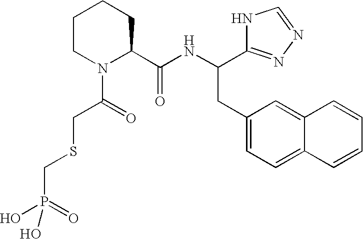 | 76 | A | — | — | — | — |
| 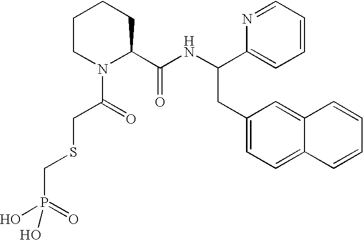 | 77 | A | — | — | — | — |

A: IC₅₀ < 1 μM
B: 1 μM < IC₅₀ < 10 μM
C: 10 μM < IC₅₀ < 50 μM
D: 50 μM < IC₅₀ < 100 μM
E: IC₅₀ > 100 μM

The features of the present invention disclosed in the specification, the claims and/or the drawings may both separately and in any combination thereof be material for realizing the invention in various forms thereof.

1. A compound having formula (I) 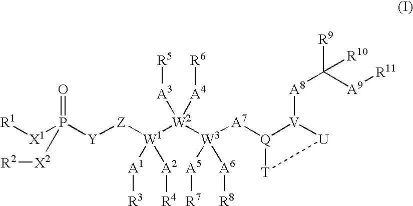
wherein the dashed line indicates a single or double bond, or is absent;
wherein R¹ and R² are each and independently selected from the group comprising —H and phospho protecting groups;
wherein X¹ and X² are each and independently selected from the group comprising —O—, —S—, —NR¹²—;
wherein Z is selected from the group comprising —O—, —S—, —NR¹³—, —(CR¹⁴R¹⁵)—;
wherein A¹, A², A³, A⁴, A⁵, A⁶, A⁷, A⁸ and A⁹ are each and independently selected from the group comprising —O—, —S—, —NR¹⁶—, —S(O)—, —S(O₂)—, —C(O)—, —C(S)—, —NR¹⁷—C(O)—, —NR¹⁸—C(S)—, —NR¹⁹—C(O)—NR²⁰—, —NR²¹C(S)—NR²²—, —NR²³—S(O)—, —NR²⁴—S(O₂)—, and —NR²⁵—C(O)—O—, or are each and independently from each other absent;
wherein Y is selected from the group comprising —O—, —CR²⁶R²⁷—;
wherein Q and V are each and independently selected from the group comprising 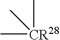
and 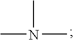
wherein W¹, W² and W³ are each and independently selected from the group comprising 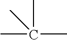
and 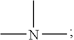
wherein R³, R⁴, R⁵, R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, R²¹, R²², R²³, R²⁴, R²⁵, R²⁶, R²⁷, R²⁸, T and U are each and independently selected from the group comprising —H, halo, alkyl, substituted alkyl, straight alkyl, substituted straight alkyl, branched alkyl, substituted branched alkyl, alkenyl, straight alkenyl, substituted straight alkenyl, branched alkenyl, substituted branched alkenyl, alkynyl, straight alkynyl, substituted straight alkynyl, branched alkynyl, substituted branched alkynyl, cycloalkyl, substituted cycloalkyl, cycloalkenyl, substituted cycloalkenyl, heterocyclyl, substituted heterocyclyl, mono-unsaturated heterocyclyl, substituted mono-unsaturated heterocyclyl, poly-unsaturated heterocyclyl, substituted poly-unsaturated heterocyclyl, aryl, substituted aryl, heteroaryl, substituted heteroaryl, arylalkyl, substituted arylalkyl, heteroarylalkyl, substituted heteroarylalkyl, heterocyclylalkyl, substituted and heterocyclylalkyl, or are each and independently from each other absent;
and the salts, hydrates, solvates and prodrugs thereof.

2. The compound according to claim 1, wherein W¹, A¹, A², A³, A⁴, A⁵, R³, and R⁴ are absent; wherein R⁵, R⁶ and R⁷ are —H; wherein W² and W³ is 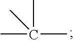
wherein preferably Z is either —S— or —O—, more preferably —S—; and wherein preferably Y is —CH₂—; wherein preferably A⁷ is either —C(O)— or —CH₂—; wherein both X¹ and X² are —O—; wherein A⁸ is —C(O)—O— or —NR²⁹—C(O)—, whereby the C-atom of the —NR²⁹—C(O)— and —C(O)—O— is covalently bound to V; and
wherein R²⁹ is —H or lower alkyl.

3. The compound according to claim 1, wherein W¹, W², A¹, A², A³, A⁴, A⁵, R³, R⁴, R⁵, and R⁶ are absent; wherein R⁷ is —H; wherein W³ is 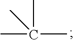
wherein preferably Z is either —S— or —O—, more preferably —S—; and wherein preferably Y is —CH₂—; wherein preferably A⁷ is either —C(O)— or —CH₂—; wherein both X¹ and X² are —O—; wherein A⁸ is —C(O)—O— or —NR²⁹—C(O)—, whereby the C-atom of the —NR²⁹—C(O)— and —C(O)—O— is covalently bound to V; and wherein R²⁹ is —H or lower alkyl.

4. The compound according to claim 3, wherein R⁸ is —H and wherein preferably A⁶ is absent.

5. The compound according to claim 3, wherein A⁶ is selected from the group comprising —NR¹⁷—C(O)—, —NR²⁴—S(O₂)—, —NR²⁵—C(O)—O—, and wherein R⁸ is selected from the group comprising optionally substituted aryl-(lower alkyl), optionally substituted heteroaryl-(lower alkyl), optionally substituted aryl and optionally substituted heteroaryl, preferably optionally substituted phenyl, optionally substituted phenyl-(lower alkyl) and more preferably 1-acetylamino-2-benzo[b]thiophen-3-yl-ethyl; dihalo-benzylsulfanylethyl, monohalo-benzylsulfanylethyl, -4-(monohalo-phenyl)-4-oxo-butyl, 4-(dihalo-phenyl)-4-oxo-butyl; benzo[1,3]dioxol-5-ylmethyl, wherein R¹⁷, R²⁴ and R²⁵ are each and independently selected from the group comprising —H and lower alkyl.

6. The compound according to claim 4, wherein Q and V are 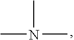
wherein T and U are alkyl, preferably lower alkyl, and wherein the dashed line is absent or a single bond.

7. The compound according to claim 4, wherein R²⁸ is —H or lower alkyl, wherein the dashed line is a single bond, wherein T is —CH₂— and wherein U is selected from the group comprising —(CH₂)ₙ—; wherein n is any integer from 1 to and preferably 2, 3 or 4.

8. The compound according to claim 4, wherein R⁹ is —H and wherein R¹⁰ is selected from the group comprising substituted lower alkyl, preferably aryl-(lower-alkyl), heteroaryl-(lower-alkyl), cycloalkyl-(lower-alkyl), heterocyclyl-(lower-alkyl), and more preferably optionally substituted 2-naphthalen-2-yl-ethyl, optionally substituted naphthalen-2-ylmethyl, optionally substituted 2-phenyl-ethyl, optionally substituted 2-phenyl-methyl, optionally substituted quinolin-7-ylmethyl-, and optionally substituted 3-isoquinolin-7-ylmethyl.

9. The compound according to claim 4 wherein A⁹ is —NH—C(O)— or NH—C(S)—, whereby the N-atom of each of —NH—C(O)— and NH—C(S)— is covalently bound to R¹¹, and wherein R¹¹ is selected from the group comprising —H, optionally substituted alkyl, optionally substituted aryl, optionally substituted heteroaryl, optionally substituted heterocyclyl, preferably optionally substituted lower alkyl or —H, and more preferably optionally substituted tert-butyl or optionally substituted isopropyl.

10. The compound according to claim 4, wherein A⁹ is absent and wherein R¹¹ is selected from the group comprising optionally substituted alkyl, optionally substituted aryl, optionally substituted heteroaryl, optionally substituted heterocyclyl, preferably optionally substituted phenyl, optionally substituted thiazol-2-yl, optionally substituted pyridyl and optionally substituted [1,3,4]oxadiazol-2-yl, optionally substituted 4H-[1,2,4]triazol-3-yl.

11. The compound according to claim 4 wherein both R¹ and R² are —H.

12. The compound according to claim 4, wherein R¹ and R² are each a phospho protecting group, whereby preferably R¹ and R² are each and independently selected from the group comprising 2,2-dimethyl-propionyloxymethyl, isopropoxycarbonyloxymethyl, and 2-acetylsulfanyl-ethyl.

13. A compound, preferably a compound according to claim 1, selected from the group comprising
{2-(2-Acetylamino-3-benzo[b]thiophen-3-yl-propionylamino)-3-[2-(1-carbamoyl-2-naphthalen-2-yl-ethylcarbamoyl)-piperidin-1-yl]-3-oxo-propoxymethyl}-phosphonic acid
{2-(2-Acetylamino-3-benzo[b]thiophen-3-yl-propionylamino)-3-[2-(1-carbamoyl-2-naphthalen-2-yl-ethylcarbamoyl)-piperidin-1-yl]-3-oxo-propylsulfanylmethyl}-phosphonic acid
{2-(2-Acetylamino-3-benzo[b]thiophen-3-yl-propionylamino)-3-[2-(1-carbamoyl-2-naphthalen-2-yl-ethylcarbamoyl)-piperidin-1-yl]-3-oxo-propylsulfanylmethyl}-phosphonic acid
[3-[2-(1-Carbamoyl-2-naphthalen-2-yl-ethylcarbamoyl)-piperidin-1-yl]-2-(9H-fluoren-9-ylmethoxycarbonylamino)-3-oxo-propoxymethyl]-phosphonic acid
{3-[2-(1-Carbamoyl-2-naphthalen-2-yl-ethylcarbamoyl)-piperidin-1-yl]-2-[3-(4-chloro-benzylsulfanyl)-propionylamino]-3-oxo-propylsulfanylmethyl}-phosphonic acid
{3-[2-(1-Carbamoyl-2-naphthalen-2-yl-ethylcarbamoyl)-piperidin-1-yl]-2-[3-(3.4-dichloro-benzylsulfanyl)-propionylamino]-3-oxo-propylsulfanylmethyl}-phosphonic acid
{3-[2-(1-Carbamoyl-2-naphthalen-2-yl-ethylcarbamoyl)-piperidin-1-yl]-2-[5-(4-chloro-phenyl)-5-oxo-pentanoylamino]-3-oxo-propylsulfanylmethyl}-phosphonic acid
[3-[2-(1-Carbamoyl-2-naphthalen-2-yl-ethylcarbamoyl)-piperidin-1-yl]-3-oxo-2-(5-phenyl-pentanoylamino)-propylsulfanylmethyl]-phosphonic acid
{2-(3-Benzo[b]thiophen-3-yl-2-{6-[5-(-2-(6-hydroxy-3-oxo-3H-xanthen-9-yl)-benzoic acid)-ureido]-hexanoylamino}-propionylamino)-3-[2-(1-carbamoyl-2-naphthalen-2-yl-ethylcarbamoyl)-piperidin-1-yl]-3-oxo-propylsulfanylmethyl}-phosphonic acid
{3-[2-(1-Carbamoyl-2-naphthalen-2-yl-ethylcarbamoyl)-piperidin-1-yl]-2-[2-(2.5-dioxo-imidazolidin-4-yl)-acetylamino]-3-oxo-propylsulfanylmethyl}-phosphonic acid
[3-[2-(1-Carbamoyl-2-naphthalen-2-yl-ethylcarbamoyl)-piperidin-1-yl]-2-(2-cyclohexyl-acetylamino)-3-oxo-propylsulfanylmethyl]-phosphonic acid
{3-[2-(1-Carbamoyl-2-naphthalen-2-yl-ethylcarbamoyl)-piperidin-1-yl]-3-oxo-2-[(2-oxo-thiazolidine-4-carbonyl)-amino]-propylsulfanylmethyl}-phosphonic acid
(3-[2-(1-Carbamoyl-2-naphthalen-2-yl-ethylcarbamoyl)-piperidin-1-yl]-3-oxo-2-{[2-oxo-3-(2-oxo-thiazolidine-4-carbonyl)-thiazolidine-4-carbonyl]-amino-}-propylsulfanylmethyl)-phosphonic acid
[3-[2-(1-Carbamoyl-2-naphthalen-2-yl-ethylcarbamoyl)-piperidin-1-yl]-3-oxo-2-(3-phenoxy-benzoylamino)-propylsulfanylmethyl]-phosphonic acid
{3-[2-(1-Carbamoyl-2-naphthalen-2-yl-ethylcarbamoyl)-piperidin-1-yl]-3-oxo-2-[(1.2.3.4-tetrahydro-naphthalene-2-carbonyl)-amino]-propylsulfanylmethyl}-phosphonic acid
[3-[2-(1-Carbamoyl-2-naphthalen-2-yl-ethylcarbamoyl)-piperidin-1-yl]-3-oxo-2-(3-thiophen-2-yl-propionylamino)-propylsulfanylmethyl]-phosphonic acid
[3-[2-(1-Carbamoyl-2-naphthalen-2-yl-ethylcarbamoyl)-piperidin-1-yl]-2-(9H-fluoren-9-ylmethoxycarbonylamino)-3-oxo-propylsulfanylmethyl]-phosphonic acid
{2-{3-Benzo[b]thiophen-3-yl-2-[(piperidine-4-carbonyl)-amino]-propionylamino}-3-[2-(1-carbamoyl-2-naphthalen-2-yl-ethylcarbamoyl)-piperidin-1-yl]-3-oxo-propylsulfanylmethyl}-phosphonic acid
{2-[3-Benzo[b]thiophen-3-yl-2-(2-piperazin-1-yl-acetylamino)-propionylamino]-3-[2-(1-carbamoyl-2-naphthalen-2-yl-ethylcarbamoyl)-piperidin-1-yl]-3-oxo-propylsulfanylmethyl}-phosphonic acid
{2-Benzoylamino-3-[2-(1-carbamoyl-2-naphthalen-2-yl-ethylcarbamoyl)-piperidin-1-yl]-3-oxo-propylsulfanylmethyl}-phosphonic acid
{3-[2-(1-Carbamoyl-2-naphthalen-2-yl-ethylcarbamoyl)-piperidin-1-yl]-3-oxo-2-phenylacetylamino-propylsulfanylmethyl}-phosphonic acid
[3-[2-(1-Carbamoyl-2-naphthalen-2-yl-ethylcarbamoyl)-piperidin-1-yl]-3-oxo-2-(3-phenyl-propionylamino)-propylsulfanylmethyl]-phosphonic acid
[3-[2-(1-Carbamoyl-2-naphthalen-2-yl-ethylcarbamoyl)-piperidin-1-yl]-3-oxo-2-(4-phenyl-butyrylamino)-propylsulfanylmethyl]-phosphonic acid
{2-(2-Biphenyl-4-yl-acetylamino)-3-[2-(1-carbamoyl-2-naphthalen-2-yl-ethylcarbamoyl)-piperidin-1-yl]-3-oxo-propylsulfanylmethyl}-phosphonic acid
{2-(2-Acetylamino-3-benzo[b]thiophen-3-yl-propionylamino)-3-[2-(1-carbamoyl-2-naphthalen-2-yl-ethylcarbamoyl)-piperidin-1-yl]-propylsulfanylmethyl}-phosphonic acid
Ac-Bta-Cys(CH₂—P(O)(OH)₂)—NMeazaAla-2Nal-NH₂
Ac-Bta-Cys(CH₂—P(O)(OH)₂)—NMeazaGly-2Nal-NH₂
{2-[2-(1-Carbamoyl-2-naphthalen-2-yl-ethylcarbamoyl)-piperidin-1-yl]-2-oxo-ethylsulfanylmethyl}-phosphonic acid
{3-[2-(1-Carbamoyl-2-naphthalen-2-yl-ethylcarbamoyl)-piperidin-1-yl]-3-oxo-propylsulfanylmethyl}-phosphonic acid
{2-Acetylamino-3-[2-(1-carbamoyl-2-naphthalen-2-yl-ethylcarbamoyl)-piperidin-1-yl]-3-oxo-propylsulfanylmethyl}-phosphonic acid
{2-Benzyloxycarbonylamino-3-[2-(1-carbamoyl-2-naphthalen-2-yl-ethylcarbamoyl)-piperidin-1-yl]-3-oxo-propylsulfanylmethyl}-phosphonic acid
{3-[2-(1-Carbamoyl-2-naphthalen-2-yl-ethylcarbamoyl)-piperidin-1-yl]-3-oxo-2-phenylmethanesulfonylamino-propylsulfanylmethyl}-phosphonic acid
{3-[2-(1-Carbamoyl-2-naphthalen-2-yl-ethylcarbamoyl)-piperidin-1-yl]-3-oxo-2-[(1-phenyl-cyclopentanecarbonyl)-amino]-propylsulfanylmethyl}-phosphonic acid
{3-[2-(1-Carbamoyl-2-naphthalen-2-yl-ethylcarbamoyl)-piperidin-1-yl]-2-[2-(2-chloro-phenyl)-acetylamino]-3-oxo-propylsulfanylmethyl}-phosphonic acid
{3-[2-(1-Carbamoyl-2-naphthalen-2-yl-ethylcarbamoyl)-piperidin-1-yl]-2-[2-(4-chloro-phenyl)-acetylamino]-3-oxo-propylsulfanylmethyl}-phosphonic acid
{3-[2-(1-Carbamoyl-2-naphthalen-2-yl-ethylcarbamoyl)-piperidin-1-yl]-2-[2-(4-methoxy-phenyl)-acetylamino]-3-oxo-propylsulfanylmethyl}-phosphonic acid
{3-[2-(1-Carbamoyl-2-naphthalen-2-yl-ethylcarbamoyl)-piperidin-1-yl]-2-[4-(4-chloro-phenyl)-4-oxo-butyrylamino]-3-oxo-propylsulfanylmethyl}-phosphonic acid
{3-[2-(1-Carbamoyl-2-naphthalen-2-yl-ethylcarbamoyl)-piperidin-1-yl]-2-[4-(4-methoxy-phenyl)-butyrylamino]-3-oxo-propylsulfanylmethyl}-phosphonic acid
{3-[2-(1-Carbamoyl-2-naphthalen-2-yl-ethylcarbamoyl)-piperidin-1-yl]-3-oxo-2-[(5-oxo-pyrrolidine-2-carbonyl)-amino]-propylsulfanylmethyl}-phosphonic acid
{2-[(Benzofuran-2-carbonyl)-amino]-3-[2-(1-carbamoyl-2-naphthalen-2-yl-ethylcarbamoyl)-piperidin-1-yl]-3-oxo-propylsulfanylmethyl}-phosphonic acid
[3-[2-(1-Carbamoyl-2-naphthalen-2-yl-ethylcarbamoyl)-piperidin-1-yl]-3-oxo-2-(2-piperazin-1-yl-acetylamino)-propylsulfanylmethyl]-phosphonic acid
{2-[(3-Acetyl-2-oxo-thiazolidine-4-carbonyl)-amino]-3-[2-(1-carbamoyl-2-naphthalen-2-yl-ethylcarbamoyl)-piperidin-1-yl]-3-oxo-propylsulfanylmethyl}-phosphonic acid
{3-[2-(1-Carbamoyl-2-naphthalen-2-yl-ethylcarbamoyl)-piperidin-1-yl]-2-isobutoxycarbonylamino-3-oxo-propylsulfanylmethyl}-phosphonic acid
{2-Butoxycarbonylamino-3-[2-(1-carbamoyl-2-naphthalen-2-yl-ethylcarbamoyl)-piperidin-1-yl]-3-oxo-propylsulfanylmethyl}-phosphonic acid
{3-[2-(1-Carbamoyl-2-naphthalen-2-yl-ethylcarbamoyl)-piperidin-1-yl]-2-methoxycarbonylamino-3-oxo-propylsulfanylmethyl}-phosphonic acid
{3-[2-(1-Carbamoyl-2-naphthalen-2-yl-ethylcarbamoyl)-piperidin-1-yl]-3-oxo-2-phenoxycarbonylamino-propylsulfanylmethyl}-phosphonic acid
{3-[2-(1-Carbamoyl-2-naphthalen-2-yl-ethylcarbamoyl)-piperidin-1-yl]-3-oxo-2-phenethyloxycarbonylamino-propylsulfanylmethyl}-phosphonic acid
{2-Benzenesulfonylamino-3-[2-(1-carbamoyl-2-naphthalen-2-yl-ethylcarbamoyl)-piperidin-1-yl]-3-oxo-propylsulfanylmethyl}-phosphonic acid
[3-[2-(1-Carbamoyl-2-naphthalen-2-yl-ethylcarbamoyl)-piperidin-1-yl]-3-oxo-2-(2-phenyl-ethanesulfonylamino)-propylsulfanylmethyl]-phosphonic acid
[3-[2-(1-Carbamoyl-2-naphthalen-2-yl-ethylcarbamoyl)-piperidin-1-yl]-3-oxo-2-(3-phenyl-propane-1-sulfonylamino)-propylsulfanylmethyl]-phosphonic acid
{3-[2-(1-Carbamoyl-2-naphthalen-2-yl-ethylcarbamoyl)-piperidin-1-yl]-2-methanesulfonylamino-3-oxo-propylsulfanylmethyl}-phosphonic acid
[3-[2-(1-Carbamoyl-2-naphthalen-2-yl-ethylcarbamoyl)-piperidin-1-yl]-3-oxo-2-(2.4.6-trimethyl-benzenesulfonylamino)-propylsulfanylmethyl]-phosphonic acid
[3-[2-(1-Carbamoyl-2-naphthalen-2-yl-ethylcarbamoyl)-piperidin-1-yl]-3-oxo-2-(thiophene-2-sulfonylamino)-propylsulfanylmethyl]-phosphonic acid
[3-[2-(1-Carbamoyl-2-naphthalen-2-yl-ethylcarbamoyl)-piperidin-1-yl]-3-oxo-2-(3-piperidin-1-yl-propionylamino)-propylsulfanylmethyl]-phosphonic acid
{2-[2-(1-Carbamoyl-2-naphthalen-2-yl-ethylcarbamoyl)-piperidin-1-yl]-ethylsulfanylmethyl}-phosphonic acid
{2-(2-Benzo[1.3]dioxol-5-yl-acetylamino)-3-[2-(1-carbamoyl-2-naphthalen-2-yl-ethylcarbamoyl)-piperidin-1-yl]-3-oxo-propylsulfanylmethyl}-phosphonic acid
{3-[2-(1-Carbamoyl-2-naphthalen-2-yl-ethylcarbamoyl)-piperidin-1-yl]-2-[2-(3.5-dimethoxy-phenyl)-acetylamino]-3-oxo-propylsulfanylmethyl}-phosphonic acid
{3-[2-(1-Carbamoyl-2-naphthalen-2-yl-ethylcarbamoyl)-piperidin-1-yl]-2-[2-(2-methoxy-phenyl)-acetylamino]-3-oxo-propylsulfanylmethyl}-phosphonic acid
{2-[2-(2-Naphthalen-2-yl-ethylcarbamoyl)-piperidin-1-yl]-2-oxo-ethylsulfanylmethyl}-phosphonic acid
[2-Oxo-2-(2-phenylcarbamoyl-piperidin-1-yl)-ethylsulfanylmethyl]-phosphonic acid
{3-[2-(1-Carbamoyl-2-naphthalen-2-yl-ethylcarbamoyl)-piperidin-1-yl]-2-[2-(3-methoxy-phenyl)-acetylamino]-3-oxo-propylsulfanylmethyl}-phosphonic acid
{3-[2-(1-Carbamoyl-2-naphthalen-2-yl-ethylcarbamoyl)-piperidin-1-yl]-3-oxo-2-[2-(4-piperazin-1-yl-phenyl)-acetylamino]-propylsulfanylmethyl}-phosphonic acid
{2-[2-(1-tert-Butylcarbamoyl-2-naphthalen-2-yl-ethylcarbamoyl)-piperidin-1-yl]-2-oxo-ethylsulfanylmethyl}-phosphonic acid
{2-[2-(1-Methylcarbamoyl-2-naphthalen-2-yl-ethylcarbamoyl)-piperidin-1-yl]-2-oxo-ethylsulfanylmethyl}-phosphonic acid
{2-[2-(1-Carbamoyl-2-naphthalen-2-yl-ethylcarbamoyl)-piperidin-1-yl]-1-methyl-2-oxo-ethylsulfanylmethyl}-phosphonic acid
[2-(2-Benzylcarbamoyl-piperidin-1-yl)-2-oxo-ethylsulfanylmethyl]-phosphonic acid
[2-Oxo-2-(2-phenethylcarbamoyl-piperidin-1-yl)-ethylsulfanylmethyl]-phosphonic acid
{2-Oxo-2-[2-(3-phenyl-propylcarbamoyl)-piperidin-1-yl]-ethylsulfanylmethyl}-phosphonic acid
{2-[2-(1-Carbamoyl-2-naphthalen-2-yl-ethylcarbamoyl)-piperidin-1-yl]-1-methylcarbamoylmethyl-2-oxo-ethylsulfanylmethyl}-phosphonic acid
{2-[2-(1-Carbamoyl-2-naphthalen-2-yl-ethylcarbamoyl)-piperidin-1-yl]-1-[(4-methoxy-phenylcarbamoyl)-methyl]-2-oxo-ethylsulfanylmethyl}-phosphonic acid
{2-[2-(1-tert-Butylcarbamoyl-2-naphthalen-2-yl-ethylcarbamoyl)-piperidin-1-yl]-ethylsulfanylmethyl}-phosphonic acid
2,2-Dimethyl-propionic acid {2-[2-(1-tert-butylcarbamoyl-2-naphthalen-2-yl-ethylcarbamoyl)-piperidin-1-yl]-2-oxo-ethylsulfanylmethyl}-(2,2-dimethyl-propionyloxymethoxy)-phosphinoyloxymethyl ester
{2-[2-(2-Naphthalen-2-yl-1-phenyl-ethylcarbamoyl)-piperidin-1-yl]-2-oxo-ethylsulfanylmethyl}-phosphonic acid
(2-{2-[1-(4-Methyl-thiazol-2-yl)-2-naphthalen-2-yl-ethylcarbamoyl]-piperidin-1-yl}-2-oxo-ethylsulfanylmethyl)-phosphonic acid
{2-[2-(2-Naphthalen-2-yl-1-[1,3,4]oxadiazol-2-yl-ethylcarbamoyl)-piperidin-1-yl]-2-oxo-ethylsulfanylmethyl}-phosphonic acid
(2-{2-[2-Naphthalen-2-yl-1-(4H-[1,2,4]triazol-3-yl)-ethylcarbamoyl]-piperidin-1-yl}-2-oxo-ethylsulfanylmethyl)-phosphonic acid
{2-[2-(2-Naphthalen-2-yl-1-pyridin-2-yl-ethylcarbamoyl)-piperidin-1-yl]-2-oxo-ethylsulfanylmethyl}-phosphonic acid
and salts, hydrates and solvates thereof as well as pro drugs thereof.

14. A pharmaceutical composition comprising a compound according to claim 1 and a pharmaceutically acceptable carrier, diluent or excipient.

15. The pharmaceutical composition according to claim 14 comprising a further pharmaceutically active compound.

16. The pharmaceutical composition according to claim 14, wherein the compound is present as a pharmaceutically acceptable salt or a pharmaceutically active solvate.

17. The pharmaceutically active composition according to claim 14, wherein the pharmaceutically active compound is either alone or in combination with any of the ingredients of the composition present in a multitude of individualized dosages and/or administration forms.

18. Use of a compound according to claim 1 for the manufacture of a medicament.

19. Use of a compound for the manufacture of a medicament for the treatment of a disease, whereby the disease involves an abnormal cell proliferation, an undesired cell proliferation, an abnormal mitosis and/or an undesired mitosis,
whereby the compound is a compound according to claim 1.

20. The use according to claim 19, wherein the compound is acting on an enzymatic activity involved in the regulation of cell division and/or cell cycle or part thereof, preferably the part of the cell cycle is mitosis.

21. The use according to claim 19, wherein the disease is selected from the group comprising neurodegenerative diseases, stroke, inflammatory diseases, immune based disorders, infectious diseases, heart diseases, cardiovascular diseases and cell proliferative diseases.

22. The use according to claim 21, wherein the neurodegenerative disease is selected from the group comprising Alzheimer's disease, Huntington's disease, Parkinson's disease, peripheral neuropathy, progressive supranuclear palsy, corticobasal degeneration, frontotemporal dementia, synucleinopathies, multiple system atrophy, amyotrophic lateral atrophy, prion diseases and motor neuron diseases.

23. The use according to claim 21, wherein the infectious disease is selected from the group comprising fungal, viral, bacterial and parasite infection.

24. The use according to claim 23, wherein the fungal infection is selected from the group comprising gynaecological and dermatological infection.

25. The use according to claim 23, wherein the fungal infection is caused by or involves Histoplasma, Coccidioides, Cryptococcus, Blastomyces, Paracoccidioides, Aspergillus, Sporothrix, Rhizopus, Absidia, Mucor, Hormodendrum, Phialophora Microsporum, Epidermophyton, Rhinosporidum or by a yeast, preferably Candida or Cryptococcus.

26. The use according to claim 21, wherein the infectious disease is selected from or the fungal infection causes a disorder selected from the group comprising ringworm, candidiasis, coccidioidomycosis, blastomycosis, aspergillosis, cryptococcosis, histioplasmosis, paracoccidiomycosis, zygomycosis, sporotrichiosis, mycotic keratitis, nail hair and skin disease, lobomycosis, chromoblastomycosis, mycetoma.

27. The use according to claim 23, wherein the bacterial infection is selected from the group comprising infections caused by Gram-positive and by Gram-negative bacteria.

28. The use according to claim 27, wherein the bacterial infection is caused by or involves Staphylococcus, Clostridium, Streptococcus, Listeria, Salmonella, Bacillus, Escherichia, Mycobacteria, Serratia, Enterobacter, Enterococcus, Nocardia, Hemophilus, Neisseria, Proteus, Yersinia, Helicobacter or Legionella.

29. The use according to claim 21, wherein the infectious disease is selected from or the bacterial infection causes a disorder selected from the group comprising pneumonia, diarrhea, dysentery, anthrax, rheumatic fever, toxic shock syndrome, mastoiditis, meningitis, gonorrhea, typhoid fever, brucellis, Lyme disease, gastroenteritis, tuberculosis, cholera, tetanus and bubonic plague.

30. The use according to claim 23, wherein the viral infection is selected from the group comprising infections caused by or involving retrovirus, HIV, Papilloma virus, Polio virus, Epstein-Barr, Herpes virus, Hepatitis virus, Papova virus, Influenza virus, Rabies, JC, encephalitis causing virus or hemorrhagic fever causing virus.

31. The use according to claim 23, wherein the parasite infection is selected from the group comprising infections caused by or involving Trypanosoma, Leishmania, Trichinella, Echinococcus, Nematodes, Classes Cestoda Trematoda, Monogenea, Toxoplasma, Giardia, Balantidium, Paramecium, Plasmodium, or Entamoeba.

32. The use according to claim 21, wherein the cell proliferative disorder is selected from the group comprising neoplastic and non-neoplastic disorders.

33. The use according to claim 32, wherein the neoplastic cell proliferative disorder is selected from the group comprising solid tumor, lymphoma and leukemia.

34. The use according to claim 33, wherein the solid tumor is selected from the group comprising carcinoma, sarcoma, osteoma, fibrosarcoma, and chondrosarcoma.

35. The use according to claim 32, wherein the neoplastic cell proliferative disorder is selected from the group comprising breast cancer, prostate cancer, colon cancer, brain cancer, lung cancer, pancreatic cancer, gastric cancer, bladder cancer and kidney cancer.

36. The use according to claim 32, wherein the non-neoplastic cell proliferative disorder is a fibrotic disorder, preferably the fibrotic disorder is fibrosis.

37. The use according to claim 32, wherein the non-neoplastic cell proliferative disorder is selected from the group comprising prostatic hypertrophy, endometriosis, psoriasis, tissue repair and wound healing.

38. The use according to claim 21, wherein the immune based/inflammatory disease is an autoimmune disease or disorder.

39. The use according to claim 21, wherein the immune based/inflammatory disease is selected from the group comprising rheumatoid arthritis, glomerulonephritis, systemic lupus erythematosus associated glomerulonephritis, irritable bowel syndrome, bronchial asthma, multiple sclerosis, pemphigus, pemphigoid, scleroderma, myasthenia gravis, autoimmune haemolytic and thrombocytopenic states, Goodpasture's syndrome, pulmonary hemorrhage, vasculitis, Crohn's disease and dermatomyositis.

40. The use according to claim 21, wherein the immune based and/or inflammatory disease is an inflammatory condition.

41. The use according to claim 21, wherein the immune based and/or inflammatory disease is selected from the group comprising inflammation associated with burns, lung injury, myocardial infarction, coronary thrombosis, vascular occlusion, post-surgical vascular reocclusion, artherosclerosis, traumatic central nervous system injury, ischemic heart disease and ischemia-reperfusion injury, acute respiratory distress syndrome, systemic inflammatory response syndrome, multiple organ dysfunction syndrome, tissue graft rejection and hyperacute rejection of transplanted organs.

42. The use according to claim 19, wherein the medicament is for administration via an administration route which is selected from the group comprising oral, subcutaneous, intravenous, intranasal, transdermal, intraperitoneal, intramuscular, intrapulmonar, vaginal, rectal, and intraocular administration.

43. The use according to claim 19, wherein the medicament is for the administration to a mammal, preferably to a human being.

44. (canceled)


SEQUENCE LISTING (2 sequences)

SEQ ID NO: 1 - protein, 5 aa, Artificial Sequence
  substrate for hPin1 PPIase
AASPR

SEQ ID NO: 2 - protein, 4 aa, Artificial Sequence
  substrate for PPIase
AFPF